(19) United States
(12) Patent Application Publication
(10) Pub. No.: US 2024/0398891 A1
(43) Pub. Date: Dec. 5, 2024

(54) COMPOSITIONS AND METHODS OF TREATING MELANOMA

(71) Applicant: UNIVERSITY OF IOWA RESEARCH FOUNDATION, Iowa City, IA (US)

(72) Inventors: Michael K. Schultz, Iowa City, IA (US); Frances L. Johnson, Iowa City, IA (US); Somya Kapoor, Iowa City, IA (US); Dongyoul Lee, Iowa City, IA (US); Mengshi Li, Iowa City, IA (US); Molly Martin, Iowa City, IA (US)

(73) Assignee: UNIVERSITY OF IOWA RESEARCH FOUNDATION, Iowa City, IA (US)

(21) Appl. No.: 18/750,667

(22) Filed: Jun. 21, 2024

Related U.S. Application Data
(63) Continuation of application No. 17/531,485, filed on Nov. 19, 2021.
Application No. 17/531,485 is a continuation-in-part of application No. 16/312,846, filed on Dec. 21, 2018, now Pat. No. 11,179,484.
(60) Provisional application No. 62/354,345, filed on Jun. 24, 2016.
(60) Provisional application No. 62/370,125, filed on Aug. 2, 2016.

(51) Int. Cl. A61K 38/12; A61K 47/54; A61K 47/60; A61K 51/04; A61P 35/00; C07K 16/28

(52) U.S. Cl. CPC A61K 38/12; A61K 47/547; A61K 47/60; A61K 51/0482; A61P 35/00; C07K 16/2818; C07K 16/2827

(57) ABSTRACT

The invention provides compositions, kits and methods to treat a hyperproliferative disorder with an agent that increases expression of MCR1 and an MCR1 ligand. The invention also provides a method of treating drug-resistant melanoma, comprising administering an MCR1 ligand to a patient in need thereof.

The present invention also provides in certain embodiments a melanoma-targeting conjugate comprising Formula I:

T-L-X

wherein T is a MCR1 ligand, L is a linker, and X an anti-cancer composition, for the therapeutic treatment of a hyperproliferative disorder. The present invention also provides methods, kits, and uses of the conjugate of Formula I.

### RELATED APPLICATIONS

This application claims priority to U.S. patent application Ser. No. 17/531,485, filed Nov. 19, 2021, which claims priority to U.S. patent application Ser. No. 16/312,846, filed Dec. 21, 2018, which is a 35 U.S.C. § 371 application of International Application Serial No. PCT/US2017/039299, filed Jun. 26, 2017, which claims priority to U.S. Provisional Application No. 62/354,345 that was filed on Jun. 24, 2016, and U.S. Provisional Application No. 62/370,125 that was filed on Aug. 2, 2016. The entire content of the applications referenced above are hereby incorporated by reference.

### FEDERAL GRANT SUPPORT

The invention was made with government support under K25CA172218-01A1 awarded by the National Institutes of Health. The government has certain rights in the invention.

### BACKGROUND

Melanoma is a cancer of the skin and is the fastest growing cancer incidence in the world today. Disease detected early can be removed by surgery, but when melanoma spreads to other parts of the body (called metastatic melanoma) it is almost uniformly fatal. The reason for this is that metastatic melanoma rapidly becomes resistance to all forms of treatment. One of the first new pharmaceutical therapies that appeared effective for melanoma (called vemurafenib) was approved in 2011. Vemurafenib targets patients with a gene mutation (BRAF^{V600E}) that is present in about half of melanoma patients. Although these patients respond well to the treatment, melanoma develops resistance to the therapy rapidly. Thus, the new therapy, which initially was heralded as the end of melanoma, extends life expectancy by only months. Vemurafenib is one of several BRAF inhibitors that are being used for melanoma therapy that target the BRAF protein. These BRAF inhibitors are now often used in combination with other inhibitors of proteins in the mitogen-activated protein kinase MAPK) pathway, a signaling pathway that is implicated in the cancerous phenotype of melanoma and other cancers. The MAPK pathway plays a role in the regulation of gene expression, cellular growth, and survival. Abnormal MAPK signaling may lead to increased or uncontrolled cell proliferation and resistance to apoptosis. Melanoma develops resistance to all of these therapies.

Recent introductions of a second class of drugs has resulted in approvals of new immunotherapies targeting regulator proteins of the immune system, which includes the recent development of anti-CTLA-4 monoclonal antibodies, Toll-like receptor (TLR) agonists, CD40 agonists, and anti-ganglioside monoclonal antibodies. These include CTLA-4 and PD1 inhibitors. Several other drugs that have different mechanisms of action are also approved for melanoma treatment, but the disease eventually develops resistance to all therapies for melanoma. There is no treatment for metastatic melanoma that overcomes resistance of melanoma cancer cells, which leads to a high mortality rate and the 5 year survival for patients diagnosed with metastatic melanoma is less than 20%.

Radiopharmaceuticals are radiolabeled drugs that are used for imaging and therapy of disease. These drugs are designed of the form C-L-T, where C is a chelator that stably complexes (i.e., binds tightly) a radionuclide (e.g., Pb-212, Ac-225, Lu-177, Cu-64, Cu-67, Ga-68, Pb-203) that decays by various forms of radioactive decay modes (e.g., beta-particle emission, alpha-particle emission, positron emission, gamma-ray emission, auger electron emission); T is a targeting molecular structure (e.g., peptide, antibody, small molecule, aptamer) that is designed to bind to diseased cells, often by binding to a cell surface receptor (e.g., g-protein coupled receptor, type II glycoprotein, or other antigen); and L is a molecular linker that connects the chelator C to the binding moiety T.

The use of certain radionuclides that emit gamma rays enables imaging that is used often for diagnosing and monitoring of disease. Other radionuclides that emit particles, such as beta and alpha particles, are used for treating diseases, such as cancers. In some cases, the radionuclides intended for the treatment of cancer or other diseases, decay further to a series of radionuclide progeny (often referred to as daughter radionuclides or “daughters”) that may or may not be complexed by the chelator. The preparation of radiopharmaceuticals involves a reaction of the C-L-T precursor with the radionuclide. Examples of radionuclides used for this purpose that have a series of daughter radionuclide progeny in their series includes Pb-212 and Ac-225.

There is a need in the industry for an effective means of treating cancer. The present inventors have discovered a means of treating cancer through the use of unique chelating compounds.

### SUMMARY

The present invention describes provides in certain embodiments a melanoma-targeting conjugate comprising Formula I:

T-L-X
wherein T is a radiolabeled MCR1 ligand,
L is a linker, and
X an anti-cancer composition,
for the therapeutic treatment of melanoma.

In certain embodiments, the radiolabeled MCR1 ligand is a peptide, or antibody or antibody fragment, or a small molecule.

In certain embodiments, T is Re[Cys-Cys-Glu-His-D-Phe-Arg-Trp-Cys-Arg-Pro-Val-NH₂].

In certain embodiments, the MCR1 ligand is radiolabeled with a radionuclide that is used for medical imaging and/or therapy of the cancerous tumors.

In certain embodiments, the radionuclide is Ga-68; In-111; Pb-203; F-18; C-11; Zr-89; Sc-44; Tc-99m or other medical radionuclide used for imaging.

In certain embodiments, the radionuclide is Y-90; Pb-212; Bi-212; Bi-213; At-211; Lu-177; Re-188; or other medical radionuclide used to treat the cancerous tumors.

In certain embodiments, L is a chemical linker that is inserted into a position between the peptide backbone that recognizes the MCR1 protein and the chelator that is used to radiolabel the composition using radionuclides for diagnostic imaging and/or therapy; and the linker improves the internalization of the composition into cells and improves the retention of the composition in tumors for more precise delivery of radiation to the cancerous tissue.

In certain embodiments, L is a hydrophobic linker consisting of an aliphatic carbon chain that connects the chelator to the peptide backbone.

In certain embodiments, L is a hydrophilic linker that includes heteroatom substitutions in the aliphatic chain that connects the chelator to the peptide backbone.

In certain embodiments, L is a mixture of hydrophilic and hydrophobic entities including piperidine insertions of amino acid insertions to lengthen the chain and modulate the pharmacodynamics properties of the composition.

In certain embodiments, L is PEGₙ, wherein n is 1-10. In certain embodiments, n is 2, 4 or 8 PEG subunits. In certain embodiments, n is 4. (FIG. 9)

In certain embodiments, L is an aliphatic (ALP) linker of 2 or 4 carbons. (FIG. 9)

In certain embodiments, L is a piperidine (PIP) based linker with mixed characteristics. (FIG. 9)

In certain embodiments, X is a chelating agent (also called a “chelator”).

In certain embodiments, X is radiolabeled with a radionuclide that is used for medical imaging and/or therapy of the cancerous tumors.

In certain embodiments, the chelator is radiometallated or radiolabeled with a radionuclide that is suitable for the therapeutic treatment and radiologic (or non-radiologic) imaging of melanoma or other MCR1 expression cancerous malignancy (e.g., medulloblastoma).

In certain embodiments, the radionuclide is Ga-68; In-111; Pb-203; F-18; C-11; Zr-89; Sc-44; Tc-99m or other medical radionuclide used for imaging.

In certain embodiments, the radionuclide is Y-90; Pb-212; Bi-212; Bi-213; At-211; Lu-177; Re-188; or other medical radionuclide used to treat the cancerous tumors.

In certain embodiments, the chelating agent is DOTA or other chelator that is used to bind the radionuclide for diagnostic imaging or therapy for cancer or other disease.

In certain embodiments, the chelator is based on S-2-(4-Nitrobenzyl)-1,4,7,10-tetraazacyclododecane or other variation on this cyclododecane.

In certain embodiments, the chelator is based on 1,4,7,10-Tetraazacyclododecane-1,4,7-tri(carbamoylmethyl)-10-acetic acid.

In certain embodiments, the chelator is based on S-2-(4-Nitrobenzyl)-1,4,7,10-tetraazacyclododecane tetraacetic acid.

In certain embodiments, the chelator is based on S-2-(4-Aminobenzyl)-1,4,7,10-tetraazacyclododecane tetraacetic acid.

In certain embodiments, the chelator is based on S-2-(4-Aminobenzyl)-1,4,7,10-tetraazacyclododecane tetra-tert-butylacetate.

In certain embodiments, the chelator is based on S-2-(4-Isothiocyanatobenzyl)-1,4,7,10-tetraazacyclododecane tetraacetic acid.

In certain embodiments, the chelator is based on 1,4,7,10-Tetraazacyclododecane-1,4,7-tris-tert-butyl acetate-10-acetic acid.

In certain embodiments, the chelator is based on 1,4,7,10-Tetraazacyclododecane-1,4,7-tris-tert-butyl acetate-10-succinimidyl acetate.

In certain embodiments, the chelator is based on 1,4,7,10-Tetraazacyclododecane-1,4,7-tris-tert-butyl acetate-10-maleimidoethylacetamide.

In certain embodiments, the chelator is based on 1,4,7,10-Tetraazacyclododecane-1,4,7-tris-acetic acid-10-maleimidoethylacetamide.

In certain embodiments, the chelator is based on 1,4,7,10-Tetraazacyclododecane-1,4,7-tris-tert-butyl acetate-10-(N-a-Fmoc-N-e-acetamido-L-lysine).

In certain embodiments, the chelator is based on 1,4,7,10-Tetraazacyclododecane-1,4,7-tris(t-butyl acetate)-10-(3-butynylacetamide).

In certain embodiments, the chelator is based on 1,4,7,10-Tetraazacyclododecane-1,4,7-tris(t-butyl-acetate)-10-(amino ethylacetamide).

In certain embodiments, the chelator is based on 1,4,7,10-Tetraazacyclododecane-1,4,7-tris-tert-butyl acetate-10-(azidopropyl ethylacetamide).

In certain embodiments, the chelator is based on 1,4,7,10-Tetraazacyclododecane-1,4,7-tris(t-butyl acetate)-10-(4-aminobutyl)acetamide.

In certain embodiments, the chelator is based on 1,4,7,10-Tetraazacyclododecane-1,4,7,10-tetraacetic acid mono-N-hydroxysuccinimide ester.

In certain embodiments, the chelator is based on 1,4,7,10-Tetraazacyclododecane-1,4,7-tris(acetic acid)-10-(2-thioethyl)acetamide or other variation of DOTA.

In certain embodiments, the chelator is based on S-2-(4-Aminobenzyl)-diethylenetriamine pentaacetic acid or other variation of DTPA.

In certain embodiments, the chelator is based on 3,6,9,15-Tetraazabicyclo[9.3.1]pentadeca-1(15),11,13-triene-4-S-(4-aminobenzyl)-3,6,9-triacetic acid or other variation on this pentadeca macrocycle.

In certain embodiments, the chelator is based on 1-Oxa-4,7,10-tetraazacyclododecane-5-S-(4-aminobenzyl)-4,7,10-triacetic acid or other variation on oxo-substituted macrocycle.

In certain embodiments, the chelator is based on 2-S-(4-Isothiocyanatobenzyl)-1,4,7-triazacyclononane-1,4,7-triacetic acid or other variation on this cyclononane.

In certain embodiments, the chelator is based on 1-(4-isothiocyanatophenyl)-3-[6,17-dihydroxy-7,10,18,21-tetraoxo-27-(N-acetylhydroxylamino)-6,11,17, 22-tetraazaheptaeicosine] thiourea or other variation on deferoxamine.

The present invention provides in certain embodiments a conjugate consisting of DOTA-PEG4-VMT-(MCR1 ligand).

In certain embodiments, the present invention consists of DOTA-PEG4-Re[Cys-Cys-Glu-His-D-Phe-Arg-Trp-Cys-Arg-Pro-Val-NH2].

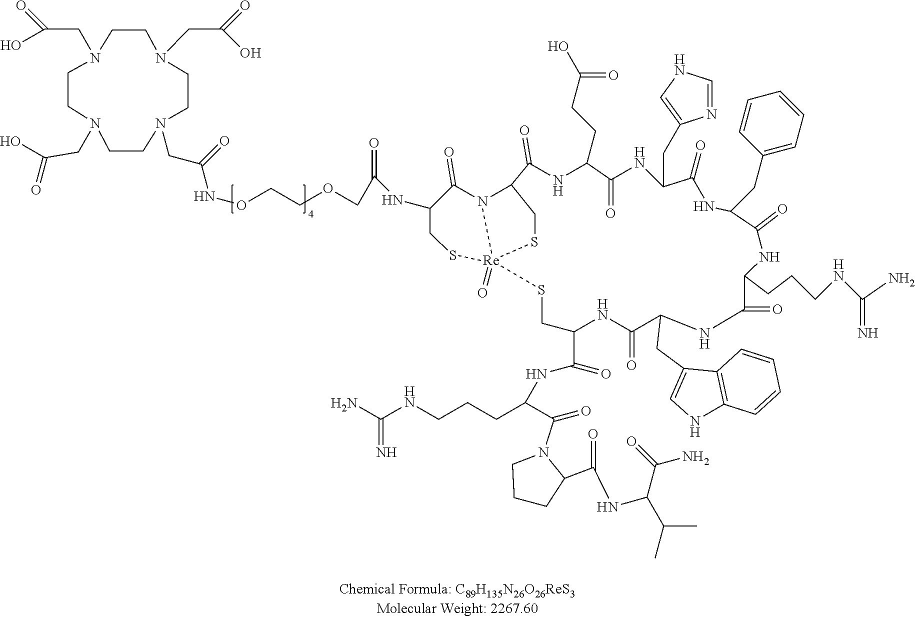

In certain embodiments, the present invention consists of the conjugate VMT1 (FIG. 22A), VMT2 (FIG. 22B), or PSC-PEG-CLICK (FIG. 22C).

In certain embodiments, DOTA is radiolabeled.

In certain embodiments, the present invention consists of bombesin conjugates VMT-BBN-001 (FIG. 23A), VMT-BBN-002 (FIG. 23B), VMT-BBN-003 (FIG. 23C), VMT-BBN-004 (FIG. 23D), VMT-BBN-005 (FIG. 23E), and PSC-RM2 (FIG. 23F).

In certain embodiments, the radiolabel is Pb-203.

The present invention provides in certain embodiments a method of treating hyperproliferative disorder in a patient in need thereof, comprising administering the conjugate described above. In certain embodiments, the hyperproliferative disorder is melanoma. In certain embodiments, the hyperproliferative disorder is a cancer characterized by increased In certain embodiments, the conjugate is administered orally or parenterally.

In certain embodiments, the method further comprises administering an anti-cancer composition.

In certain embodiments, the anti-cancer composition comprises phenyl butyric acid (PBA) or a pharmaceutically acceptable salt thereof, chloroquine, hydroxychloroquine (laquenil, Axemal (in India), Dolquine and Quensyl, or a pharmaceutical drug that is an antimalarial or inhibits interactions between lysosomes and autophagasomes that overcome resistance that is linked to autophagy; derivative of triphenylphosphonium (TPP), PBA, a histone deacetylation inhibitor, a MAPK pathway inhibitor, such as a MEK inhibitor, a RAS inhibitor, and/or RAF inhibitor.

In certain embodiments, the present invention further comprises administering an agent that increases expression of MCR1.

In certain embodiments, the present invention further comprises administering an immunotherapy targeting regulator protein of the immune system. In certain embodiments, the immunotherapy includes an anti-CTLA-4 monoclonal antibody, Toll-like receptor (TLR) agonist, CD40 agonist, and/or anti-ganglioside monoclonal antibody. In certain embodiments, the immunotherapy includes CTLA-4 and PD1 inhibitors.

In certain embodiments, the hyperproliferative disorder is melanoma.

In certain embodiments, the agent that increases expression of MCR1 is vemurafenib, PBA, a histone deacetylation inhibitor and/or another MAPK pathway inhibitor, a RAS inhibitor, and/or RAF inhibitor.

In certain embodiments, the histone deacetylase inhibitor is Vorinastat.

In certain embodiments, the MAPK pathway inhibitor is a MEK inhibitor.

In certain embodiments, the MEK inhibitor is cobimetinib or trametinib.

In certain embodiments, the agent that increases expression of MCR1 is administered separately, sequentially or simultaneously with the conjugate.

In certain embodiments, the agent that increases expression of MCR1 is administered from about one to about six month before the administration of the conjugate.

In certain embodiments, the agent is administered orally or parenterally.

In certain embodiments, the agent is administered subcutaneously.

In certain embodiments, the conjugate is administered orally or parenterally.

In certain embodiments, administration of the agent begins about 1 to about 10 days before administration of the conjugate.

In certain embodiments, administration of the agent and administration of the conjugate begin on the same day.

In certain embodiments, the method further comprises administering an anti-cancer composition.

In certain embodiments, the anti-cancer composition comprises phenyl butyric acid (PBA) or a pharmaceutically acceptable salt thereof, chloroquine, hydroxychloroquine (laquenil, Axemal (in India), Dolquine and Quensyl, or a pharmaceutical drug that is an antimalarial or inhibits interactions between lysosomes and autophagasomes that overcome resistance that is linked to autophagy; derivative of triphenylphosphonium (TPP), PBA, a histone deacetylation inhibitor, a MAPK pathway inhibitor, such as a MEK inhibitor, a RAS inhibitor, and/or RAF inhibitor.

In certain embodiments, the histone deacetylation inhibitor is Vorinastat.

In certain embodiments, the MAPK pathway inhibitor is a MEK inhibitor.

In certain embodiments, the MEK inhibitor is cobimetinib or trametinib.

In certain embodiments, the conjugate is administered in a single dose.

In certain embodiments, the conjugate is administered in multiple doses.

In certain embodiments, the conjugate is administered sequentially daily for several days.

In certain embodiments, the conjugate is administered once per week for 1 month.

In certain embodiments, the conjugate is administered once per week for up to 6 months.

In certain embodiments, the conjugate is administered in a dose of 1 mCi for medical imaging.

In certain embodiments, the conjugate is administered in a dose of up to 10 mCi for medical imaging.

In certain embodiments, the conjugate is administered in a dose of up to 50 mCi for medical imaging.

In certain embodiments, the conjugate is administered in a dose of 0.1 mCi for medical treatment of the cancerous tumors.

In certain embodiments, the conjugate is administered in a dose of up to 1 mCi for medical treatment of the cancerous tumors.

In certain embodiments, the conjugate is administered in a dose of up to 10 mCi for medical treatment of the cancerous tumors.

In certain embodiments, the conjugate is administered in a dose of up to 100 mCi for medical treatment of the cancerous tumors.

In certain embodiments, the conjugate is administered for more than a month.

In certain embodiments, the conjugate is administered for more than a year.

In certain embodiments, the conjugate is administered at a dosage of at least 1500 mg/day.

The present invention provides in certain embodiments a kit comprising the conjugate described above, a container, and a package insert or label indicating the administration of the conjugate with vemurafenib for treating melanoma.

The present invention provides in certain embodiments a product comprising the conjugate described above, and vemurafenib; as a combined preparation for separate, simultaneous or sequential use in the treatment of melanoma.

The present invention provides in certain embodiments a method of treating drug-resistant melanoma, comprising administering the conjugate described above to a patient in need thereof.

In certain embodiments, the melanoma is resistant to vemurafenib treatment.

The present invention provides in certain embodiments a use of the conjugate described above; and one or more anti-cancer agents for the therapeutic treatment of melanoma.

In certain embodiments, the cancer is vemurafenib-resistant melanoma.

The present invention provides in certain embodiments a use of the conjugate described above wherein:
a) the conjugate is administered simultaneously with the one or more anti-cancer agents; or
b) the conjugate and the one or more anti-cancer agents are administered sequentially; or
c) administration of the one or more anti-cancer agents begins about 1 to about 10 days before administration of the conjugate; or
d) administration of the conjugate thereof begins about 1 to about 10 days before administration of the one or more anti-cancer agents; or
e) administration of conjugate and administration of the one or more anti-cancer agents begins on the same day.

In certain embodiments, the conjugate is administered in combination with vemurafenib, and the cancer is melanoma.

In certain embodiments, conjugate, is administered in combination with vemurafenib and chloroquine, and the cancer is melanoma.

The present invention provides in certain embodiments, a method of treating a cell that has upregulated MCR1 expression as compared to a comparable wildtype cell comprising contacting the cell with an MCR1 ligand or with the conjugate described above. As used herein, an “MCR1 ligand” is a ligand that binds specifically to the MCR1 receptor.

In certain embodiments, the upregulation is a result of prior contact with vemurafenib, PBA, a histone deacetylation inhibitor and/or another MEK inhibitor.

In certain embodiments, the upregulation is a result of prior contact with vemurafenib.

In certain embodiments, the upregulation is a result of prior contact with PBA.

In certain embodiments, the ligand is a peptide.

In certain embodiments, the peptide is radiolabeled.

The present invention provides in certain embodiments, a method of treating hyperproliferative disorder in a patient in need thereof, comprising (a) administering an agent that increases expression of MCR1, and (b) administering an MCR1 ligand.

In certain embodiments, the hyperproliferative disorder is melanoma.

In certain embodiments, the agent that increases expression of MCR1 is vemurafenib, PBA, a histone deacetylation inhibitor, such as Vorinastat or other histone deacetylase inhibitor, and/or another MAPK pathway inhibitor, such as a MEK inhibitor (e.g., cobimetinib, trametinib), a RAS inhibitor, and/or RAF inhibitor.

In certain embodiments, the MCR1 ligand is a peptide.

In certain embodiments, the peptide is radiolabeled.

In certain embodiments, the agent that increases expression of MCR1 is administered separately, sequentially or simultaneously with the MCR1 ligand.

In certain embodiments, the agent that increases expression of MCR1 is administered from about one day to about 6 months before the administration of the MCR1 ligand.

In certain embodiments, the agent is administered orally or parenterally.

In certain embodiments, the agent is administered subcutaneously.

In certain embodiments, the MCR1 ligand is administered orally or parenterally.

In certain embodiments, the administration of the agent begins about 1 to about 10 days before administration of the MCR1 ligand.

In certain embodiments, the administration of the agent and administration of the MCR1 ligand begin on the same day.

In certain embodiments, the method further comprises administering an anti-cancer composition.

In certain embodiments, the anti-cancer composition comprises a combination of phenyl butyric acid or one of its salts such as sodium phenylbutyrate (referred to collectively as PBA) or a pharmaceutically acceptable salt thereof, chloroquine, hydroxychloroquine (laquenil, Axemal (in India), Dolquine and Quensyl, or a pharmaceutical drug that is an antimalarial or inhibits interactions between lysosomes and autophagasomes that overcome resistance that is linked to autophagy; and MAPK pathway inhibitors such as vemurafenib, cobimetinib, and/or other inhibitors of the MAPK pathway, a derivative of triphenylphosphonium (TPP), PBA, a histone deacetylation inhibitor, such as Vorinastat or other histone deacetylase inhibitor, and/or another MAPK pathway inhibitor, such as a MEK inhibitor (e.g., cobimetinib, trametinib), a RAS inhibitor, and/or RAF inhibitor.

In certain embodiments, the combination includes a radiolabeled MCR1 ligand that is designed to bind to the MCR1 protein on or in cells in the cancerous tumors of the patient.

In certain embodiments, the MCR1 ligand is radiolabeled with a radionuclide that is used for medical imaging and/or therapy of the cancerous tumors by techniques such as single photon emission computed tomography (SPECT) or positron emission computed tomography (PET).

In certain embodiments, the radionuclide is Ga-68; In-11; Pb-203; F-18; C-11; Zr-89; Sc-44; Tc-99m or other medical radionuclide used for imaging.

In certain embodiments, the radionuclide is Y-90; Pb-212; Bi-212; Bi-213; At-211; Lu-177; Re-188; or other medical radionuclide used to treat the cancerous tumors.

In certain embodiments, the radiolabeled MCR1 ligand is administered in a single dose.

In certain embodiments, the radiolabeled MCR1 ligand is administered in multiple doses.

In certain embodiments, the radiolabeled MCR1 ligand is administered sequentially daily for several days.

In certain embodiments, the radiolabeled MCR1 ligand is administered once per week for 1 month.

In certain embodiments, the radiolabeled MCR1 is administered once per week for up to 6 months.

In certain embodiments, the radiolabeled MCR1 ligand is administered in a dose of 1 mCi for medical imaging.

In certain embodiments, the radiolabeled MCR1 ligand is administered in a dose of up to 10 mCi for medical imaging.

In certain embodiments, the radiolabeled MCR1 ligand is administered in a dose of up to 50 mCi for medical imaging.

In certain embodiments, the radiolabeled MCR1 ligand is administered in a dose of 0.1 mCi for medical treatment of the cancerous tumors.

In certain embodiments, the radiolabeled MCR1 ligand is administered in a dose of up to 1 mCi for medical treatment of the cancerous tumors.

In certain embodiments, the radiolabeled MCR1 ligand is administered in a dose of up to 10 mCi for medical treatment of the cancerous tumors.

In certain embodiments, the radiolabeled MCR1 ligand is administered in a dose of up to 100 mCi for medical treatment of the cancerous tumors.

The present invention provides in certain embodiments, a method of treating drug-resistant melanoma, comprising administering an MCR1 ligand to a patient in need thereof.

In certain embodiments, the melanoma is resistant to vemurafenib treatment.

The present invention provides in certain embodiments, a combination of a) an agent that increases expression of MCR1, and b) MCR1 ligand for the prophylactic or therapeutic treatment of hyperproliferative disorder.

In certain embodiments, the hyperproliferative disorder is melanoma.

In certain embodiments, the combination provides a synergistic effect in treating the hyperproliferative disorder.

In certain embodiments, the agent that increases expression of MCR1 is vemurafenib, PBA, a histone deacetylation inhibitor and/or another MEK inhibitor.

In certain embodiments, the agent that increases expression of MCR1 is vemurafenib.

In certain embodiments, the agent that increases expression of MCR1 is PBA.

In certain embodiments, the MCR1 ligand is a peptide.

In certain embodiments, the peptide is radiolabeled.

The present invention provides in certain embodiments, a method of treating melanoma in a patient in need thereof that has received treatment with vemurafenib, PBA, a histone deacetylation inhibitor and/or another MEK inhibitor, or other MAPK pathway inhibitor, comprising administering an agent that increases expression of MCR1 in combination with an MCR1 ligand to the patient.

The present invention provides in certain embodiments, a kit comprising an agent that increases expression of MCR1, a MCR1 ligand, a container, and a package insert or label indicating the administration of the agent with the MCR1 ligand for treating a hyperproliferative disorder.

As a combined treatment the combination treatment effectively destroys metastatic melanoma cancer cells. In certain embodiments, the hyperproliferative disorder is cancer. In certain embodiments, the cancer is drug-resistant. As used herein, the term “drug-resistant” is reduction in effectiveness of a drug in killing malignant cells; reducing cancerous tumor size and rate of growth; and ameliorating the symptoms a disease or condition. In certain embodiments, the drug's effectiveness is reduced by at least about 10%, 20%, 30%, 40%, 50%, 60%, 70%, 80%, 90%, or even 100%, as compared to its effects when first administered to the mammal.

In certain embodiments, the cancer is melanoma. In certain embodiments, the melanoma is resistant to vemurafenib treatment.

In certain embodiments, the present invention provides a method of treating a cell that has upregulated MCR1 expression as compared to a comparable wildtype cell comprising contacting the cell with an MCR1 ligand or the conjugate of described above.

In certain embodiments, the upregulation is a result of prior contact with vemurafenib, PBA, a histone deacetylation inhibitor and/or another MEK inhibitor.

In certain embodiments, the upregulation is a result of prior contact with vemurafenib.

In certain embodiments, the upregulation is a result of prior contact with PBA.

In certain embodiments, the present invention provides a method of treating hyperproliferative disorder in a patient in need thereof, comprising (a) administering an agent that increases expression of MCR1, and (b) administering the conjugate as described above.

In certain embodiments, the hyperproliferative disorder is melanoma.

In certain embodiments, the agent that increases expression of MCR1 is vemurafenib, PBA, a histone deacetylation inhibitor and/or another MAPK pathway inhibitor, a RAS inhibitor, and/or RAF inhibitor.

In certain embodiments, the histone deacetylase inhibitor is Vorinastat.

In certain embodiments, the MAPK pathway inhibitor is a MEK inhibitor.

In certain embodiments, the MEK inhibitor is cobimetinib or trametinib.

In certain embodiments, the agent that increases expression of MCR1 is administered separately, sequentially or simultaneously with the MCR1 ligand.

In certain embodiments, the agent that increases expression of MCR1 is administered from about one to about six months before the administration of the conjugate.

In certain embodiments, the agent is administered orally or parenterally.

In certain embodiments, the agent is administered subcutaneously.

In certain embodiments, the conjugate is administered orally or parenterally.

In certain embodiments, administration of the agent begins about 1 to about 10 days before administration of the conjugate.

In certain embodiments, administration of the agent and administration of the conjugate begin on the same day.

In certain embodiments, the method further comprises administering an anti-cancer composition.

In certain embodiments, the anti-cancer composition comprises phenyl butyric acid (PBA) or a pharmaceutically acceptable salt thereof, chloroquine, hydroxychloroquine (laquenil, Axemal (in India), Dolquine and Quensyl, or a pharmaceutical drug that is an antimalarial or inhibits interactions between lysosomes and autophagasomes that overcome resistance that is linked to autophagy; derivative of triphenylphosphonium (TPP), PBA, a histone deacetylation inhibitor, a MAPK pathway inhibitor, such as a MEK inhibitor, a RAS inhibitor, and/or RAF inhibitor.

In certain embodiments, the histone deacetylation inhibitor is Vorinastat.

In certain embodiments, the MAPK pathway inhibitor is a MEK inhibitor.

In certain embodiments, the MEK inhibitor is cobimetinib or trametinib.

In certain embodiments, the radiolabeled conjugate is administered in a single dose.

In certain embodiments, the radiolabeled conjugate is administered in multiple doses.

In certain embodiments, the radiolabeled conjugate is administered sequentially daily for several days.

In certain embodiments, the radiolabeled conjugate is administered once per week for 1 month.

In certain embodiments, the radiolabeled conjugate is administered once per week for up to 6 months.

In certain embodiments, the radiolabeled conjugate is administered in a dose of 1 mCi for medical imaging.

In certain embodiments, the radiolabeled conjugate is administered in a dose of up to mCi for medical imaging.

In certain embodiments, the radiolabeled conjugate is administered in a dose of up to 50 mCi for medical imaging.

In certain embodiments, the radiolabeled conjugate is administered in a dose of 0.1 mCi for medical treatment of the cancerous tumors.

In certain embodiments, the radiolabeled conjugate is administered in a dose of up to 1 mCi for medical treatment of the cancerous tumors.

In certain embodiments, the radiolabeled conjugate is administered in a dose of up to 10 mCi for medical treatment of the cancerous tumors.

In certain embodiments, the radiolabeled conjugate is administered in a dose of up to 100 mCi for medical treatment of the cancerous tumors.

In certain embodiments, the conjugate is administered for more than a month.

In certain embodiments, the conjugate is administered for more than a year.

In certain embodiments, the radiolabeled conjugate is administered at a dosage of at least 1500 mg/day.

In certain embodiments, the present invention provides a method of treating drug-resistant melanoma, comprising administering the conjugate as described above to a patient in need thereof.

In certain embodiments, the melanoma is resistant to vemurafenib treatment.

In certain embodiments, the present invention provides a combination of a) an agent that increases expression of MCR1, and b) the conjugate as described above for the prophylactic or therapeutic treatment of hyperproliferative disorder.

In certain embodiments, the hyperproliferative disorder is melanoma.

In certain embodiments, the combination provides a synergistic effect in treating the hyperproliferative disorder.

In certain embodiments, the agent that increases expression of MCR1 is vemurafenib, PBA, a histone deacetylation inhibitor and/or another MEK inhibitor.

In certain embodiments, the agent that increases expression of MCR1 is vemurafenib.

In certain embodiments, the agent that increases expression of MCR1 is PBA.

In certain embodiments, the present invention provides a method of treating melanoma in a patient in need thereof that has received treatment with vemurafenib, PBA, a histone deacetylation inhibitor and/or another MEK inhibitor or other MAPK pathway inhibitor, comprising administering an agent that increases expression of MCR1 in combination with the conjugate as described above to the patient.

In certain embodiments, the method further comprises administering one or more immune checkpoint inhibitors (ICIs).

In certain embodiments, the ICI comprises a CLTA-4 inhibitor, a PD-1inhibitor and/or a PD-L1 inhibitor.

In certain embodiments, the ICI is the CLTA-4 inhibitor ipilimumab (Yervoy).

In certain embodiments, the ICI is the PD-1 inhibitor pembrolizumab (Keytruda) and/or nivolumab (Opdivo).

In certain embodiments, the ICI is the PD-L1 inhibitor atezolizumab (Tecentriq).

In certain embodiments, the present invention provides a kit comprising an agent that increases expression of MCR1, the conjugate of as described above, a container, and a package insert or label indicating the administration of the agent with the conjugate as described above for treating a hyperproliferative disorder.

### BRIEF DESCRIPTION OF THE DRAWINGS

FIG. 1: Flowcytometry analysis of MCR1 expression in A375 malignant melanoma cells. A375 cells were treated with either 2 μmole Vemurafenib or 3 mmole 4-PBA for 4 h and 24 h (n=3). The treated and untreated (control) cells were stained with anti-MC1R-phycoerythrin (PE) monoclonal antibody conjugate. Fluorescence intensity was corrected by auto-fluorescence of cells without staining and data were expressed as relative (vs control) fluorescence intensity±SD. Statistical significance was determined by Student's T-test (*P<0.05; **P<0.01; ***P<0.001; ****P<0.0001).

FIG. 2: Flowcytometry analysis of MCR1 expression in A2058 malignant melanoma cells. A2058 cells were treated with either 2 μmole Vemurafenib or 3 mmole 4-PBA for 4 h and 24 h (n=3). The treated and untreated (control) cells were stained with anti-MC1R-phycoerythrin (PE) monoclonal antibody conjugate. Fluorescence intensity was corrected by auto-fluorescence of cells without staining and data were expressed as relative (vs control) fluorescence intensity±SD. Statistical significance was determined by Student's T-test (*P<0.05; **P<0.01; ***P<0.001; ****P<0.0001).

FIG. 3: Flowcytometry analysis of MCR1 expression in SK-Mel3 malignant melanoma cells. SK-Mel3 cells were treated with either 2 μmole Vemrafenib or 3 mmole 4-PBA for 4 h and 24 h (n=3). The treated and untreated (control) cells were stained with anti-MC1R-phycoerythrin (PE) monoclonal antibody conjugate. Fluorescence intensity was corrected by auto-fluorescence of cells without staining and data were expressed as relative (vs control) fluorescence intensity±SD. Statistical significance was determined by Student's T-test (*P<0.05; **P<0.01; ***P<0.001; ****P<0.0001, N.S. non-significant).

FIG. 4: Typical radio-HPLC chromatogram of co-injection of DOTA-PEG4-VMT-MCR1 and [²⁰³Pb]DOTA-PEG4-VMT-MCR1. (VMT=Viewpoint Molecular Targeting)

FIG. 5: Two hour internalization study of linker modified variants.

FIGS. 6A-6D: Pharmacokinetics characteristics of [203Pb]DOTA-VMT-MCR1 and [203Pb]DOTA-PEG4-VMT-MCR1 in B16/F1 murine melanoma-bearing C57 mice. Left bar of each organ or time-point measured ((FIG. 6A) 1 h, (FIG. 6B) 4 h, (FIG. 6C) 24 h, (FIG. 6D) urine sample) usage of Pb-203 DOTA-VMT-MCR1; Right bar of each organ or time-point measured usage of Pb-203 DOTA-PEG4-VMT-MCR1.

FIG. 7. [Pb-212]DOTA-PEG4-VMT-MCR1 improved therapy for metastatic melanoma tumors in mice compared to standard of care BRAFᵢ. Mice bearing A2058 tumor xenografts were administered with vehicle (CTRL); 10 mg/kg Vemurafenib (BRAFi) twice a day (VEM); i.p. injected 60 mg/kg 4-PBA (PBA); i.v. injected 120 μCi of [²¹²Pb]DOTA-VMT-MCR1 in 3 fractions over 6 days (PB-212); or the combinations (VEM/PBA, VEM/PB-212 and VEM/PBA/VEM 212). Average tumor volumes with SDs were determined from 9-10 animals per group. Experiments conducted according to animal protocols approved by the University of Iowa Animal Care and Use Committee (IACUC).

FIG. 8. [Pb-212]DOTA-PEG4-VMT-MCR1 therapy for metastatic melanoma tumors in mice improved survival compared to standard of care BRAFᵢ. Mice bearing A2058 human melanoma tumor xenografts were administered with vehicle (CTRL); 10 mg/kg Vemurafenib (BRAFi) twice a day (VEM); i.p. injected 60 mg/kg 4-phenylbutyrate (PBA); i.v. injected 120 μCi of [²¹²Pb]DOTA-VMT-MCR1 in 3 fractions over 6 days (PB-212); or the combinations (VEM/PBA, VEM/PB-212 and VEM/PBA/VEM 212). Animal were euthanized when tumor size reached 1500 mm³, loss of 30% body weight; or in case of ruptured tumor ulceration. Experiments conducted according to animal protocols approved by the University of Iowa Animal Care and Use Committee (IACUC).

FIG. 9. Examples of linkers: (1) polyethyleneglycol (PEG)-based linkers with 2, 4, and 8 PEG subunits; (2) aliphatic (ALP) linkers of 2 and 4 carbons; and a piperidine (PIP) based linker with mixed characteristics.

FIG. 10. Real-time PCR analysis of MC1R expression in A2058 and MEWO melanoma cells. A2058 cells were exposed to BRAFᵢ GSK2118436, MEKᵢ GSK1120212 and HDACᵢ PBA for 24 h. Similarly, MEWO cells were treated with PBA and SAHA. Total RNA was isolated and reverse transcripted to cDNA. Real-time PCR was performed using 50 ng of each cDNA sample with labeled primer VIC-MC1R and FAM-NADPH. mRNA fold changes were calculated by ΔΔCt method and expressed as (n=3; Mean±SEM). These data demonstrate that the expression of MCR1 can be pharmacologically enhanced in human melanoma cells.

FIGS. 11A-11D. MCR1 expression can be enhanced in human melanoma cells by incubation with PBA and FDA approved melanoma drugs. (FIGS. 11A-11B) Flow cytometry analysis of MC1R expression in human melanoma cells: (FIG. 11A) SK-MEL3 BRAF^{V600E}; (FIG. 11B) A2058 BRAF^{V600E}; incubated for 4 hours with clinically-relevant concentrations of BRAFᵢ: PLX4032 (10 μM), GSK2118436 (2 μM); MEKᵢ: GDC0973 (2 μM), GSK1120212 (2 μM); 4-PBA (4 mM). Cells were stained with anti-MC1R-phycoerythrin (PE) monoclonal antibody conjugate. Fluorescence intensity was corrected by PE-conjugated isotype. Data were expressed as normalized mean fluorescence intensity (NMFI; n=4±SD; *p<0.05). (FIGS. 11C-11D) PBA-enhanced MCR1 expression increases binding of MCR1-specific peptides. (FIG. 11C) SK-MEL3 BRAF^{V600E}; and (FIG. 11D) A2058 BRAF^{V600E} was confirmed by radiobinding assay using MCR1-specific peptide [¹²⁵I]-NDP-a-MSH following incubation for 4 hours with PBA and FDA approved drugs (as in FIG. 10). Following incubation, media was changed and cells were incubated with NDP-a-MSH for 30 min. Binding expressed as radioactivity bound relative to untreated controls (same cells) (n=4+SD; *p<0.05). These data support the hypothesis that BRAFᵢ and PBA can be used to enhance expression of MCR1—and binding of MCR1-targeted peptides to melanoma cells.

FIGS. 12A-12D. Parameters that reflect mitochondrial/cellular oxidative state; ER stress; and autophagy were monitored with the acquisition of resistance to BRAFᵢ in A375 BRAFᵢ-sensitive melanoma cells. (FIG. 12A) BRAFᵢ results in an initial increase cellular oxidative state, but decreases as cells develop resistance; (FIG. 12B) A similar pattern is observed in mitochondrial reactive oxygen species as measured by Mitosox ROS probe; (FIG. 12C) Similarly, ER-stress increases as cells develop resistance as measured by ER-stress marker GRP78 protein expression by flow cytometry; and (FIG. 12D) Transmission electron microscopy (TEM) was used to detect and quantify autophagy. TEM enables differentiating between lysosomes, autophagosomes, and autolysosomes. BRAFᵢ-resistant A375VR cells (which had been clonogenically selected over 1 month of BRAFᵢ treatment) and BRAFᵢ-sensitive A375 cells were fixed overnight and en bloc stained with uranyl acetate; and quantified by standard grid-based-blinded criteria. A significant increase in the level of autophagy was observed in BRAFᵢ-resistant (A375VR) cells relative to BRAFᵢ sensitive cells. These results support the hypothesis that acquisition of BRAFᵢ-resistance is caused by metabolic rewiring that leads to increased ER stress and autophagy that could be mediated by oxidative stress (n=3; ***p<0.001).

FIGS. 13A-13B. PBA treatment promotes cell death of BRAFᵢ-resistant metastatic melanoma and upregulates MCR1 expression in a time dpe. (FIG. 13A) BRAFᵢ-resistant A375VR cells were incubated with 5 μM vemurafenib in combination with ER-stress-relieving drug 4-PBA for up to 6 days. No change in clonogenic survival was observed for BRAFᵢ-resistant A375VR cells in the absence of PBA. However, nearly 90% clonogenic cell death was observed for BRAFᵢ-resistant A375VR treated in combination with doses of PBA as low as 500 μM. (FIG. 13B) Flow cytometry analysis of MCR1 expression in A2058 and A375 malignant melanoma cells. Cells were treated with 3 mmole 4-PBA for 4 h and 24 h (n=3). The treated and untreated (control) cells were stained with anti-MC1R-phycoerythrin (PE) monoclonal antibody conjugate. Fluorescence intensity was corrected by auto-fluorescence of cells without staining and data were expressed as relative fluorescence intensity (IF) (vs. control)±SD. These data support the hypothesis that PBA can serve the dual purpose of sensitizing BRAFᵢ-resistant metastatic melanoma and upregulating MCR1 expression to enhance MCR1-RT.

FIGS. 14A-14B. (FIG. 14A) Co-administration (i.v. or i.p.) of PBA (120 mg/kg) significantly blocked kidney uptake, but did not affect tumor accumulation of [²⁰³Pb]DOTA-MCR1 (0.037 MBq) at 2 h p.i. in mice; Tumor and kidneys were harvested, weighed and assayed by NaI detector. Results are ID %/g tissue±SD; **P<0.01; ****P<0.0001. (FIG. 14B Right) Pre-administered of PBA (i.p. 60 mg/kg) and BRAFᵢ (vemurafenib; p.o. 5 mg/kg) 3 h prior to tail vein injection of 13 MBq [²⁰³Pb]DOTA-MCR1 in human (A2058) melanoma-bearing athymic nu/nu mice (SPECT image 1 h p.i.); (FIG. 14B Left) An identical animal was administered an identical dose of [²⁰³Pb]DOTA-MCR1 without PBA/BRAFᵢ (saline). Images were identically processed and analyzed using Inveon Workplace software (identical contrast, intensity, etc.). These data support the hypothesis that combining MCR1-RT with PBA that enhance MCR1-RT by reducing kidney uptake and improving tumor uptake of MCR1-peptides.

FIGS. 15A-15C. Survival of mice bearing human metastatic melanoma xenografts treated with a single dose (i.v.) of [²¹²Pb]DOTA-MCR1, shown as ²¹²Pb (100-140 μCi) with and without BRAFᵢ (vemurafenib 10 mg/kg b.i.d); PBA (120 mg/kg i.p.); and combinations. BRAFᵢ(p.o.) and PBA (i.p.) treatments were administered 3 h prior to injection of [²¹²Pb]DOTA-MCR1 and were continued daily for the duration of experiments. Treatments were standardized to begin when tumors reach 100 mm³. Mice were euthanized according to IACUC protocols (when tumors reached 1500 mm³ or ulceration appeared) or at about 100 d. Three possible complete responses to [²¹²Pb]DOTA-MCR1 were observed, pending autopsy. These data support the hypothesis that [²¹²Pb]DOTA-MCR1 therapy has the potential to improve outcomes for metastatic melanoma patients relative to standard of care therapy.

FIGS. 16A-16D. Pb-specific chelator (PSC) improves radiolabeling of peptides and does not interfere with binding of peptides to receptors. (FIG. 16A) DOTA and TCMC have been proposed for Pb labeling, but result in a residual charge, while the PSC is charge neutral; (FIG. 16B) RadioHPLC trace of [²⁰³Pb]PSC-MCR1 peptide showing >99% radiochemical purity (RCP); (FIG. 16C) Rate of incorporation of ²¹²Pb monitored at 37° C., 60° C., and 90° C. (pH 5.5 buffer); % incorporation measured by iTLC of a PSC-MCR1 peptide. Radiolabeling efficiency was nearly 90% in 10 min. at 37° C. vs. <58% for the DOTA conjugate. (FIG. 16D) Competitive binding assays (B16 melanoma cells expressing MCR1) showed a slightly higher binding affinity for the PCS-MCR1 conjugate compared to the DOTA-MCR1 conjugate.

FIG. 17. Structures of the DOTA-C-MCR1 and PSC-C-MCR1 click cyclized peptides to be used for the proposed investigation. These peptides are synthesized by standard protocols using an automated peptide synthesis module and prep-HPLC purification systems. The click cyclized variants have excellent tumor targeting characteristics.

FIGS. 18A-18B. Comparison of kidney pathology analysis of (FIG. 18A) control and (FIG. 18B) kidney tissue 3 months following injection of a 100 μCi dose of [²¹²Pb]DOTA-MCR1. This study was conducted using the Re-cyclized MCR1 peptide and no effort was made to block kidney uptake of the radiopeptide. In FIG. 18B, moderate to marked, multifocal interstitial inflammation surrounding the vessels at the corticomedullary junction composed primarily of plasma cells with fewer lymphocytes (white arrows) is observed; clusters of renal tubules which appear mildly dilated and are lined by flattened epithelial cells (black arrows), some of which have very large nuclei compared to others (likely regeneration). Multifocal, scattered glomerular capillary loops are smudged and almost acellular than usual (glomerular sclerosis) (encircled). Analysis by co-investigators Gibson-Corely and Zepeda-Orozco at the University of Iowa. Importantly, the peptide proposed in the current application (click-cyclized) reduces kidney accumulation (no blocking) by 3-fold and improves tumor:kidney ratio 7-fold.

FIG. 19. Representative tumor growth curve for human melanoma tumor bearing mice treated with 100 μCi (3.7MBq) of [²¹²Pb]DOTA-MCR1 alone (²¹²Pb) and combined with BRAFᵢ (²¹²Pb/PLX4032); PBA (²¹²Pb/PBA) and a triple combination (²¹²Pb/PLX4032/PBA) relative to untreated controls. Tumor size of untreated controls reached IACUC protocol limits (1500 mm³) by 15 days post study initiation (at 100 mm³ tumor size). The most pronounced tumor response was observed in the triple combination treatment group (n=7, mean±SEM).

FIGS. 20A-20B. GRP78 analysis (FIG. 20A. GRP78 IHC (10×) image; FIG. 20B. GRP78 microscopy (20×) image) of kidney and tumor PE samples are used to examine the role of ER stress in the fibrogenesis in kidney tubules and in tumor response with and without the inclusion of PBA, which is known to relieve ER stress. See also FIGS. 12A-12D for the potential role of ER stress in the development of resistance in melanoma.

FIG. 21. Survival of mice bearing human metastatic melanoma xenografts (A375) treated with a single dose (i.v.) of [²¹²Pb]DOTA-MCR1, shown as ²¹²Pb (˜100 μCi) with and without a combination of BRAFᵢ (vemurafenib 10 mg/kg b.i.d); PBA (120 mg/kg i.p.); and hydroxychloroquine. Treatments were standardized to begin when tumors reach 100 mm³. Mice were euthanized according to IACUC protocols (when tumors reached 1500 mm³ or ulceration appeared) or at about 100 d. These data support the hypothesis that [²¹²Pb]DOTA-MCR1 therapy has the potential to improve outcomes for metastatic melanoma patients relative to standard of care therapy.

FIGS. 22A-22C. FIG. 22A provides the structure of VMT1, FIG. 22B provides the structure of VMT2, and FIG. 22C provides the structure of PSC-PEG-CLICK.

FIGS. 23A-23C. FIGS. 23A-23C illustrate MC1R-targeted peptide ligand direct ionizing radiation to melanoma via binding with the receptor. FIG. 23A illustrates competitive binding of VMT01 and [^{nat}Pb]VMT01 against [¹²³I]NDP-α-MSH in B16-F10 cells; FIG. 23B illustrates the biodistribution of [²⁰³Pb]VMT01 in athymic nu/nu bearing B16-F10 melanoma (n=2 male and n=2 female at each time point); Data were expressed as percent of injected dose per gram of tissue (% ID/g)±SD; FIG. 23C shows the structure of VMT01.

FIGS. 24A-25D. FIGS. 24A-25D illustrate the anti-tumor effect from combination of single dose [²¹²Pb]VMT01 α-TRT and ICIs B16-F10 melanoma. FIG. 24A illustrates individual tumor volume in animals after treatments of rat IgG isotype control (control), single dose of 4.1 MBq [²¹²Pb]VMT01; 200 μg of anti CTLA-4+200 μg anti PD-1 (ICIs), and combination of [²¹²Pb]VMT01 and ICIs (n=7 in each group); FIG. 24B illustrates overall fractional survival in C57BL6 mice bearing B16-F10 melanoma that received rat IgG isotype control, [²¹²Pb]VMT01, ICIs, and combination of [²¹²Pb]VMT01 and ICIs (n=7 in each group); statistic analysis was performed using Gehan-Breslow-Wilcoxon test: *** p<0.001; FIG. 24C illustrates individual tumor growth of B16-F10 tumor re-challenge in mice with complete response to combination of [²¹²Pb]VMT01 and ICIs; FIG. 24D illustrates mice rejected further implantation of B16-F10 melanoma tumor up to 60 days after re-challenge.

FIGS. 25A-25B. FIGS. 25A-25B illustrate the fractionated dose of [²¹²Pb]VMT01 α-TRT in combination with ICIs in C57BL6 mice bearing B16-F10 melanoma. FIG. 25A illustrates individual tumor volume in each group of animals after treatments were initiated. Treatments included rat IgG isotype control (control), fractionated 4 MBq [²¹²Pb]VMT01 (FRT [²¹²Pb]VMT01); 200 μg of anti CTLA-4+200 μg anti PD-1 (ICIs), and combination of FRT [²¹²Pb]VMT01 and ICIs (n=7 in each group); FIG. 25B illustrates overall fractional survival in B16-F10 tumor xenograft models that received control IgG, ICIs, FRT [²¹²Pb]VMT01 and combination of FRT [²¹²Pb]VMT01 and ICIs; statistical analysis was performed using Gehan-Breslow-Wilcoxon test: ** p<0.01; *** p<0.001.

FIGS. 26A-26D. FIGS. 26A-26D illustrate [²¹²Pb]VMT01 α-TRT induces anti-tumor immune responses that rely on adaptive immunity. FIG. 26A illustrates individual tumor volume in RAG1 K/O mice after treatments of rat IgG isotype control (control), single dose of 4.1 MBq [²¹²Pb]VMT01; 200 μg of anti CTLA-4+200 μg anti PD-1 (ICIs), and combination of [²¹²Pb]VMT01 and ICIs (n=7 in each group); FIG. 26B illustrates overall fractional survival in RAG1 K/O mice bearing B16-F10 melanoma that received IgG control, [²¹²Pb]VMT01, ICIs, and combination of [²¹²Pb]VMT01 and ICIs (n=7 in each group); statistical analysis was performed using Gehan-Breslow-Wilcoxon test: n.s. non-significant; *** p<0.001; cell based vaccine using [²¹²Pb]VMT01 treated B16F0 or B16-F10 cells was injected in C57BL6 mice (n=7). Individual tumor growth of re-challenging (FIG. 26C) and (FIG. 26D) B16-F0 tumor on the contralateral side of the primary tumor.

FIGS. 27A-27B. FIGS. 27A-27B illustrate [²¹²Pb]VMT01 sensitizes immunotolerant melanoma cells to ICIs treatment. [²¹²Pb]VMT01 treated (FIG. 27A) B16-PR tumor and (FIG. 27B) YUMM-PR melanoma responded to ICIs treatment in C57BL6 mice. Arrows indicated ICIs treatment. ** p<0.01, *** p<0.001.

FIG. 28A-28B. FIG. 28A-28B illustrate [²¹²Pb]VMT01 enhances tumor infiltrating lymphocytes in B16-F10 melanoma. FIG. 28A illustrates lymphocytes were gated to exclude non-lymphocyte populations based on forward and side scatter (P5C and 5SC) and stained for live-dead discriminator, CD45, CD19, CD3, CD4, and CD8a; FIG. 28B illustrates FACS analysis of CD45, CD19, CD3, CD4, and CD8a lymphocytes in control for control (n=5) vs. ²¹²Pb α-therapy (n=4) treated B16-F10 tumor. Statistical analysis: n.s. non-significant; *p<0.05; **p<0.01.

### DETAILED DESCRIPTION

The melanocortin-1 receptor (MCR1) is a G-protein coupled receptor (GPCR) that belongs to melanocortin receptor family. There are five melanocortin receptors that have been isolated and cloned to date. A discussion of the melanocortin receptors is discussed in US Patent Publication 2014/0238390, which is incorporated by reference herein. MCR1 is found in a number of different cell lines and tissues, though it has only been found in high levels in melanocytic cells. MCR1 has a role in regulating skin pigmentation. MCR1 is over-expressed in most murine and human melanoma metastases.

Alpha-melanocyte stimulating hormone (α-MSH) signals via the MC1R in melanocytes to stimulate eumelanogenesis (the formation of the black pigment eumelanin) via upregulation of the enzyme tyrosinase and via melanocyte proliferation. A variety of peptides, peptide derivatives, peptidomimetics and small molecules that bind to and activate or inhibit the MCR1 have been reported.

Melanoma is a dangerous type of skin cancer that develops in cells that produce melanin (melanocytes), usually presenting as an irregular spot/mole on the skin. Causes of melanoma include UV radiation and a genetic predisposition to this type of cancer. Unlike other cancers, prevalence of melanoma is increasing, with the highest occurrence among individuals 25-29 years old. The overall lifetime risk of developing melanoma is 2.4%. In 2015, 73,870 new invasive melanomas are expected to be diagnosed, with 9,940 people expected to die of melanoma. With early treatment, survival rate is 97%.

Melanoma can migrate to other parts of the body (metastatic melanoma), and one year survival rate drastically decreases with metastasis—15-20% for Stage IV. Current types of treatment include surgery, immunotherapy (Immune checkpoint inhibitors for advanced melanoma), chemotherapy, radiation therapy, targeted therapy (target cells with gene changes) and BRAF Inhibitors. BRAF is a protein kinase of the mitogen-activated protein kinase (MAPK) pathway, and it regulates cell growth, proliferation, and differentiation. Research suggests a BRAF^{V600E} mutation causes the BRAF protein (produced through the MAPK pathway) to become oncogenic. The mutation may lead to increased and uncontrolled cell proliferation, and resistance to apoptosis. The BRAF mutation is observed in about 50% of melanoma tumors. Its presence is associated with poor prognosis in metastatic melanoma.

Melanoma is the fastest growing cancer incidence in the United States. Surgery is curative for melanoma confined to the skin, but metastatic melanoma is lethal. Current FDA approved therapies for metastatic melanoma (e.g., Vemurafenib, Ipilimumab), have increased life expectancy by months, however resistance develops rapidly. The exact mechanism by which drug resistance develops is unclear; however, autophagy is known to play a major role. Autophagy is a self-degradative response of the cell towards nutrient stress. Conversely, autophagy also plays a housekeeping role by removing mis-folded or aggregated proteins and clearing damaged organelles by forming autophagosomes. Thus, autophagy is believed to play an important role in tumor progression and developing drug resistance during later stages of cancer. The Unfolded Protein Response (UPR) mediated by GRP78 ER associated protein degradation is one of the pathways that initiate autophagy in stressed cells. UPR involves the activation of three signaling pathways mediated by IRE-1, PERK and ATF6. These pathways work towards decreasing the protein load of ER by increasing the expression of molecular chaperons, activation of ERAD (ER associated protein degradation) and autophagy. However if the damage caused by the stress is extensive UPR signaling pathways initiate apoptosis. Amy S. Lee, Cancer Res (2007); 77:3496-3499. Emerging evidence shows that in malignant cells ER stress can be pro-survival and contribute to the development of drug resistance by initiating autophagy.

Interestingly, initial responses (tumor shrinkage) are also very common among patients treated with Vemurafenib (Zelboraf®). Vemurafenib targets a gene mutation in metastatic melanoma called BRAF-V600E, which causes metastatic melanoma cells to divide and proliferate uncontrollably and rapidly. Thus, when patients are treated with Vemurafenib, there is a generally very positive response. However, a small but lethal subpopulation of cells becomes resistant to the treatment. Thus, patients appear to be virtually cured, but the small subpopulations of cells that are resistant to treatment eventually (within months) begin to divide and proliferate rapidly and tumors regrow at precisely the same locations.

Over 80% of malignant melanomas express high levels of the melanocyte stimulating hormone (α-MSH) receptor, melanocortin 1 receptor (MCR1, also called MC1R). In certain embodiments, the present therapy provides a radiolabeled peptide that binds with high affinity and specificity to MC1R and delivers radiation precisely to melanoma cells. This discovery means that the present treatment is especially effective in killing cells that have become resistant to Vemurafenib, making the combination of Vemurafenib with the current therapy an exciting new treatment for metastatic melanoma. A new combination therapy has been developed that kills vemurafenib resistant cells, but is virtually non-toxic to the rest of the body.

### MCR1 Ligands

In certain embodiments, the MCR1 ligand is a targeting peptide that is an alpha-melanocyte stimulating hormone (α-MSH). Alpha-MSH is a tridecapeptide (Ser-Tyr-Ser-Met-Glu-His-Phe-Arg-Trp-Gly-Lys-Pro-Val-NH₂) (SYSMEHFRWGKPV) that regulates skin pigmentation in vertebrates. The core α-MSH sequence, His-Phe-Arg-Trp, has been found to be sufficient for receptor recognition. α-MSH specifically recognizes melanotropin receptors. Various synthetic α-melanotropin analogs have been prepared and characterized for α-melanotropin activity. (V. J. Hruby, et al., Design, Synthesis and Conformation of Superpotent and Prolonged Acting Melanotropins (1993) Annals of the New York Acad. of Sci., 680: 51-63.) They reported that cyclic analogs of α-MSH (as described by U.S. Pat. No. 4,485,039) display properties that increase their potency toward the α-MSH receptor, prolong their activity and increase their resistance to in vivo enzymatic degradation.

According to the present invention, there is provided a compound for use as a diagnostic or therapeutic pharmaceutical consisting essentially of an α-MSH analog that has an integrally located a radionuclide. The radiolabeled alpha-melanotropin is administered to the patient in an amount sufficient to allow uptake and retention by the tumor cells. Examples of suitable MCR1 targeting peptides include those described in U.S. Pat. Nos. 6,338,834; 6,607,709; and 6,680,045; US Patent Publication Nos. 20160046688; 20150284431; 20150119341; 20150038434; 20140128380; and 20140112873, which are incorporated by reference in their entirety herein. In certain embodiments, the α-MSH is linear. In certain embodiments, the α-MSH is cyclic.

In one embodiment, the phrase “selectively binds” means that a compound or polypeptide made or used in the present invention preferentially binds to one type of receptor over another type of receptor when in the presence of a mixture of two or more receptors (e.g., melanocortin receptors, MC1, MC2, MC3, MC4, MC5 receptors).

“Amino acid” or “amino acid sequence” include an oligopeptide, peptide, polypeptide, or protein sequence, or to a fragment, portion, or subunit of any of these, and to naturally occurring or synthetic molecules. The terms “polypeptide” and “protein” include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. Capitalized, single-letter abbreviations of the amino acids refer to the natural L-isomer. Lower case, single-letter abbreviations of the amino acids denotes the D-isomer.

The terms “polypeptide,” “peptide,” and “protein” are used interchangeably to refer to polymers of amino acids of any length. Peptides and polypeptides can be either entirely composed of synthetic, non-natural analogues of amino acids, or, is a chimeric molecule of partly natural peptide amino acids and partly non-natural analogs of amino acids. In one aspect, a polypeptide is used in a composition, cell system or process of the invention (e.g., a host cell having a plasmid expressing at least one enzyme of the invention). In addition, polypeptide can refer to compounds comprised of polymers of amino acids covalently attached to another functional group (e.g., solubilizing group, a targeting group, PEG, non-amino acid group, or other therapeutic agent).

Amino acids may be abbreviated using the following designation in parentheses: Proline (Pro), Valine (Val), Lysine (Lys), Omithine (Orn), Norleucine (Nle), Glycine (Gly), Tryptophan (Trp), Alanine (Ala), Phenylalanine (Phe), Arginine (Arg), Histidine (His), Glutamic acid (Glu), Aspartic acid (Asp), Serine (Ser), Methionine (Met), Isoleucine (Ile), Tyrosine (Tyr), Cyclohexylalanine (Cha), 4-fluoro-D-phenylglycine (4-fluoro-D-Phg), 2-thienyl-D-alanine (D-Thi).

Polypeptide compositions of the invention can contain any combination of non-natural structural components. Individual peptide residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2- for —C(═O)—NH—), aminomethylene (CH2-NH), ethylene, olefin (CH═CH), ether (CH2-O), thioether (CH2-S), tetrazole, thiazole, retroamide, thioamide, or ester (see, e.g., Spatola (1983) in Chemistry and Biochemistry of Amino Acids, Peptides and Proteins, Vol. 7, pp. 267-357, “Peptide Backbone Modifications,” Marcel Dekker, N.Y., incorporated herein by reference).

Polypeptides used to practice the method of the invention can be modified by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl terminus. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, PEGylation, proteolytic processing, phosphorylation, prenylation, selenoylation, sulfation, and transfer-RNA mediated addition of amino acids to protein such as arginylation. See, e.g., Creighton, T. E., Proteins—Structure and Molecular Properties 2nd Ed., W. H. Freeman and Company, New York (1993); Posttranslational Covalent Modification of Proteins, B. C. Johnson, Ed., Academic Press, New York, pp. 1-12 (1983), incorporated herein by reference.

“Biologically active” moieties include a molecule or compound that elicits or modulates a physiological response. In one aspect, a biologically active compound stimulates melanocortin receptors, preferably MC1-receptors.

By “modulate” and “modulation” is meant that the activity of one or more proteins or protein subunits is up regulated or down regulated, such that expression, level, or activity is greater than or less than that observed in the absence of the modulator. For example, the term “modulate” can mean “inhibit” or “stimulate”.

“C-terminal sequence” includes reference to the end of the amino acid chain terminated typically, but not necessarily, by a carboxyl group. The convention for writing peptide sequences is to put the C-terminal end on the right and write the sequence from N- to C-terminus. The C-terminal sequence may comprise 1 to 100 amino acids, preferably 2 to 15 amino acids, and even more preferably 3 to 10 amino acids. The C-terminal sequence may terminate with a carboxyl group or the terminus may be modified by well-known methods in the art to comprise a functional member (e.g. targeting group, retention signal, lipid, and anchor).

### Imaging and Therapeutic Radionuclides

In certain embodiments, the peptide that targets MCR1 is radiolabeled for patient imaging with gallium-68, lead-203, zirconium-89, fluorine-18, technetium-99, carbon-11, indium-111, lutetium-177, copper-64, scandium-44 or other radionuclide radiometals that are suitable for imaging of disease. In certain embodiments, the radionuclide is integral in the peptide that targets MCR1.

In certain embodiments, the peptide that targets MCR1 is radiolabeled for patient therapy with lead-212, gallium-67, rhenium-188, thorium-227, actinium-225, yttrium-90, lutetium-177, actinium-225, astatine-211, radium-223, radium-224, or other radionuclide radiometals that emit a particle that is suitable for therapy of disease. In certain embodiments, the radionuclide is integral in the peptide that targets MCR1.

Isotopically-labeled peptides can generally be prepared by conventional techniques known to those skilled in the art. See, e.g., US Patent Publication No. 2014/0128380.

### Chelating Agents

In certain embodiments, the chelating agent is DOTA or other chelator that is used to bind the radionuclide for diagnostic imaging or therapy for cancer or other disease.

In certain embodiments, the chelating agent is DTPA or other chelator that is used to bind the radionuclide for diagnostic imaging or therapy for cancer or other disease.

In certain embodiments, the chelator is based on S-2-(4-Nitrobenzyl)-1,4,7,10-tetraazacyclododecane or other variation on this cyclododecane.

In certain embodiments, the chelator is based on 1,4,7,10-Tetraazacyclododecane-1,4,7-tri(carbamoylmethyl)-10-acetic acid.

In certain embodiments, the chelator is based on S-2-(4-Nitrobenzyl)-1,4,7,10-tetraazacyclododecane tetraacetic acid.

In certain embodiments, the chelator is based on S-2-(4-Aminobenzyl)-1,4,7,10-tetraazacyclododecane tetraacetic acid.

In certain embodiments, the chelator is based on S-2-(4-Aminobenzyl)-1,4,7,10-tetraazacyclododecane tetra-tert-butylacetate.

In certain embodiments, the chelator is based on S-2-(4-Isothiocyanatobenzyl)-1,4,7,10-tetraazacyclododecane tetraacetic acid.

In certain embodiments, the chelator is based on 1,4,7,10-Tetraazacyclododecane-1,4,7-tris-tert-butyl acetate-10-acetic acid.

In certain embodiments, the chelator is based on 1,4,7,10-Tetraazacyclododecane-1,4,7-tris-tert-butyl acetate-10-succinimidyl acetate.

In certain embodiments, the chelator is based on 1,4,7,10-Tetraazacyclododecane-1,4,7-tris-tert-butyl acetate-10-maleimidoethylacetamide.

In certain embodiments, the chelator is based on 1,4,7,10-Tetraazacyclododecane-1,4,7-tris-acetic acid-10-maleimidoethylacetamide.

In certain embodiments, the chelator is based on 1,4,7,10-Tetraazacyclododecane-1,4,7-tris-tert-butyl acetate-10-(N-a-Fmoc-N-e-acetamido-L-lysine).

In certain embodiments, the chelator is based on 1,4,7,10-Tetraazacyclododecane-1,4,7-tris(t-butyl acetate)-10-(3-butynylacetamide).

In certain embodiments, the chelator is based on 1,4,7,10-Tetraazacyclododecane-1,4,7-tris(t-butyl-acetate)-10-(aminoethyl-acetamide).

In certain embodiments, the chelator is based on 1,4,7,10-Tetraazacyclododecane-1,4,7-tris-tert-butyl acetate-10-(azidopropyl ethylacetamide).

In certain embodiments, the chelator is based on 1,4,7,10-Tetraazacyclododecane-1,4,7-tris(t-butyl acetate)-10-(4-aminobutyl)acetamide.

In certain embodiments, the chelator is based on 1,4,7,10-Tetraazacyclododecane-1,4,7,10-tetraacetic acid mono-N-hydroxysuccinimide ester.

In certain embodiments, the chelator is based on 1,4,7,10-Tetraazacyclododecane-1,4,7-tris(acetic acid)-10-(2-thioethyl)acetamide or other variation of DOTA.

In certain embodiments, the chelator is based on S-2-(4-Aminobenzyl)-diethylenetriamine pentaacetic acid or other variation of DTPA.

In certain embodiments, the chelator is based on 3,6,9,15-Tetraazabicyclo[9.3.1]pentadeca-1(15),11,13-triene-4-S-(4-aminobenzyl)-3,6,9-triacetic acid or other variation on this pentadeca macrocycle.

In certain embodiments, the chelator is based on 1-Oxa-4,7,10-tetraazacyclododecane-5-S-(4-aminobenzyl)-4,7,10-triacetic acid or other variation on oxo-substituted macrocycle.

In certain embodiments, the chelator is based on 2-S-(4-Isothiocyanatobenzyl)-1,4,7-triazacyclononane-1,4,7-triacetic acid or other variation on this cyclononane.

In certain embodiments, the chelator is based on 1-(4-isothiocyanatophenyl)-3-[6,17-dihydroxy-7,10,18,21-tetraoxo-27-(N-acetylhydroxylamino)-6,11,17, 22-tetraazaheptaeicosine] thiourea or other variation on deferoxamine.

### Linkers

In certain embodiments, L is a chemical linker that is inserted into a position between the peptide backbone that recognizes the MCR1 protein and the chelator that is used to radiolabel the composition using radionuclides for diagnostic imaging and/or therapy; and the linker improves the internalization of the composition into cells and improves the retention of the composition in tumors for more precise delivery of radiation to the cancerous tissue.

In certain embodiments, L is a hydrophobic linker consisting of an aliphatic carbon chain that connects the chelator to the peptide backbone.

In certain embodiments, L is a hydrophilic linker that includes heteroatom substitutions in the aliphatic chain that connects the chelator to the peptide backbone.

In certain embodiments, L is a mixture of hydrophilic and hydrophobic entities including piperidine insertions of amino acid insertions to lengthen the chain and modulate the pharmacodynamics properties of the composition.

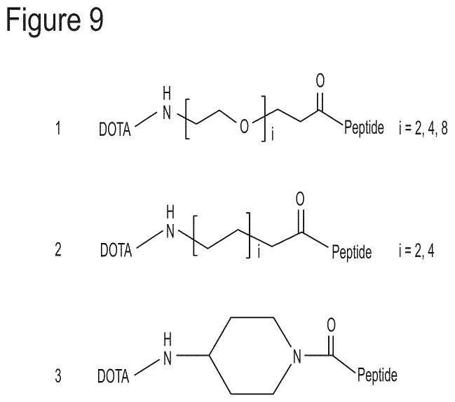

In certain embodiments, L is PEGₙ, wherein n is 1-10, such as 1, 2, 3, 4, 5, 6, 7, 8, 9, or 10. In certain embodiments, n is 2, 4 or 8 PEG subunits. In certain embodiments, n is 4. (FIG. 9)

In certain embodiments, L is an aliphatic (ALP) linker of 2 or 4 carbons. (FIG. 9)

Other linkers are known in the art. See, e.g., Bandari R P, Jiang Z, Reynolds T S, Bernskoetter N E, Szczodroski A F, Bassuner K J, Kirkpatrick D L, Rold T L, Sieckman G L, Hoffman T J, Connors J P, Smith C J. Synthesis and biological evaluation of copper-64 radiolabeled [DUPA-6-Ahx-(NODAGA)-5-Ava-BBN(7-14)NH2], a novel bivalent targeting vector having affinity for two distinct biomarkers (GRPr/PSMA) of prostate cancer. Nucl Med Biol. 2014; 41(4):355-363. doi: 10.1016/j.nucmedbio.2014.01.001. PubMed PMID: 24508213; PMCID:PMC4041584; Dumont R A, Tamma M, Braun F, Borkowski S, Reubi J C, Maecke H, Weber W A, Mansi R. Targeted radiotherapy of prostate cancer with a gastrin-releasing peptide receptor antagonist is effective as monotherapy and in combination with rapamycin. J Nucl Med. 2013; 54(5):762-769. doi: 10.2967/jnumed.112.112169. PubMed PMID: 23492884; Gourni E, Mansi R, Jamous M, Waser B, Smerling C, Burian A, Buchegger F, Reubi J C, Maecke H R. N-terminal modifications improve the receptor affinity and pharmacokinetics of radiolabeled peptidic gastrin-releasing peptide receptor antagonists: examples of 68Ga- and 64Cu-labeled peptides for PET imaging. J Nucl Med. 2014; 55(10):1719-1725. doi: 10.2967/jnumed.114.141242. PubMed PMID: 25146125; Jamous M, Tamma M L, Gourni E, Waser B, Reubi J C, Maecke H R, Mansi R. PEG spacers of different length influence the biological profile of bombesin-based radiolabeled antagonists. Nucl Med Biol. 2014; 41(6):464-470. doi: 10.1016/j.nucmedbio.2014.03.014.

PubMed PMID: 24780298; Mansi R, Abiraj K, Wang X, Tamma M L, Gourni E, Cescato R, Berndt S, Reubi J C, Maecke H R. Evaluation of three different families of bombesin receptor radioantagonists for targeted imaging and therapy of gastrin releasing peptide receptor (GRP-R) positive tumors. J Med Chem. 2015; 58(2):682-691. doi: 10.1021/jm5012066. PubMed PMID: 25474596; Pan D, Xu Y P, Yang R H, Wang L, Chen F, Luo S, Yang M, Yan Y. A new (68)Ga-labeled BBN peptide with a hydrophilic linker for GRPR-targeted tumor imaging. Amino Acids. 2014; 46(6):1481-1489. doi: 10.1007/s00726-014-1718-y. PubMed PMID: 24633452; Stott Reynolds T J, Schehr R, Liu D, Xu J, Miao Y, Hoffman T J, Rold T L, Lewis M R, Smith C J. Characterization and evaluation of DOTA-conjugated Bombesin/RGD-antagonists for prostate cancer tumor imaging and therapy. Nucl Med Biol. 2015; 42(2):99-108. doi: 10.1016/j.nucmedbio.2014.10.002. PubMed PMID: 25459113.

In certain embodiments, L is a piperidine (PIP) based linker with mixed characteristics. (FIG. 9)

### Anti-Melanoma Conjugate

In certain embodiments, the present invention provides a melanoma-targeting conjugate comprising Formula I:

T-L-X
wherein T is a radiolabeled MCR1 ligand,
   L is a linker, and
   X an anti-cancer composition,
   for the therapeutic treatment of melanoma.

In certain embodiments, the MCR1 Ligand is an MCR1 Ligand as described above.

In certain embodiments, the linker is a linker as described above.

In certain embodiments, the anti-cancer composition is the chelator-modified PEG4 linked Re-cyclized MCR1 targeted peptide referred to as DOTA-PEG4-VMT-MCR1 or the PEG4-VMT-MCR1 modified to include a different chelator

Agents that Increase Expression of MCR1

Vemurafenib (Zelboraf®) is a B-Raf enzyme inhibitor developed for the treatment of late-stage melanoma. Vemurafenib stops the proliferative effects of oncogenic BRAF protein. The name “vemurafenib” comes from V600E mutated BRAF inhibition.

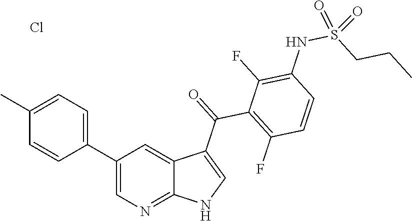 [SMILES: CCCS(=O)(=O)Nc1ccc(F)c(C(=O)c2c[nH]c3ncc(-c4ccc(C)cc4)cc23)c1F ; Cl]

ZELBORAF® Vemurafenib is indicated for the treatment of patients with unresectable or metastatic melanoma with BRAF V600E mutation as detected by an FDA-approved test. Tumor specimens are confirmed for the presence of BRAF V600E mutation prior to initiation of treatment with Vemurafenib. The recommended dose is 960 mg orally twice daily taken approximately 12 hours apart with or without a meal; 720 mg twice daily for first appearance of intolerable Grade 2 or Grade 3 adverse reactions; or 480 mg twice daily for second appearance of Grade 2 (if intolerable) or Grade 3 adverse reactions or for first appearance of Grade 4 adverse reaction (if clinically appropriate). Unfortunately, metastatic melanoma can resist vemurafenib treatment. Vemurafenib slows tumor progression for only about 5.3 months. As a result, finding an effective treatment for metastatic melanoma is challenging.

The term “anti-cancer agent” includes a Triphenylphosphonium (TPP) agent or derivative thereof that increases reactive oxygen species (ROS) levels in cancer cell mitochondria, and a pharmaceutically acceptable diluent or carrier. As used herein, the term triphenylphosphonium (TPP) is any molecule containing a triphenylphosphine cation (⁺PPh₃) moiety. See, e.g., WO 2013/019975 and WO 2014/124384, which are incorporated by reference herein.

TPP salts can be reacted with alcohols, alkyl halides, and carboxylic acids, which allow them to be used as starting materials for the synthesis of a large variety of chemical derivatives, e.g., XTPP agents. Charged molecules generally cannot pass through cell membranes without the assistance of transporter proteins because of the large activation energies need to remove of associated water molecules. In the TPP molecules, however, the charge is distributed across the large lipophilic portion of the phosphonium ion, which significantly lowers this energy requirement, and allows the TPP to pass through lipid membranes. The phosphonium salts accumulate in mitochondria due to the relatively highly negative potential inside the mitochondrial matrix. The compositions of the present invention utilize XTPP agents that have activity in treating cancer cells, in that the XTPP agents preferentially localize to cancer cells, as compared to the comparable normal cells because cancer cells are often characterized by abnormal mitochondrial oxidative metabolism (Aykin-Burns N, Ahmad I M, Zhu Y, Oberley L W, and Spitz D R: Increased levels of superoxide and hydrogen peroxide mediate the differential susceptibility of cancer cells vs. normal cells to glucose deprivation. Biochem. J. 2009; 418:29-37. PMID: 189376440) and altered mitochondrial membrane potential (Chen L B: Mitochondrial membrane potential in living cells, Ann. Rev. Cell Biol. 1988; 4:155-81), relative to normal cells.

In certain embodiments, the TTP agent is 10-TTP or 12-TTP.

In certain embodiments, the TTP agent is a compound of formula I:

Ph₃P⁺-L-W Y⁻ I
wherein:
   W is selected from:

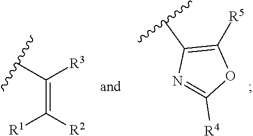

L is absent, (C₁-C₁₂)alkyl, (C₁-C₁₂)alkylene, —(CH₂CH₂O)ₙM—, —C(═O)NR^{L1}—, —NR^{L1}C(═O)— or —NR^{L1}C(═S)NR^{L1}—;
n is 1 to 12;
M is absent or —CH₂CH₂—;
R^{L1} is H or (C₁-C₆)alkyl;
R¹ is halo or —NHC(═O)Rₐ;
R² is halo, SR_{b} or —C(═O)NHR_{c};
R³ is —NH(C═O)R_{d}, —NH(C═O)NHR_{d} or phenyl wherein any phenyl of R³ is optionally substituted with one or more halo, (C₁-C₃)alkyl, (C₁-C₃)haloalkyl, O(C₁-C₃)haloalkyl or —O(C₁-C₃)alkyl;
R⁴ is (C₁-C₆)alkyl or phenyl wherein any phenyl of R⁴ is optionally substituted with one or more halo, (C₁-C₃)alkyl, (C₁-C₃)haloalkyl or —O(C₁-C₃)alkyl;
R⁵ is —S(C₁-C₆)alkyl or —N((C₁-C₆)alkyl)₂;
Rₐ is phenyl optionally substituted with one or more halo, (C₁-C₃)alkyl, (C₁-C₃)haloalkyl or —O(C₁-C₃)alkyl;
R_{b} is phenyl optionally substituted with one or more halo, (C₁-C₃)alkyl, (C₁-C₃)haloalkyl or —O(C₁-C₃)alkyl;
R_{c} is phenyl optionally substituted with one or more halo, (C₁-C₃)alkyl, (C₁-C₃)haloalkyl or —O(C₁-C₃)alkyl;
R_{d} is phenyl optionally substituted with one or more halo, (C₁-C₃)alkyl, (C₁-C₃)haloalkyl or —O(C₁-C₃)alkyl; and
Y is a counterion;
or a pharmaceutically acceptable salt thereof, and a pharmaceutically acceptable diluent or carrier.

In certain embodiments, the anti-cancer agent is ipilimumab.

The term “anti-cancer agent” includes BUPHENYL® (sodium phenylbutyrate, PBA). PBA is formulated as tablets for oral administration and as a powder for oral, nasogastric, or gastrostomy tube administration contain sodium phenylbutyrate. Sodium phenylbutyrate is an off-white crystalline substance which is soluble in water and has a strong salty taste. Sodium phenylbutyrate also is freely soluble in methanol and practically insoluble in acetone and diethyl ether. It is known chemically as 4-phenylbutyric acid, sodium salt with a molecular weight of 186 and the molecular formula C10H₁₁O₂Na.

PBA has the following structure:

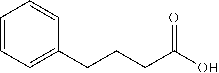 [SMILES: O=C(O)CCCc1ccccc1]

In certain embodiments, Phenylbutyrate is Buphenyl® (sodium phenylbutyrate). Sodium phenylbutyrate is used for chronic management of urea cycle disorders (UCDs). Its mechanism of action involves the quick metabolization of sodium phenylbutyrate to phenylacetate. Phenylacetate then conjugates with glutamine (via acetylation) to form phenylacetylglutamine, and phenylacetylglutamine is excreted by the kidneys. It has been observed that sodium phenylbutyrate reduces Endoplasmic Reticulum (ER) stress.

The cellular response to ER stress is neither fully oncogenic nor completely tumor suppressive. It involves complex signaling with many pathways. The relative importance of each pathway varies between cells depending on chronicity of ER stress, and on relative expression of various associated proteins. As solid cancers grow, nutrients and oxygen required exceed capacity of existing vascular bed, which can trigger angiogenesis (development of new blood vessels) to get more oxygen/nutrients to the cancers. Cancers, however, usually become hypoxic and nutrient-depleted, and with the hypoxia leading to impaired generation of ATP. The low ATP levels compromise ER protein folding which leads to ER stress. Thus, unfolded, and/or misfolded proteins are associated with ER stress and cancer cells exist with higher levels of ER stress relative to health cells.

Potential outcomes as a consequence of ER stress include high rates of protein synthesis that would trigger increased expression of autophagy, which is cytoprotective during stress (liberates amino acids, and removes damaged organelles). Another outcome would be an increased tolerance to hypoxia, which would promote tumor growth. This would also increase autophagy, promoting drug resistance. Thus, a successful treatment would inhibit autophagy and promote cell death.

Sodium phenylbutyrate decreases ER Stress. Lowering ER stress prevents tolerance to hypoxia, and prevents cytoprotective autophagy (which leads to drug resistance). Phenylbutyrate acts as a “chemical chaperone,” meaning it guides proper protein folding, and the presence of properly folded proteins lowers ER stress.

PBA and other histone deacetylase inhibitors (e.g., Vorinastat) upregulate MCR1 expression in metastatic melanoma cells. PBA has a second mechanism of action for the present combination therapy in that it disrupts ER-stress mediated autophagy, which is an underlying mechanism of metastatic melanoma resistance to vemurafenib and MAPK pathway inhibitor treatments. Thus, PBA sensitizes BRAF inhibitor resistant melanoma cells to BRAF inhibition treatment.

### Anti-Cancer Agents

As used herein, the term “anti-cancer agent” includes therapeutic agents that kill cancer cells; slow tumor growth and cancer cell proliferation; and ameliorate or prevent one or more of the symptoms of cancer. An anti-cancer agent includes pharmaceutically acceptable salts. The term “pharmaceutically acceptable salts” refers to salts that retain the desired biological activity of the above-identified compounds, and include pharmaceutically acceptable acid addition salts and base addition salts. Suitable pharmaceutically acceptable acid addition salts may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

In certain embodiments, the anti-cancer agent is a MAPK pathway inhibitor, including but not limited to cobimetinib, dabrafenib, and/or trametinib.

### Immune Checkpoint Inhibitors

As used herein, the term “immune checkpoint inhibitors” include therapeutic agents that block proteins that stop the immune system from attacking cancer cells. An immune checkpoint inhibitor includes pharmaceutically acceptable salts. The term “pharmaceutically acceptable salts” refers to salts that retain the desired biological activity of the above-identified compounds, and include pharmaceutically acceptable acid addition salts and base addition salts. Suitable pharmaceutically acceptable acid addition salts may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

In certain embodiments, the immune checkpoint inhibitor is a CTLA-4, PD-1 inhibitor, and/or a PD-L1 inhibitor including, but not limited to, pembrolizumab (Keytruda®), ipilimumab (Yervoy®), nivolumab (Opdivo®), and/or atezolizumab (Tecentriq®).

### Skin Abnormalities and Cancers

In certain embodiments, the skin abnormality, disease and/or condition includes, but is not limited to, hyperpigmentation (including melasma), hypopigmentation (including vitiligo), melanoma, metastatic melanoma, basal cell carcinoma, squamous cell carcinoma, erythropoietic protoporphyria, polymorphous light eruption, solar urticaria, photosensitivity, sunburn, inflammatory diseases, aberrant fibroblast activity and pain.

In certain embodiments, the skin abnormality is a skin cancer. In certain embodiments, the skin cancer is melanoma. In certain embodiments, the melanoma is metastatic melanoma. In certain embodiments, the melanoma is drug-resistant (e.g., vemurafenib-resistant) metastatic melanoma.

Agents that Increase Expression of MCR1

Vemurafenib (Zelboraf®) is a B-Raf enzyme inhibitor developed for the treatment of late-stage melanoma. Vemurafenib stops the proliferative effects of oncogenic BRAF protein. The name “vemurafenib” comes from V600E mutated BRAF inhibition.

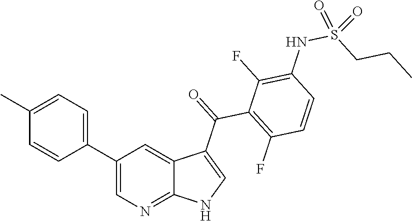 [SMILES: CCCS(=O)(=O)Nc1ccc(F)c(C(=O)c2c[nH]c3ncc(-c4ccc(C)cc4)cc23)c1F]

ZELBORAF® Vemurafenib is indicated for the treatment of patients with unresectable or metastatic melanoma with BRAF V600E mutation as detected by an FDA-approved test. Tumor specimens are confirmed for the presence of BRAF V600E mutation prior to initiation of treatment with Vemurafenib. The recommended dose is 960 mg orally twice daily taken approximately 12 hours apart with or without a meal; 720 mg twice daily for first appearance of intolerable Grade 2 or Grade 3 adverse reactions; or 480 mg twice daily for second appearance of Grade 2 (if intolerable) or Grade 3 adverse reactions or for first appearance of Grade 4 adverse reaction (if clinically appropriate). Unfortunately, metastatic melanoma can resist vemurafenib treatment. Vemurafenib slows tumor progression for only about 5.3 months. As a result, finding an effective treatment for metastatic melanoma is challenging.

The term “anti-cancer agent” includes a Triphenylphosphonium (TPP) agent or derivative thereof that increases reactive oxygen species (ROS) levels in cancer cell mitochondria, and a pharmaceutically acceptable diluent or carrier. As used herein, the term triphenylphosphonium (TPP) is any molecule containing a triphenylphosphine cation (⁺PPh₃) moiety. See, e.g., WO 2013/019975 and WO 2014/124384, which are incorporated by reference herein.

TPP salts can be reacted with alcohols, alkyl halides, and carboxylic acids, which allow them to be used as starting materials for the synthesis of a large variety of chemical derivatives, e.g., XTPP agents. Charged molecules generally cannot pass through cell membranes without the assistance of transporter proteins because of the large activation energies need to remove of associated water molecules. In the TPP molecules, however, the charge is distributed across the large lipophilic portion of the phosphonium ion, which significantly lowers this energy requirement, and allows the TPP to pass through lipid membranes. The phosphonium salts accumulate in mitochondria due to the relatively highly negative potential inside the mitochondrial matrix. The compositions of the present invention utilize XTPP agents that have activity in treating cancer cells, in that the XTPP agents preferentially localize to cancer cells, as compared to the comparable normal cells because cancer cells are often characterized by abnormal mitochondrial oxidative metabolism (Aykin-Burns N, Ahmad I M, Zhu Y, Oberley L W, and Spitz D R: Increased levels of superoxide and hydrogen peroxide mediate the differential susceptibility of cancer cells vs. normal cells to glucose deprivation. Biochem. J. 2009; 418:29-37. PMID: 189376440) and altered mitochondrial membrane potential (Chen L B: Mitochondrial membrane potential in living cells, Ann. Rev. Cell Biol. 1988; 4:155-81), relative to normal cells.

In certain embodiments, the TTP agent is 10-TTP or 12-TTP.

In certain embodiments, the TTP agent is a compound of formula I:

Ph₃P⁺-L-W Y⁻ I
wherein:
   W is selected from:

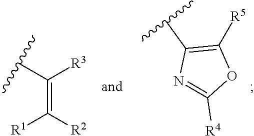

L is absent, (C₁-C₁₂)alkyl, (C₁-C₁₂)alkylene, —(CH₂CH₂O)ₙM—, —C(═O)NR^{L1}—, —NR^{L1}C═O)— or —NR^{L1}C(═S)NR^{L1}—;
n is 1 to 12;
M is absent or —CH₂CH₂—;
R^{L1} is H or (C₁-C₆)alkyl;
R¹ is halo or —NHC(═O)Rₐ;
R² is halo, SR_{b} or —C(═O)NHR_{c};
R³ is —NH(C═O)R_{d}, —NH(C═O)NHR_{d} or phenyl wherein any phenyl of R³ is optionally substituted with one or more halo, (C₁-C₃)alkyl, (C₁-C₃)haloalkyl, O(C₁-C₃)haloalkyl or —O(C₁-C₃)alkyl;
R⁴ is (C₁-C₆)alkyl or phenyl wherein any phenyl of R⁴ is optionally substituted with one or more halo, (C₁-C₃)alkyl, (C₁-C₃)haloalkyl or —O(C₁-C₃)alkyl;
R⁵ is —S(C₁-C₆)alkyl or —N((C₁-C₆)alkyl)₂;
Rₐ is phenyl optionally substituted with one or more halo, (C₁-C₃)alkyl, (C₁-C₃)haloalkyl or —O(C₁-C₃)alkyl;
R_{b} is phenyl optionally substituted with one or more halo, (C₁-C₃)alkyl, (C₁-C₃)haloalkyl or —O(C₁-C₃)alkyl;
R_{c} is phenyl optionally substituted with one or more halo, (C₁-C₃)alkyl, (C₁-C₃)haloalkyl or —O(C₁-C₃)alkyl;
R_{d} is phenyl optionally substituted with one or more halo, (C₁-C₃)alkyl, (C₁-C₃)haloalkyl or —O(C₁-C₃)alkyl; and
Y is a counterion;
or a pharmaceutically acceptable salt thereof, and a pharmaceutically acceptable diluent or carrier.

In certain embodiments, the anti-cancer agent is ipilimumab.

The term “anti-cancer agent” includes BUPHENYL® (sodium phenylbutyrate, PBA). PBA is formulated as tablets for oral administration and as a powder for oral, nasogastric, or gastrostomy tube administration contain sodium phenylbutyrate. Sodium phenylbutyrate is an off-white crystalline substance which is soluble in water and has a strong salty taste. Sodium phenylbutyrate also is freely soluble in methanol and practically insoluble in acetone and diethyl ether. It is known chemically as 4-phenylbutyric acid, sodium salt with a molecular weight of 186 and the molecular formula C10H₁₁O₂Na.

PBA has the following structure:

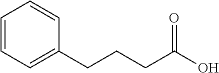 [SMILES: O=C(O)CCCc1ccccc1]

In certain embodiments, Phenylbutyrate is Buphenyl® (sodium phenylbutyrate). Sodium phenylbutyrate is used for chronic management of urea cycle disorders (UCDs). Its mechanism of action involves the quick metabolization of sodium phenylbutyrate to phenylacetate. Phenylacetate then conjugates with glutamine (via acetylation) to form phenylacetylglutamine, and phenylacetylglutamine is excreted by the kidneys. It has been observed that sodium phenylbutyrate reduces Endoplasmic Reticulum (ER) stress.

The cellular response to ER stress is neither fully oncogenic nor completely tumor suppressive. It involves complex signaling with many pathways. The relative importance of each pathway varies between cells depending on chronicity of ER stress, and on relative expression of various associated proteins. As solid cancers grow, nutrients and oxygen required exceed capacity of existing vascular bed, which can trigger angiogenesis (development of new blood vessels) to get more oxygen/nutrients to the cancers. Cancers, however, usually become hypoxic and nutrient-depleted, and with the hypoxia leading to impaired generation of ATP. The low ATP levels compromise ER protein folding which leads to ER stress. Thus, unfolded, and/or misfolded proteins are associated with ER stress and cancer cells exist with higher levels of ER stress relative to health cells.

Potential outcomes as a consequence of ER stress include high rates of protein synthesis that would trigger increased expression of autophagy, which is cytoprotective during stress (liberates amino acids, and removes damaged organelles). Another outcome would be an increased tolerance to hypoxia, which would promote tumor growth. This would also increase autophagy, promoting drug resistance. Thus, a successful treatment would inhibit autophagy and promote cell death.

Sodium phenylbutyrate decreases ER Stress. Lowering ER stress prevents tolerance to hypoxia, and prevents cytoprotective autophagy (which leads to drug resistance). Phenylbutyrate acts as a “chemical chaperone,” meaning it guides proper protein folding, and the presence of properly folded proteins lowers ER stress.

PBA and other histone deacetylase inhibitors (e.g., Vorinastat) upregulate MCR1 expression in metastatic melanoma cells. PBA has a second mechanism of action for the present combination therapy in that it disrupts ER-stress mediated autophagy, which is an underlying mechanism of metastatic melanoma resistance to vemurafenib and MAPK pathway inhibitor treatments. Thus, PBA sensitizes BRAF inhibitor resistant melanoma cells to BRAF inhibition treatment.

### Compositions and Methods of Administration

The present invention provides a method for increasing the anticancer effects of a conventional cancer therapy (i.e., radio- and/or chemo-therapy) on cancerous cells in a mammal, comprising contacting the cancerous cell with an effective amount of a melanoma-targeting conjugate comprising Formula I:

T-L-X
wherein T is a MCR1 Ligand,
L is a linker, and
X an anti-cancer composition,
for the therapeutic treatment of melanoma.

In certain embodiments, the conjugate is administered along with an additional conventional cancer therapy modality. In certain embodiments, the additional cancer therapy is chemotherapy and/or radiation. In certain embodiments, the conjugate of Formula I and anti-cancer agent are administered sequentially to a mammal rather than in a single composition. In certain embodiments, the mammal is a human.

The present invention provides a method for increasing the anticancer effects of a conventional cancer therapy (i.e., radio- and/or chemo-therapy) on cancerous cells in a mammal, comprising contacting the cancerous cell with an effective amount of an agent that increases expression of MCR1 and with an MCR1 ligand.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The present invention provides a “substantially pure compound”. The term “substantially pure compound” is used herein to describe a molecule, such as a polypeptide (e.g., a polypeptide that binds MC1R, or a fragment thereof) that is substantially free of other proteins, lipids, carbohydrates, nucleic acids, and other biological materials with which it is naturally associated. For example, a substantially pure molecule, such as a polypeptide, can be at least 60%, by dry weight, the molecule of interest. The purity of the polypeptides can be determined using standard methods including, e.g., polyacrylamide gel electrophoresis (e.g., SDS-PAGE), column chromatography (e.g., high performance liquid chromatography (HPLC)), and amino-terminal amino acid sequence analysis.

“Treatment”, “treating”, “treat” or “therapy” as used herein refers to administering, to a mammal, agents that are capable of eliciting a prophylactic, curative or other beneficial effect in the individual. Treatment may additionally result in attenuating or ameliorating a disease or symptoms of a disease in a subject.

In certain embodiments, the conjugate is administered along with an additional conventional cancer therapy modality. In certain embodiments, the additional cancer therapy is chemotherapy and/or radiation. In certain embodiments, the agent that increases expression of MCR1 and an MCR1 ligand are administered sequentially to a mammal rather than in a single composition. In certain embodiments, the mammal is a human.

In certain embodiments of the methods described above, agent that increases expression of MCR1 does not significantly inhibit viability of comparable non-cancerous cells.

In certain embodiments of the methods described above, the tumor is reduced in volume by at least 10%. In certain embodiments, the tumor is reduced by any amount between 1-100%. In certain embodiments, the tumor uptake of molecular imaging agents, such as fluorine-18 deoxyglucose, fluorine-18 thymidine or other suitable molecular imaging agent, is reduced by any amount between 1-100%. In certain embodiments the imaging agent is fluorine-18 deoxyglucose, fluorine-18 thymidine or other suitable molecular imaging agent. In certain embodiments, the mammal's symptoms (such as flushing, nausea, fever, or other maladies associated with cancerous disease) are alleviated.

Administration of a compound as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The agent that increases expression of MCR1 and the MCR1 ligand can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it may be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The dosage of the agent that increases expression of MCR1 and the MCR1 ligand varies depending on age, weight, and condition of the subject. Treatment may be initiated with small dosages containing less than optimal doses, and increased until a desired, or even an optimal effect under the circumstances, is reached. In general, the dosage is about 450-600 mg/kg/day in patients weighing less than 20 kg, or 9.9-13.0 g/m²/day in larger patients. Higher or lower doses, however, are also contemplated and are, therefore, within the confines of this invention. A medical practitioner may prescribe a small dose and observe the effect on the subject's symptoms. Thereafter, he/she may increase the dose if suitable. In general, agent that increases expression of MCR1 and the MCR1 ligand are administered at a concentration that affords effective results without causing any unduly harmful or deleterious side effects, and may be administered either as a single unit dose, or if desired in convenient subunits administered at suitable times.

The dosage of the immune checkpoint inhibitor will depend upon the ICI(s) used in the treatment. However, in general, the ICI(s) may be administered at any concentration(s) that afford effective results without causing any unduly harmful or deleterious side effects and may be administered either as a single unit dose, or if desired in convenient subunits administered at suitable times. Typical dosages include, but are not limited to:
atezolizumab (Tecentriq): 600 mg-1800 mg IV every week to 4 weeks;
ipilimumab (Yervoy): 1.0-5.0 mg/kg IV every 2-4 weeks;
nivolumab (Opdivo): 0.5-5.0 mg/kg IV every 1-3 weeks;
pembrolizumab (Keytruda): 50-600 mg IV every 4-8 weeks.

A pharmaceutical composition of the invention is formulated to be compatible with its intended route of administration. For example, the therapeutic agent may be introduced directly into the cancer of interest via direct injection. Additionally, examples of routes of administration include oral, parenteral, e.g., intravenous, slow infusion, intradermal, subcutaneous, oral (e.g., ingestion or inhalation), transdermal (topical), transmucosal, and rectal administration. Such compositions typically comprise the agent that increases expression of MCR1 and the MCR1 ligand and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration, and a dietary food-based form. The use of such media and agents for pharmaceutically active substances is well known in the art and food as a vehicle for administration is well known in the art.

Solutions or suspensions can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampules, disposable syringes, or multiple dose vials made of glass or plastic.

It may be advantageous to formulate compositions in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the invention are dependent upon the amount of a compound necessary to produce the desired effect(s). The amount of a compound necessary can be formulated in a single dose, or can be formulated in multiple dosage units. Treatment may require a one-time dose, or may require repeated doses.

“Systemic delivery,” as used herein, refers to delivery of an agent or composition that leads to a broad biodistribution of an active agent within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent directly to a target site within an organism. For example, an agent can be locally delivered by direct injection into a disease site, other target site, or a target organ such as the skin.

The term “mammal” refers to any mammalian species such as a human, mouse, rat, dog, cat, hamster, guinea pig, rabbit, livestock, and the like.

The terms “treat” and “treatment” refer to both therapeutic treatment and prophylactic or preventative measures, wherein the object is to prevent or decrease an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The invention will now be illustrated by the following non-limiting Examples.

### Example 1

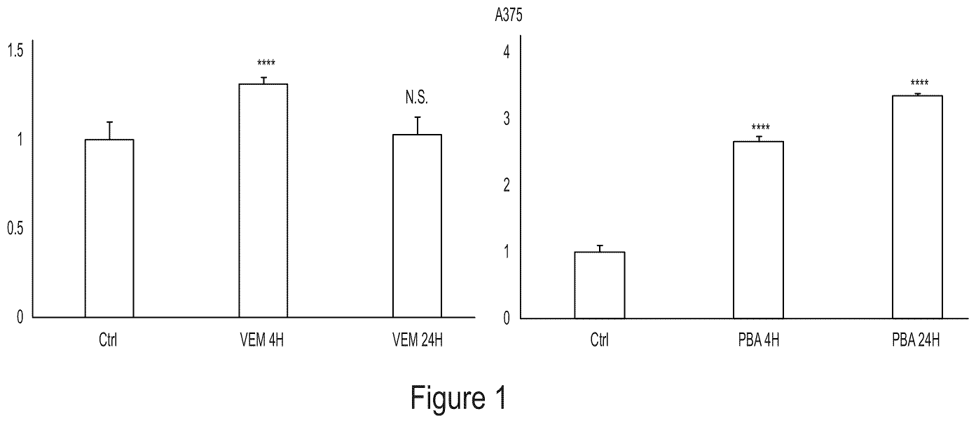

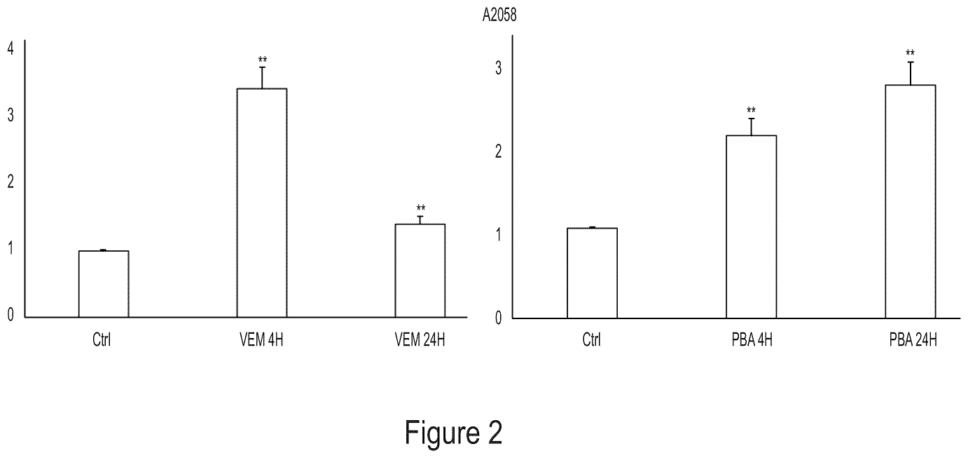

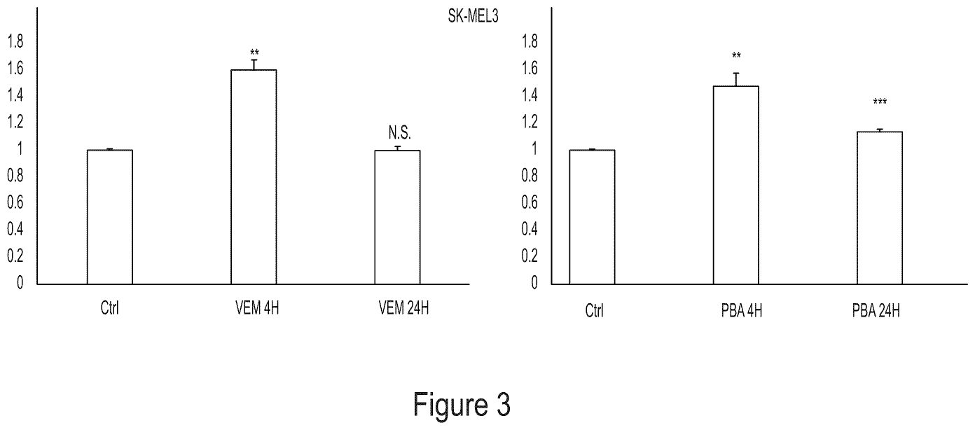

Experiments were performed showing that it is possible to up-regulate the MCR1 receptor expression pharmacologically (FIGS. 1-3). Cells were treated with clinically relevant concentrations of agents and analyzed by flow cytometry for expression of the MCR1 protein.

### Example 2

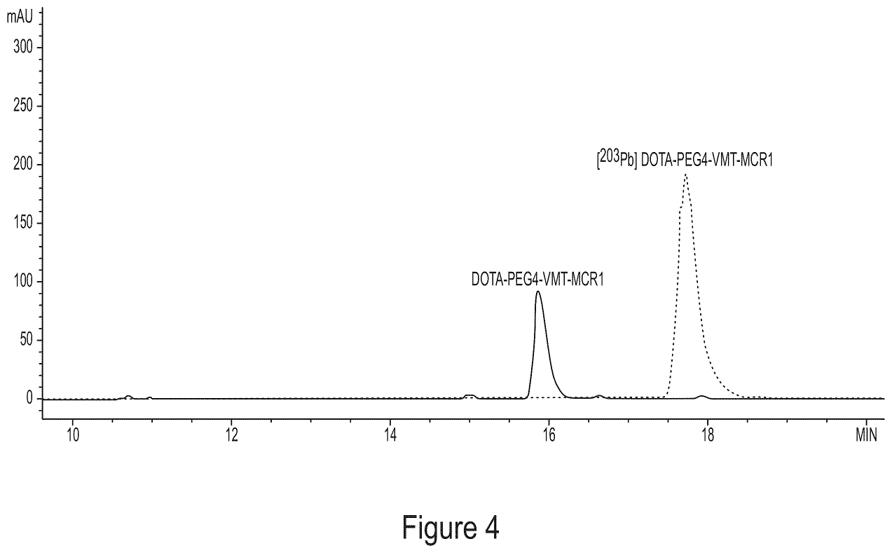

Experiments were performed showing that the internalization of linker modified variants were superior to conjugates lacking linkers. A typical radio-HPLC chromatogram of co-injection of DOTA-PEG4-VMT-MCR1 and [²⁰³Pb]DOTA-PEG4-VMT-MCR1 is shown in FIG. 4. [²⁰³Pb]DOTA-PEG4-VMT-MCR1 was radiolabeled by standard Pb-resin based method. An aliquot of 37kBq [²⁰³Pb]DOTA-PEG4-VMT-MCR1 was mixed with 10 μg of DOTA-PEG4-VMT-MCR1 before injection. The retention time of both content were monitored by UV signal at 280 nm and β-RAM radio-detector; Gradient: linear 16%-26% of acetonitrile in 20 mM HCl over 20 minutes on Vyldac 218TP C18 column (4.6×150 mm 5 μm) with 1 ml/min flow rate.

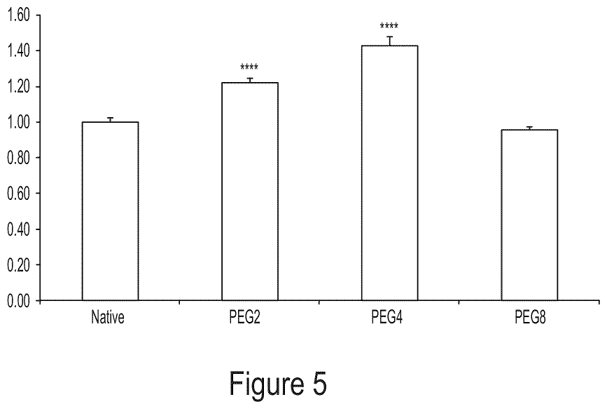

FIG. 5 provides data from a two hour internalization study of linker modified variants. 200,000 count per minute (CPM) HPLC purified [Pb-203]DOTA-linker-VMT-MCR1 was added to 0.2 million B16 mice melanoma cells in 24-well plate. After 2 h incubation under room temperature, media was removed. Cells were harvested and counted by NaI gamma detector. Data are expressed as internalization relative to original no-linker peptide±SEM. Significance is expressed as P<0.05*, P<0.01**, P<0.001***, P<0.0001****. These results demonstrate a surprising, yet significant improvement in cellular internalization when the DOTA-VMT-MCR1 peptide is modified to include a PEG4 linker between the chelator and the peptide that reduces steric hindrances of peptide binding to the MCR1.

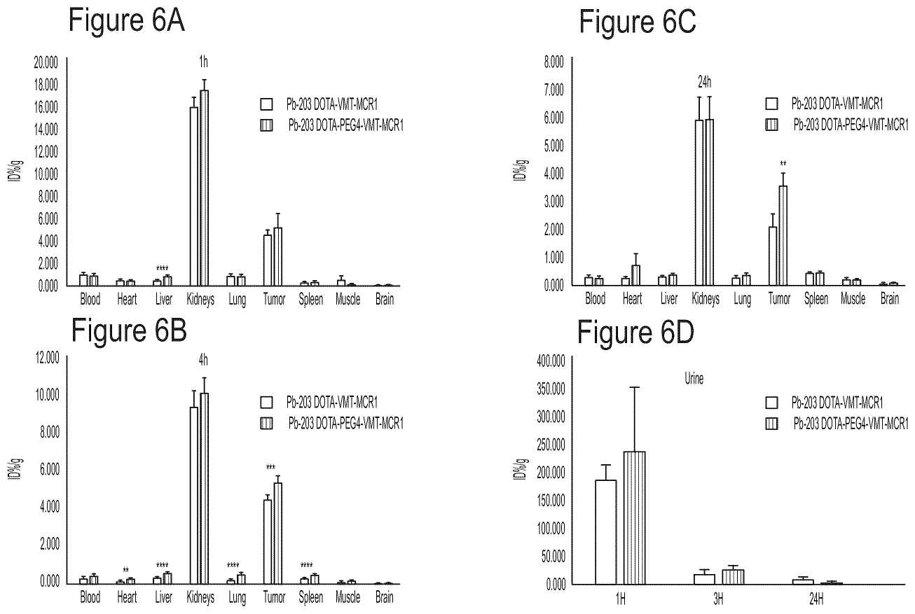

FIGS. 6A-6D provide the pharmacokinetics characteristics of [²⁰³Pb]DOTA-VMT-MCR1 and [²⁰³Pb]DOTA-PEG4-VMT-MCR1 in B16/F1 murine melanoma-bearing C₅₇ mice. 0.037MBq of each compound was injected via tail vein. Mice were euthanized at (FIG. 6A) 1 h, (FIG. 6B) 4 h and (FIG. 6C) 24 h (n=3). (FIG. 6D) provides data for urine samples collected ad 1 h, 3 h and 24 h. Tumor and organs of interest were harvested. Radioactivity was measured by NaI gamma detector. Results were expressed as percent injected dose per gram of tissue (ID %/g)±SEM. P<0.05*, P<0.01**, P<0.001***, P<0.0001****. These results demonstrate a surprising, yet significant improvement in tumor accumulation and retention when the DOTA-VMT-MCR1 peptide is modified to include a PEG4 linker between the chelator and the peptide that reduces steric hindrances of peptide binding to the MCR1.

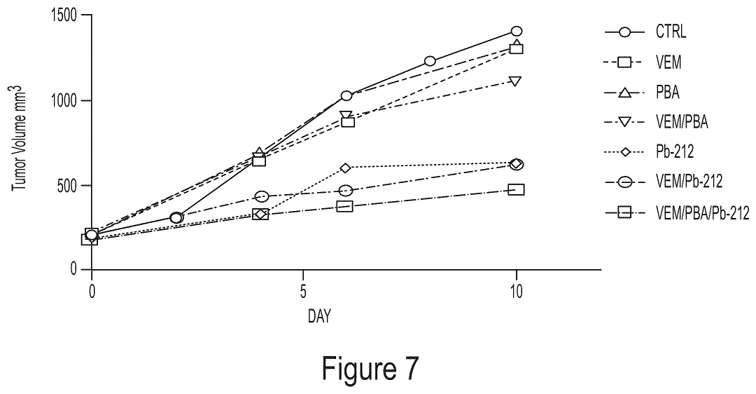

FIG. 7. [Pb-212]DOTA-PEG4-VMT-MCR1 improved therapy for metastatic melanoma tumors in mice compared to standard of care BRAFᵢ. Mice bearing A2058 tumor xenografts were administered with vehicle (CTRL); 10 mg/kg Vemurafenib (BRAFᵢ) twice a day (VEM); i.p. injected 60 mg/kg 4-PBA (PBA); i.v. injected 120 μCi of [²¹²Pb]DOTA-VMT-MCR1 in 3 fractions over 6 days (PB-212); or the combinations (VEM/PBA, VEM/PB-212 and VEM/PBA/VEM 212). Average tumor volumes with SDs were determined from 9-10 animals per group. Experiments conducted according to animal protocols approved by the University of Iowa Animal Care and Use Committee (IACUC).

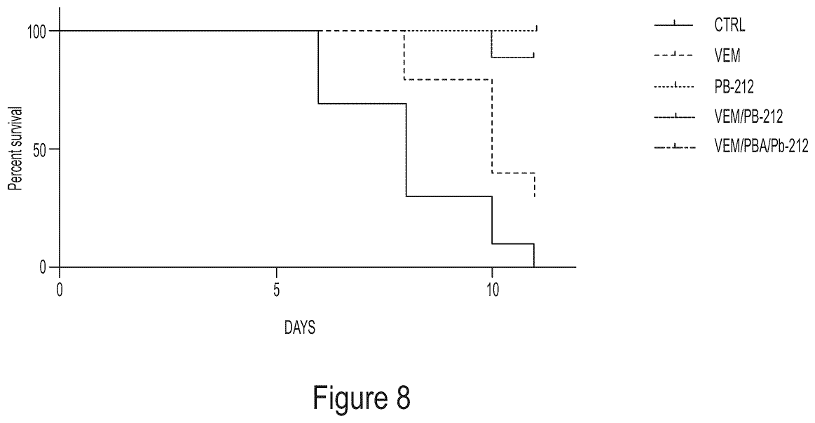

FIG. 8. [Pb-212]DOTA-PEG4-VMT-MCR1 therapy for metastatic melanoma tumors in mice improved survival compared to standard of care BRAFᵢ. Mice bearing A2058 human melanoma tumor xenografts were administered with vehicle (CTRL); 10 mg/kg Vemurafenib (BRAFi) twice a day (VEM); i.p. injected 60 mg/kg 4-phenylbutyrate (PBA); i.v. injected 120 μCi of [²¹²Pb]DOTA-VMT-MCR1 in 3 fractions over 6 days (PB-212); or the combinations (VEM/PBA, VEM/PB-212 and VEM/PBA/VEM 212). Animal were euthanized when tumor size reached 1500 mm³, loss of 30% body weight; or in case of ruptured tumor ulceration. Experiments conducted according to animal protocols approved by the University of Iowa Animal Care and Use Committee (IACUC).

FIG. 9. Examples of linkers: (1) polyethyleneglycol (PEG)-based linkers with 2, 4, and 8 PEG subunits; (2) aliphatic (ALP) linkers of 2 and 4 carbons; and a piperidine (PIP) based linker with mixed characteristics.

These experiments show a significant improvement to the internalization of the conjugate on binding to the MCR1 protein on melanoma cells, which allows for significantly enhancing tumor retention and ration dose to the tumor relative to other organs and tissues. The conjugate is internalized and retained better in melanoma cells than the previous DOTA-VMT-MCR1 molecules for this purpose. Also, surprisingly, it is taken up and retained significantly better in melanoma tumors than previous molecules for this purpose (FIG. 6). Moreover, it was found that the conjugate was very surprisingly, much easier to purify the radiolabeled version (which is used of imaging ant therapy) than others previously (FIG. 4), making it much more useful for radiopharmaceutical use for clinical use for therapy and imaging of patients.

### Example 3

Melanoma if detected early can be cured by surgery, but metastatic melanoma is lethal. New therapies (e.g., immunotherapy; BRAF inhibition, BRAFᵢ) and combinations extend life, but low response rates, acquired drug resistance, and serious side effects are major challenges to improving outcomes for disseminated disease (5 y survival 17%). Peptide-based radionuclide therapy targeted to the melanocortin-receptor type 1 (MCR1-RT) has long been considered a promising alternative; and MCR1-RT has achieved complete responses in mouse models (B16). However, MCR1 expression is heterogeneous/low in human melanoma, and as a result, no previous MCR1-RT study has employed successfully human melanoma cell xenografts. MCR1 expression can be significantly enhanced pharmacologically in human melanoma cells via incubation with FDA-approved drugs including Buphenyl™ (4-phenylbutyrate; PBA; up to 8-fold); MAPK-targeted melanoma drugs (BRAFᵢ and MEKᵢ); and histone deacetylase inhibitor (HDACᵢ) Vorinostat (up to 12-fold). In vivo, combining [²¹²Pb]DOTA-MCR1 α-therapy with PBA and BRAFᵢ significantly improved tumor response and survival of mice bearing human melanoma tumor xenografts (A2058, 451-LU, A375) compared to BRAFᵢ or [²¹²Pb]DOTA-MCR1 alone. [²¹²Pb]DOTA-MCR1 with PBA was also effective in mice bearing human BRAF^{WT} (MeWo) tumors. Furthermore, co-injection of PBA with [²⁰³Pb]DOTA-MCR1 significantly reduced radiopeptide accumulation in kidney (dose limiting organ); and PBA combined with BRAFᵢ promoted cell death of BRAFᵢ-resistance melanoma cells, suggesting additional roles involving ER stress for PBA. This introduces innovative Pb-specific chelator combined with a “click-cyclized” peptide (PSC-C-MCR1) to improve tumor:kidney ratio of radionuclide uptake 7-fold. An effective therapy for metastatic melanoma is developed that combines MCR1-RT with pharmacological agents (PBA; BRAFᵢ/MEKᵢ; HDACᵢ) that enhance MCR1 expression.

Melanoma is the fastest growing cancer incidence in the United States. Surgery combined with radiation can be curative at early stages. However, metastatic melanoma is almost uniformly fatal (5-yr survival 17%). Recent breakthrough targeted MAPKᵢ therapies (e.g., BRAFᵢ) and immunotherapies (e.g., PD-1 inhibitors) have improved outcomes, but low response rates, acquired drug resistance, and adverse side effects limit quality of life for metastatic melanoma patients. For example, approval of immune-checkpoint inhibitor ipilimumab was based on an improvement in overall survival of only 3.7 months (overall response <15%). Combination immunotherapies have improved response (up to 61%), but grade 3/4 adverse events (up to 55%) often lead to therapy discontinuation (up to 36%). For targeted therapies, BRAF inhibitor vemurafenib was approved based on overall survival at 6 months of 84% vs 64% in the control arm (dacarbazine). Combining BRAFᵢ with MEK inhibitors (MEKᵢ) has led to modest improvements, yet recurrence is virtually inevitable. The mechanisms of acquired drug resistance are complex, and include altered/alternative oncogenic pathways; tumor heterogeneity; and enhanced DNA repair. Melanocortin-receptor type 1 targeted radionuclide therapy (MCR1-RT) has long been considered a promising alternative treatment for melanoma; and MCR1-targeted α-particle therapy (²¹²Pb) has achieved complete responses in mice bearing mouse (B16) tumors that highly express MCR1. However, these studies have been confined primarily to mouse melanoma (B16) cells because MCR1 expression is heterogeneous/low in human melanoma. On the other hand, data reveal that MCR1 expression can be significantly enhanced pharmacologically in human melanoma cells via treatment with FDA-approved drug Buphenyl™ (4-phenylbutyrate; PBA; up to 8-fold). Further, incubation with FDA-approved melanoma drugs (MAPK pathway inhibitors BRAFᵢ and MEKᵢ) and histone deacetylase inhibitor (HDACᵢ) Vorinostat also significantly enhanced MCR1 expression (up to 12-fold). In vivo, the combination of [²¹²Pb]DOTA-MCR1 α-particle therapy with PBA and BRAFᵢ significantly improved tumor response and survival of mice bearing human melanoma xenografts (A2058, 451-LUBR, A375) tumors compared to BRAFᵢ or [²¹²Pb]DOTA-MCR1 alone. PBA combined with [²¹²Pb]DOTA-MCR1 was also effective in mice bearing human BRAF^{WT} (MeWo) tumors. Furthermore, co-injection of PBA with [²⁰³Pb]DOTA-MCR1 significantly reduced radiopeptide accumulation in kidney; and PBA combined with BRAFᵢ promoted cell death of BRAFᵢ-resistance melanoma cells, suggesting additional roles for PBA.

MCR1-targeted radionuclide therapy has been long considered promising; and numerous α-MSH analogs that bind MCR1 using mouse B16 (F1/F10) melanoma cells that highly express the MCR1 target. However, MCR1-targeted radionuclide therapy using human melanoma cells has not been reported previously. Thus, the present experiments are novel because it is shown that MCR1 expression in human melanoma cells can be robustly enhanced pharmacologically (in vitro and in vivo) using FDA-approved melanoma BRAFᵢ/MEKᵢ drugs, Buphenyl (PBA) and HDACᵢ vorinostat. It is important to note that the present data show that this innovation produces robust MCR1 expression in human melanoma tumor xenografts in mice, and that [²¹²Pb]DOTA-MCR1+BRAFᵢ+PBA significantly improved survival of human melanoma tumor bearing mice and significantly reduced tumor growth rates relative to standard of care BRAFᵢ and [²¹²Pb]DOTA-MCR1 alone. In addition, data further resulted in the present design, synthesis, and evaluation of a new Pb-specific chelator and polyethylene-based linker that connects the chelator to the MCR1-peptide backbone that significantly improves radiolabeling efficiency; and also improves internalization of MCR1-radiopeptides.

PBA combined with [²¹²Pb]DOTA-MCR1 was produced robust tumor response and survival in mice bearing human BRAF^{WT} (MeWo) tumors. Furthermore, co-injection of PBA with [²⁰³Pb]DOTA-MCR1 significantly reduced radiopeptide accumulation in kidney; and PBA combined with BRAFᵢ promoted cell death of BRAFᵢ-resistance melanoma cells, suggesting additional roles for PBA. Importantly, PBA is an FDA-approved drug prescribed at high doses (up to 27 g/day) prescribed for patients with urea disorders and has been shown to prevent ER-stress induced fibrosis of proximal tubular cells. Thus, the inclusion of PBA co-injection reduces kidney accumulation of the radiopeptides. Thus, the present invention simultaneously reduces radiation dose to the kidneys; decreases oxidative-stress and ER-stress-mediated kidney fibrosis; promotes cell death of BRAFᵢ resistant melanoma; and enhances tumor-specific radiation dose delivery and cell killing.

The present experiments compare outcomes of MCR1-targeted radionuclide therapy for metastatic melanoma using alpha and beta-emitting radionuclides ¹⁷⁷Lu; ²¹²Pb; ⁹⁰Y in mice bearing human melanoma tumors. Bio-distribution studies are carried out using generator gamma emitter ²⁰³Pb that has a 52 h half-life to extend these studies to longer endpoints. Animal studies are conducted using immune compromised (athymic nu nu and NSG) mice (male/female) to compare directly to previous published studies of targeted radionuclide therapy for melanoma in mice.

Introduction: Combination therapies for metastatic melanoma are emerging as common practice (e.g., BRAFᵢ plus MEKᵢ; and combination immunotherapies), but melanoma almost invariably develops resistance. MCR1-RT has long been considered a promising alternative, but previous therapy studies have been limited to the use of B16 mouse melanoma cells that highly express the MCR1 protein, because native MCR1 expression in human melanoma is heterogeneous/low. Preliminary data was part of an investigation into the acquisition of resistance of BRAF^{V600E} metastatic melanoma, which led to the discovery that incubation of FDA approved BRAFᵢ, MEKᵢ, PBA, and HDACᵢ drugs can be used to significantly enhance the expression of MCR1 (FIG. 10). Enhanced expression of MCR1 in response to BRAFᵢ, MEKᵢ, PBA, and HDACᵢ (Vorinostat, aka SAHA) is observed by RT-PCR (FIG. 10); and substantiated by flow cytometry (FIGS. 11A-11B) and by MCR1-radiopeptide binding assays (FIGS. 11C-11D).

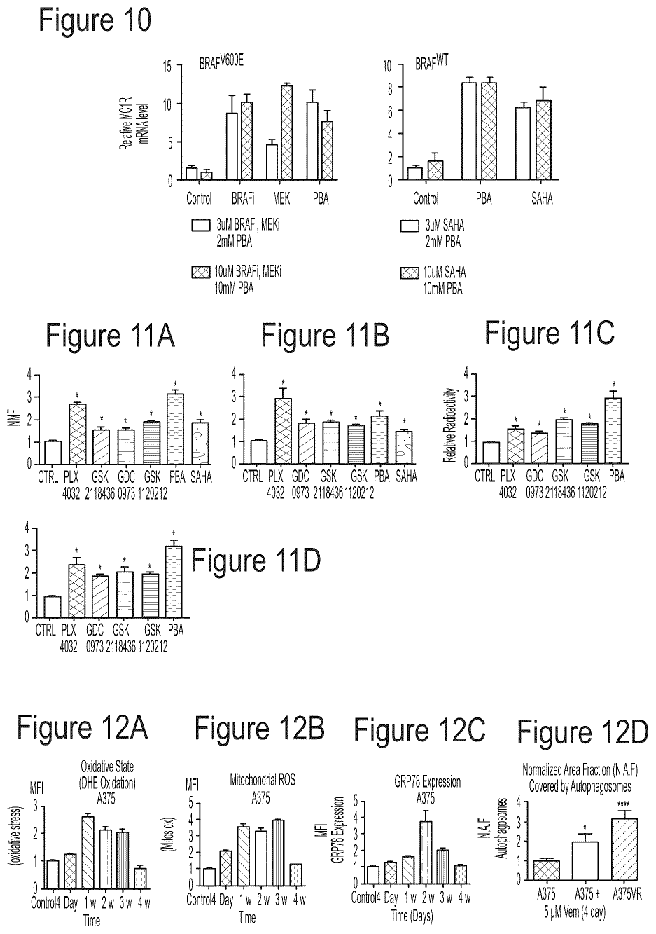

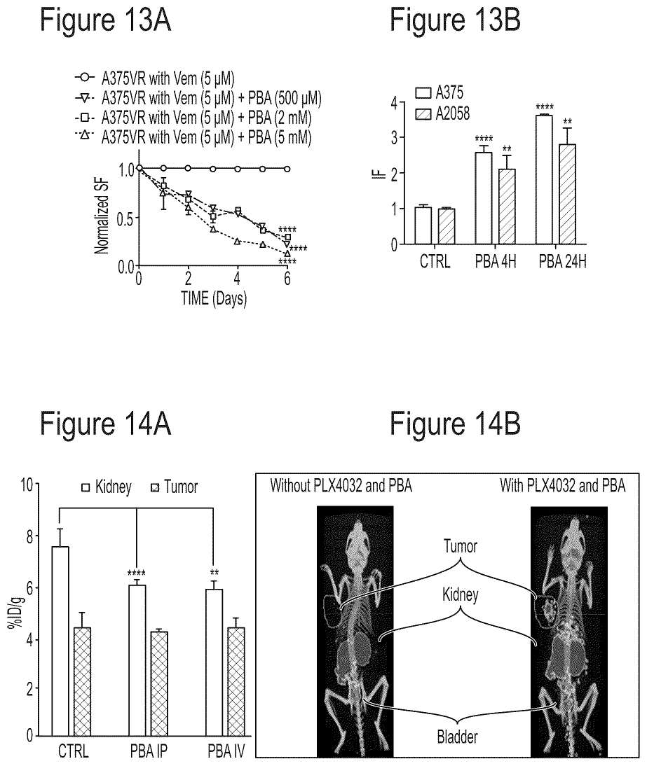

Additional Roles for PBA Involving ER Stress. Sodium 4-phenylbutyrate is a short-chain fatty-acid prodrug that is FDA-approved for patients with urea cycle disorders, and is under investigation for cancer therapy by virtue of HDACᵢ activity. PBA (tradename Buphenyl®) is tolerated in patients at very high doses (up to 27 g/day). The present data suggest additional roles for PBA in the proposed MCR1-RT combination therapy that involves ER stress and acquisition of resistance to BRAFᵢ. Evidence suggests complex mechanisms of acquired drug resistance in metastatic melanoma. It is believed that a primary underlying mechanism of BRAFᵢ-resistance is a metabolic switch that leads to depletion of glutathione levels and a concomitant increase in oxidative state, leading to (ER) stress (evidenced by a significant increase in ER stress marker GRP78; FIG. 12C) that initiates an autophagy response that conveys resistance to BRAFᵢ(FIGS. 12A-12D). These results are important because the data further show that PBA (known as a molecular chaperone that relieves ER stress) promotes cell death of resistant melanoma cells by inhibiting ER-stress mediated autophagy, suggesting an additional role for PBA (FIGS. 13A-13B).

PBA Promotes Cell Death of Melanoma Cells that Have Acquired Resistance to BRAFᵢ Treatment. Accumulation of misfolded-proteins causes upregulation and detachment of ER resident protein GRP78 from ER sensors proteins—activating the unfolded protein response (UPR) and downstream signaling pathways (including autophagy). These observations and initial suggest the relationship between BRAFᵢ; oxidative and ER stresses; autophagy; BRAFᵢ resistance and MCR1 receptor expression. Thus, the potential for a pharmacological treatment that would sensitize BRAFᵢ-resistant cells to BRAFᵢ by reducing ER-stress was considered. BRAFᵢ-resistant A375VR melanoma cells were incubated with BRAFᵢ vemurafenib alone and in combination with ER-stress relieving PBA. Results show that incubating BRAFᵢ-resistant melanoma cells with PBA significantly sensitizes BRAFᵢ-resistant cells to BRAFᵢ, resulting in 90% clonogenic cell death (FIG. 13A). Interestingly, PBA is also known to have histone deacetylase inhibitor activity and histone deacetylase inhibitors have been recognized as pharmacological agents that can drive cell surface expression of GPCRs (e.g., MCR1). A further examination of the time dependency of PBA enhancement of MCR1 in melanoma cell lines revealed a significant (time dependent) increase in MCR1 expression in BRAF^{V600E} mutant melanoma cell lines examined with PBA incubation (FIG. 13B). These results suggest that PBA enhances MCR1 expression through activity as an HDACᵢ, but promotes cell death of resistant melanoma by acting as a chaperone to relieve the ER of misfolded proteins.

PBA Co-Injection Significantly Reduced Kidney Accumulation of [²⁰³Pb]MCR1-Peptide in Mice Bearing Human Melanoma Tumors. PBA relieves ER-stress through non-specific binding to misfolded proteins in the ER. Thus, it was hypothesized that co-injection could inhibit peptide uptake in kidneys in the same fashion as amino acid co-infusions used clinically. This is reasonable because the megalin-cubulin system for reuptake and recirculation of nutrients in the proximal and distal tubules involves nonspecific binding and endocytosis of proteins, amino acids, and nutrients. results support the hypothesis that co-injection of PBA can serve to reduce kidney accumulation of radiopeptides in the kidneys (FIGS. 14A-14B). It is important to note that emerging evidence suggests a protective effect of PBA in kidney tubules, where ER-stress mediated tubular cell apoptosis is increasingly recognized as a mechanism that leads to fibrosis.

Use of PBA Can Improve SPECT Imaging of Human Melanoma Tumors. To further test the idea that PBA can be used to enhance MCR1 expression in human melanoma, SPECT/CT imaging was conducted of mice bearing human melanoma tumors (with and without pre-administration of PBA prior to the injection of a [²⁰³Pb]DOTA-MCR1 peptide). Pre-administration of PBA and BRAFᵢ vemurafenib significantly improved (enabled) SPECT/CT imaging of A2058 BRAF^{V600E} tumor xenograft, while identical imaging settings failed to identify an identical tumor (same size) in an identical mouse (FIG. 14B).

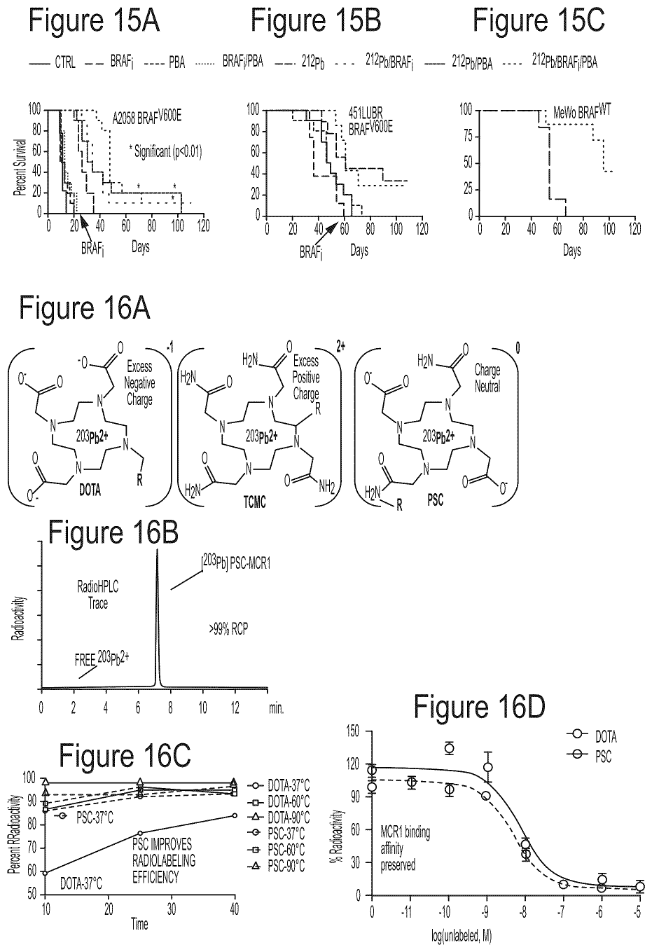

Pharmacologically-Induced MCR1 Expression Using PBA and BRAFᵢ Combined with MCR1-RT (²¹²Pb Alpha Therapy) Improves Tumor Response and Survival of Mice Bearing Human Melanoma Tumors. The in vitro MCR1 receptor expression enhancement and SPECT/CT imaging data support the hypothesis that an effective therapy for metastatic melanoma can be developed that combines MCR1-RT with pharmacological agents (PBA; BRAFᵢ/MEKᵢ; HDACᵢ) that enhance MCR1 expression. Emerging evidence suggests that alpha-particle peptide-receptor-targeted radionuclide therapy may have advantages over beta particle therapy. In addition, alpha-particle MCR1-RT achieved complete responses in nearly 50% of mice bearing B16 mouse tumors in a previous preclinical study. Preliminary in vivo evaluation of the proposed combination MCR1-RT employed three human melanoma xenograft tumor models (FIGS. 15A-15C). For these studies, human mouse tumors were induced subcutaneously and standardized to 100 mm³ prior to the initiation of treatments. Radiopeptides were produced using methods recently published. (Leachman S A, Cassidy P B, Chen S C, Curiel C, Geller A, Gareau D, Pellacani G, Grichnik J M, Malvehy J, North J, Jacques S L, Petrie T, Puig S, Swetter S M, Tofte S, Weinstock M A. Methods of Melanoma Detection. Cancer Treat Res. 2016:167:51-105.) Animals were treated with [²¹²Pb]DOTA-MCR1—with and without BRAFᵢ (vemurafenib), PBA, and combinations shown. Mice were euthanized when tumors reached 1500 mm³ or ulcerations appeared. Significant improvement in survival is observed for BRAF^{V600E} human melanoma tumors in combination with BRAFᵢ and PBA (FIG. 15A). For the 451LUBR tumor model, mice treated with [²¹²Pb]DOTA-MCR1 alone and mice treated with [²¹²Pb]DOTA-MCR1+BRAFᵢ÷PBA showed robust response relative to BRAFᵢ alone (FIG. 15B). BRAFᵢ treatment was not included in the combination with [²¹²Pb]DOTA-MCR1 for MeWo BRAF^{WT} tumor model because BRAFᵢ is not indicated for BRAF^{WT} patients. Nonetheless, the combination MCR1-RT with PBA significantly improved survival in these mice relative to untreated controls (FIG. 15C). These data support the hypothesis that effective therapy for metastatic melanoma can be developed that combines MCR1-RT with pharmacological agents (PBA; BRAFᵢ/MEKᵢ; HDACᵢ) that enhance MCR1 expression.

PSC Chelator: A New Pb-Specific Chelator Improves Radiolabeling efficiency for ²⁰³Pb/²¹²Pb Theranostics. The DOTA chelator has proved useful for gathering preliminary data and provides an efficient platform for trivalent radiometals (⁶⁸Ga, ¹⁷⁷Lu, ⁹⁰Y). However, the most stable oxidation state of Pb is 2+, resulting in a residual −1 charge on the chelate (FIG. 16A). A second chelator (TCMC), has been introduced commercially (FIG. 16A). Researcher concluded that DOTA was a superior chelator for Pb compared to TCMC. (Chappell L L, Dadachova E, Milenic D E, Garmestani K, Wu C, Brechbiel M W. Synthesis, characterization, and evaluation of a novel bifunctional chelating agent for the lead isotopes 203Pb and 212Pb. Nucl Med Biol. 2000; 27(1):93-100.) The authors speculated that TCMC may have low pH stability advantages in lysosomes, although the data presented suggest comparable stability to DOTA at pH 5.5 (the pH of lysosomes). Further, the TCMC-Pb complex results in a net 2⁺ residual charge (FIG. 16A), which has the potential to increase kidney retention through electrostatic interaction with negatively charged surface of tubular cells. Thus, the development of the PSC is based on the chemical principle that minimizing charge (via two carboxy groups) contributes significantly to stability; and that the charge neutral complex does not increase the risk of kidney retention. Data demonstrate that PSC-peptides can be radiolabeled in high radiochemical purity (FIG. 16B); at lower temperatures than DOTA (FIG. 16C); and that PSC does not interfere with receptor binding (FIG. 16D). Thus, PSC is likely to provide the most efficient radiolabeling and stability performance for ²⁰³Pb²⁺/²¹²Pb²⁺ divalent cations. A DOTA-based conjugate of the MCR1-targeted click cyclized peptide is used for trivalent radionuclides ⁹⁰Y³⁺, ¹⁷⁷Lu^{3+,} and ⁶⁸Ga³⁺.

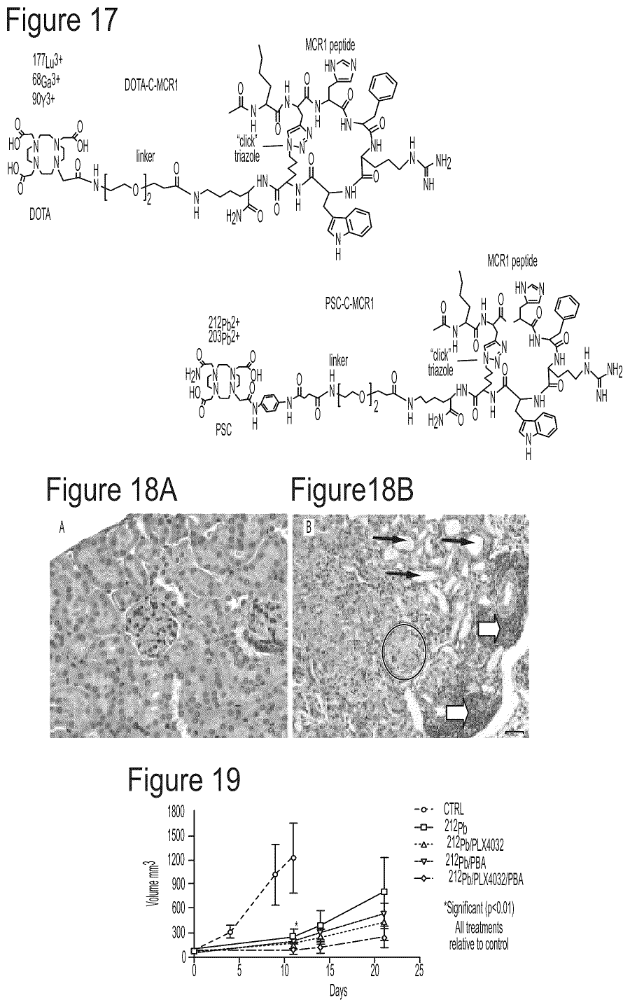

“Click”-Cyclized Peptide: The peptide backbone selected for the proposed investigation is based on a variant that was introduced previously with the addition of new evidence for improved internalization with the inclusion of a short polyethylene glycol linker between the chelator and the peptide backbone (FIG. 17). The click cyclized MCR1 peptide (DOTA-C-MCR1) demonstrated up to 16% injected dose per gram (% ID/g) of [⁶⁸Ga]DOTA-C-MCR1 with kidney accumulation of less than 5% ID/g at 90 min. post i.v. injection in mouse studies. This peptide performance represents a 7-fold improvement in tumor:kidney ratio compared to the Re-cyclized peptide used previously. The use of the Re-cyclized peptide is for comparison to the previously published alpha-particle MCR1-targeted therapy study (using B16 mouse melanoma tumors). (Leachman S A, Cassidy P B, Chen S C, Curiel C, Geller A, Gareau D, Pellacani G, Grichnik J M, Malvehy J, North J, Jacques S L, Petrie T, Puig S, Swetter S M, Tofte S, Weinstock M A. Methods of Melanoma Detection. Cancer Treat Res. 2016:167:51-105.) Thus, the DOTA-C-MCR1 (published previously) and new PSC-C-MCR1 peptides shown in FIG. 17 are used to take advantage of the observed improved tumor:kidney ratio.

### Determination of the Time/Dose Dependence of Pharmacological Enhancement of MCR1 Expression in Human Melanoma Cell Lines.

Data show that MCR1 expression can be enhanced in human melanoma cells, but the time and dose dependence are not yet known and a broader survey of cell lines is needed. Time/dose dependence are determined by RT-PCR, flow, peptide-binding, internalization, and efflux assays in 10 human melanoma cell lines (BRAF^{V600E}/BRAF^{WT}) incubated with PBA; BRAFᵢ/MEKᵢ; HDACᵢ in concentrations relevant to the in vivo setting. A total of 9 human metastatic melanoma cell lines has been selected from ATCC and Wistar Cancer Institute for these studies and include: BRAF^{V600E} SK-MEL-3, SH-4, SK-MEL-24, and BRAF^{WT} WM1361A, WM1366, WM199; and three patient derived cell lines from the University of Iowa clinics. Concentration ranges for drugs employed are selected based on the package inserts to ensure incubations are within clinically-relevant ranges. Experiments are conducted in triplicate at least twice at all combinations.

TABLE 1
Drugs and Concentrations

| Drug | Low | Medium | High | Unit |
|---|---|---|---|---|
| Vemurafenib (BRAFᵢ) | 1 | 5 | 10 | μM |
| Cobimetinib (MEKᵢ) | 0.1 | 0.5 | 1 | μM |
| PBA | 1 | 5 | 10 | μM |
| Vorinostat (HDACᵢ) | 0.5 | 5 | 10 | μM |

Quantitative Real-Time PCR (MCR1 at the mRNA Level): qPCR measurements are included to measure the change in MCR1 mRNA with changes in the concentration and incubation time for each drug and combinations (FIG. 10). These experiments are carried out as in FIG. 10 according to manufacturers' protocols, cells are seeded into 6-well plates until ˜80% confluent. After drug treatments, total RNA is isolated (Qiagen RNeasy Mini Kit). 1 μg of total RNA from each cell sample is used for reverse transcription using a high capacity reverse transcription kit (Applied Biosystem). cDNA samples are kept at −80° C. until use. Upon use, cDNA templates are employed in the qRT-PCR using a Taqman Gene Expression Assay for human MC1R (Assay ID: Hs00267167_s1). Human 18S (Assay ID: Hs99999901_s1) and human GAPDH (Assay ID: Hs03929097_g1) are used as housekeeping gene controls. The qRT-PCR reaction is perform using a Taqman Fast Universal Master Mix in a 96-well plate in 20 μL. Reactions are carried out in Applied Biosystem 7900HT. mRNA level is calculated by comparative ΔΔCt method.

Receptor Binding Assay (Functional Binding to MCR1): PCR measurements give information on the cellular response to drug treatments, but competitive binding assays convey a specific measure of changes in receptor expression (protein level) and changes in ligand-binding interactions as a result of drug treatments (FIGS. 11A-11B). After drug treatments, receptor expression is determined using synthetic α-MSH analog [¹²⁵I]-Nle⁴-D-Phe⁷-alpha-MSH ([¹²⁵I]-NDP-MSH) routinely as in FIG. 16D. (Martin M E, Sue O'Dorisio M, Leverich W M, Kloepping K C, Walsh S A, Schultz M K. “Click”-cyclized (68)ga-labeled peptides for molecular imaging and therapy: synthesis and preliminary in vitro and in vivo evaluation in a melanoma model system. Recent Results Cancer Res. 2012; 194:149-75; Baumhover N J, Martin M E, Parameswarappa S G, Kloepping K C, O'Dorisio M S, Pigge F C, Schultz M K. Improved synthesis and biological evaluation of chelator-modified alpha-MSH analogs prepared by copper-free click chemistry. Bioorg Med Chem Lett. 2011; 21(19):5757-61. PMCID: 3171621; Martin M E, Parameswarappa S G, O'Dorisio M S, Pigge F C, Schultz M K. A DOTA-peptide conjugate by copper-free click chemistry. Bioorg Med Chem Lett. 2010; 20(16):4805-7.) These experiments are replicated using [⁶⁸Ga]DOTA-C-MCR1 and [²⁰³Pb]PSC-C-MCR1.

Internalization Assay: Internalization is recognized as an important characteristic of ligand-receptor interaction for radionuclide based therapies. This is particularly important for alpha-particle therapy because internalization improves the probability of direct interaction of the alpha particle with nuclear DNA. Alpha particle interactions with DNA have a high probability of causing double strand breaks, which leads to cell death. To determine if there are changes in the internalization of MCR1, following drug treatments, internalization assays are conducted according to routine procedures. (Martin M E, Sue O'Dorisio M, Leverich W M, Kloepping K C, Walsh S A, Schultz M K. “Click”-cyclized (68)ga-labeled peptides for molecular imaging and therapy: synthesis and preliminary in vitro and in vivo evaluation in a melanoma model system. Recent Results Cancer Res. 2012; 194:149-75; Baumhover N J, Martin M E, Parameswarappa S G, Kloepping K C, O'Dorisio M S, Pigge F C, Schultz M K. Improved synthesis and biological evaluation of chelator-modified alpha-MSH analogs prepared by copper-free click chemistry. Bioorg Med Chem Lett. 2011; 21(19):5757-61. PMCID: 3171621; Martin M E, Parameswarappa S G, O'Dorisio M S, Pigge F C, Schultz M K. A DOTA-peptide conjugate by copper-free click chemistry. Bioorg Med Chem Lett. 2010; 20(16):4805-7.) Cells are washed gently with media, ([¹²⁵I]-NDP-MSH is added and the suspension is incubated for 2 h at 25° C. Binding media is aspirated and cells are rinsed and lysed in NaOH for 5 min. Cell lyses are harvested and radioactivity is measured using automatic gamma-counter (Perkin Elmer) to the determine the amount of internalized radiolabeled peptide.

Efflux Assay: Drug efflux has been implicated in the acquisition of resistance in metastatic melanoma. It is desirable that the radioligand that is internalized remains in the cell to maximize tumor cell specific radiation dose. Conducted in the same manner as internalization assays above, except fresh media is added and cells are incubated at 37° C., 5% CO₂ for 30 min, 60 min, 90 min and 180 min (n=4). At each time point, culture media are aspirated, media freshened and cell lysis are harvested and radioactivity is measured using automatic gamma-counter (Perkin Elmer). These experiments are replicated using [⁶⁸Ga]DOTA-C-MCR1 and [²⁰³Pb]PSC-C-MCR1 to determine if these differences are structural or functional MCR1 behavior changes.

CRISPR Knockouts of MCR1: An MCR1^{neg} cell line is created using the CRISPR technology and binding assays are conducted as negative controls. Briefly, A375 BRAFV600E cells are maintained in DMEM supplemented with FBS, humidified at 37° C. (5% CO₂) and routinely sub-cultured before reaching confluence by detachment with TrypLE Express (Invitrogen, Carlsbad, CA). The KN203218 MCR1 human gene knockout kit via CRISPR (containing gRNA vectors in pCAs guide) is used according to the manufacturers specifications (Origene). Recent research is revealing that CRISPR knockouts are highly specific and emerging tools are enabling an assessment of the off-target deletions. Cells are transiently transfected by calcium phosphate precipitation. Five days after transfection MCR1^{neg} cells are sorted and selected from single clones for binding assays.

### Determination If Pharmacological MCR1 Enhancement Can Maximize Tumor: Normal Accumulation of MCR1-Targeted Peptides in Mice Bearing Human Melanoma-Cell and Patient-Derived Xenografts.

Introduction: Data showed that pretreatment of mice with PBA/BRAFᵢ improved tumor response to MCR1-RT, but the optimum in vivo regimen that maximizes MCR1 expression, while minimizing radiopeptide uptake in other organs in vivo must be determined for clinical trial. Therefore in the present experiments, mice (male/female equal representation) bearing human (BRAF^{V600E}/BRAF^{WT}) melanoma tumors (6 lines ATCC/Wistar/University of Iowa patient-derived UI-PD) are pretreated with PBA/BRAFᵢ/MEKᵢ/HDACᵢ alone and in combinations, and the biodistribution of [²⁰³Pb]PSC-C-MCR1 is determined by radiometric “cut and count” methodologies at a relevant time point (4 h post injection chosen for a comparison). Data further suggested that PBA could be used to block accumulation of radiopeptide in the kidneys. Separate experiments examine this potential. BRAFᵢ and MEKᵢ are used only in experiments with the BRAF^{V600E} cell lines (SK-MEL-3, SH-4, UI-PD^{V600E}) because these drugs are not indicated for BRAF^{WT} patients. Experiments involving the BRAF^{WT} cell lines (WM1361A, WM1366, UI-PD^{WT}) are restricted to PBA and Vorinostat.

TABLE 2
Pretreatment doses for Aim 2 single agent testing of [²⁰³Pb]
PSC-C-MCR1 biodistribution in mice. Doses are
based on the recommended dose found in the USP
prescribing information.

| Drug | Dose | Unit | Daily | Route |
|---|---|---|---|---|
| Vemurafenib (BRAFᵢ) | 10 | mg/kg | twice | PO |
| Cobimetinib (MEKᵢ) | 1 | mg/kg | once | PO |
| PBA | 60 | mg/kg | twice | IP |
| Vorinostat (HDACᵢ) | 0.2 | mg/kg | once | PO |

Pretreatment Biodistribution Studies: For each experiment (single agent or combination), four time points (4 h, 1 d, 3 d, and 7 d) have been selected for pretreatment prior to the injection of the [²⁰³Pb]PSC-MCR1 to determine if receptor expression enhancement is sensitive to the duration of treatment with these drugs at clinically-relevant dosages (10 per group). Following the treatment periods, animals are injected via tail vein with [²⁰³Pb]PSC-C-MCR1. Human metastatic melanoma xenograft cells are (1-5×10⁶) subcutaneously (flank; athymic nu nu; 6-10 weeks) as described earlier. Martin M E, Sue O'Dorisio M, Leverich W M, Kloepping K C, Walsh S A, Schultz M K. “Click”-cyclized (68)ga-labeled peptides for molecular imaging and therapy: synthesis and preliminary in vitro and in vivo evaluation in a melanoma model system. Recent Results Cancer Res. 2012; 194:149-75.) Pretreatments commence when tumors reach sufficient size (˜0.2-0.3 g). Mice are administered 10 μCi (370kBq) of [²⁰³Pb]PSC-C-MCR1-peptide via tail vein and the animals are sacrificed at 4 h post injection. Blood and organs (e.g., kidney, liver, heart, lungs, etc.) are harvested, weighed, and radioactivity analyzed by routine methods (automated high-throughput gamma counter). (Martin M E, Sue O'Dorisio M, Leverich W M, Kloepping K C, Walsh S A, Schultz M K. “Click”-cyclized (68)ga-labeled peptides for molecular imaging and therapy: synthesis and preliminary in vitro and in vivo evaluation in a melanoma model system. Recent Results Cancer Res. 2012; 194:149-75) Results are corrected to % injected dose per g (% ID/g) of tissue and blood at each time point for each tissue.

Determination If PBA Reduces Kidney Accumulation with and Without Standard Amino Acid Co-Infusion: Preliminary data support the hypothesis that co-injection of PBA with [²⁰³Pb]MCR1-targeted peptides can reduce kidney accumulation of the radiopeptide (FIGS. 14A-14B), while improving tumor accumulation in mice bearing human metastatic melanoma tumors (FIG. 14B imaging; FIGS. 15A-15B therapy). The data further suggest that PBA can play a dual role in promoting cell death of BRAFᵢ-resistant melanoma and increasing the expression of MCR1 in human melanoma tumors (FIGS. 13A-13B; FIG. 14B imaging). Thus, experiments are conducted to understand the approach to co-administration of PBA with [²⁰³Pb]PSC-C-MCR1 that results in the highest tumor:kidney ratio. Preliminary published investigations of [⁶⁸Ga]DOTA-C-MCR1 achieved a tumor:kidney ratio of 3.4 at 60 m post injection of the radiopharmaceutical using a B16 mouse metastatic melanoma tumor model.

Melanoma tumors are induced as described above. Preliminary experiments demonstrated that both i.p. and i.v. pre-injections of PBA could be effective in blocking kidney accumulation of the radiopeptide. Thus, for these experiments, PBA is co-administered (i.v. and/or i.p.) ranging at dosages 30, 60, 120, 240 mg/kg from 4 h to 30 min. prior to injection of [²⁰³Pb]PSC-C-MCR1. At 2 h post injection, tumor and kidneys are harvested, weighed and assayed by standard gamma counter. Included in these experiments is an examination of the combination of PBA with a standard solution of amino acids (Arg+Lys) used clinically for patients receiving [⁹⁰Y]DOTA-tyr3-octreotide (DOTATOC) therapy, and the clinical protocol is used as a guide to minimize kidney accumulation of radiopeptides.

Determination of the Efficacy of Pharmacologically-Enhanced MCR1-RT Using [²¹²Pb] (α+φ, [¹⁷⁷Lu] (soft β), and [⁹⁰Y] (High-Energy β) in Mice Bearing Human Patient-Derived Xenografts.

Introduction: Data show that MCR1 expression can be enhanced pharmacologically in human melanoma cells (FIGS. 10, 1A-1D) and support the hypothesis that enhancing MCR1 expression improves in vivo imaging (FIGS. 15A-15C). In addition, experiments suggest that using FDA-approved drugs PBA and BRAFᵢ/MEKᵢ in combination with [²¹²Pb]MCR1 α-therapy can improve outcomes over standard of care melanoma therapy and [²¹²Pb]MCR1 α-therapy. However, a comparison with β-emitters and an assessment of the image-guided dosimetry approach is needed to select the most effective approach to advance to clinical trials. Therefore, radioactivity dose escalation and fractionated dose (30 day intervals×3) studies of [²¹²Pb]—, [¹⁷⁷Lu]—, and [⁹⁰Y]-MCR1 therapies are compared in mice (male/female) bearing human xenografts and patient derived xenografts (PDX). Tumor response and survival (up to 180 days); kidney function markers (e.g., CREA/BUN); kidney pathology IHC scoring are determined. Tumor/Kidney homogenates are analyzed for oxidative/ER stress markers (e.g., DHE oxidation; protein carbonyls; PERK). A control group (n=10) of MCR1^{neg} tumors (CRISPR; see above) are included to demonstrate receptor specific accumulation of the MCR1-targeted radiopeptides.

Dose Escalation Studies in Mice Bearing Human Melanoma Xenografts: Data established a baseline for conducting a therapeutic safety and efficacy study using single dose administrations from 100-140 μCi (3.7-5.2 MBq) (FIGS. 15A-15C) and information on potential kidney toxicity at 100 μCi (3.7MBq) (FIGS. 18A-18B) for an earlier variant of the MCR1-peptide with high kidney retention characteristics. For [⁹⁰Y]DOTA-C-MCR1 and [¹⁷⁷Lu]DOTA-C-MCR1, previous peptide targeted dose escalation studies in mice provide a template for dose escalation presently (Table 3).

TABLE 3
Dose escalation injected radioactivity doses.

| Radionuclide | Dose Settings | Unit |
|---|---|---|
| ²¹²Pb | 1 (25), 2 (50), 4 (100), | BMq (μCi) |
| (t_{1/2} 11 h) | 8 (200), 12 (300) | |
| ¹⁷⁷LU | 30 (1), 60 (1.6), 90 (205), | BMq (μCi) |
| (t_{1/2} 7 d) | 120 (3.2), 150 (4) | |
| ⁹⁰Y | 2 (50), 5 (135), 10 (270), | BMq (μCi) |
| (t_{1/2} 64 h) | 20 (540), 30 (810) | |

Each of these doses (either alone or in combination with PBA or PBA+BRAFᵢ) improved survival relative to current standard of care BRAFᵢ(vemurafenib; FIGS. 15A-15C). Animal weights and behavior were monitored throughout the study and complete responses were observed in 3 cases. A more detailed understanding of the therapeutic window for each radionuclide (²¹²Pb; ¹⁷⁷Lu; ⁹⁰Y) is established by conducting a dose escalation study in athymic nu/nu mice bearing mouse melanoma tumors (BRAF^{V600E} and BRAF^{WT} tumor cell lines); with equal gender representation (n=10 per group). Treatment response is monitored for up to 180 days post injection. Tumors are inplanted as described above and treatments initiated when tumors reach 100 mm³ as in FIGS. 15A-15C and 16A-16D. Tumor measurements are made by routine caliper protocols twice weekly as in FIG. 19. The primarily endpoints are tumor response (defined as growth rate; maximum tumor volume; FIG. 19; PLX4032=vemurafenib) and survival (defined as days to death; or to tumors having reached 1500 mm³ in size; or animals reaching a discomfort/duress endpoint according to IACUC approved protocols; FIG. 15A-15C).

Statistics. A sample size of 10 mice per group ensures at least 80% power to detect a 2.5-fold mean group difference assuming a coefficient of variation equal to 0.7. Power calculations are based on using a two-sample t-test at a single point in time with a significance level of 5%. Linear mixed effects models are used to estimate and compare group-specific tumor growth curves. The mixed effects models are expected to have higher power than the t-test since they utilize all time points. Survival curves are estimated using the Kaplan-Meier method, and compared with log-rank tests. The same methodology is employed to establish differences in normal organ toxicity parameters and causal endpoints that determine tumor response. If necessary, male and female data is pooled to increase power.

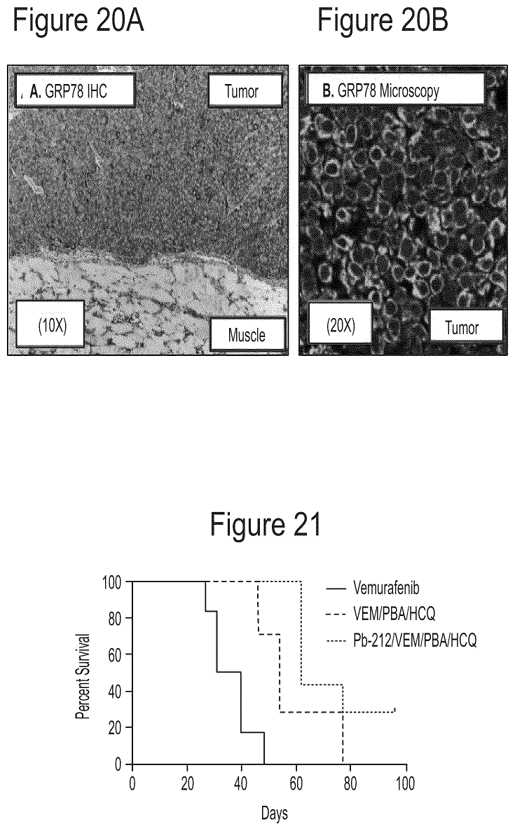

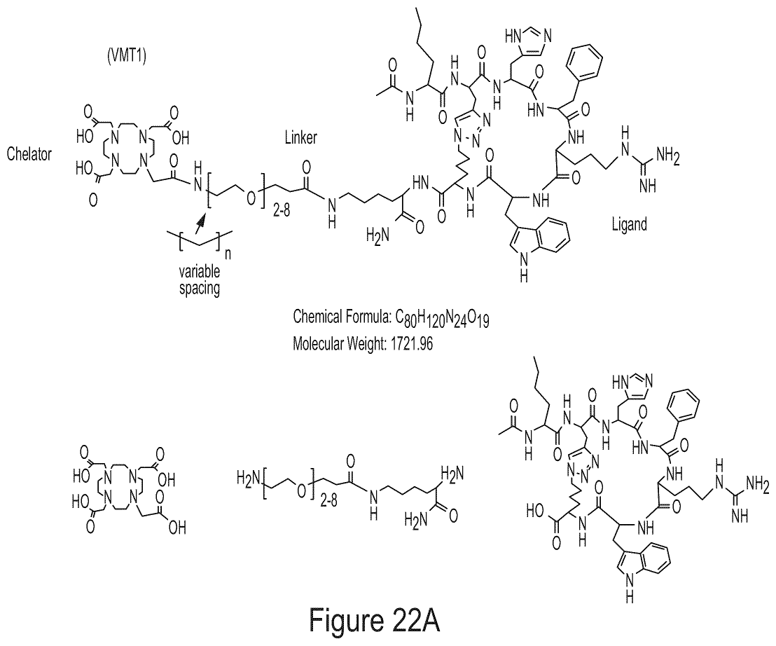

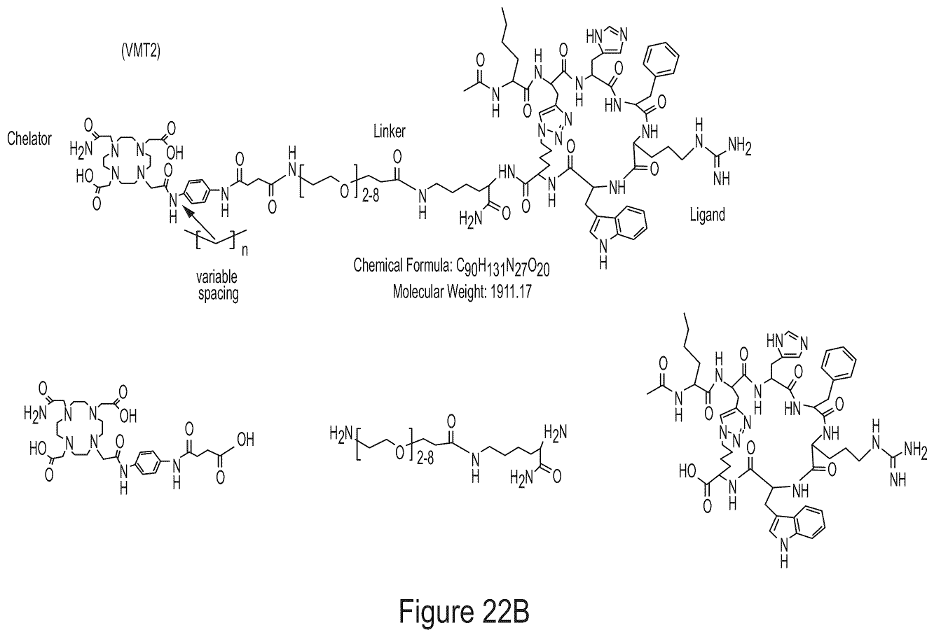

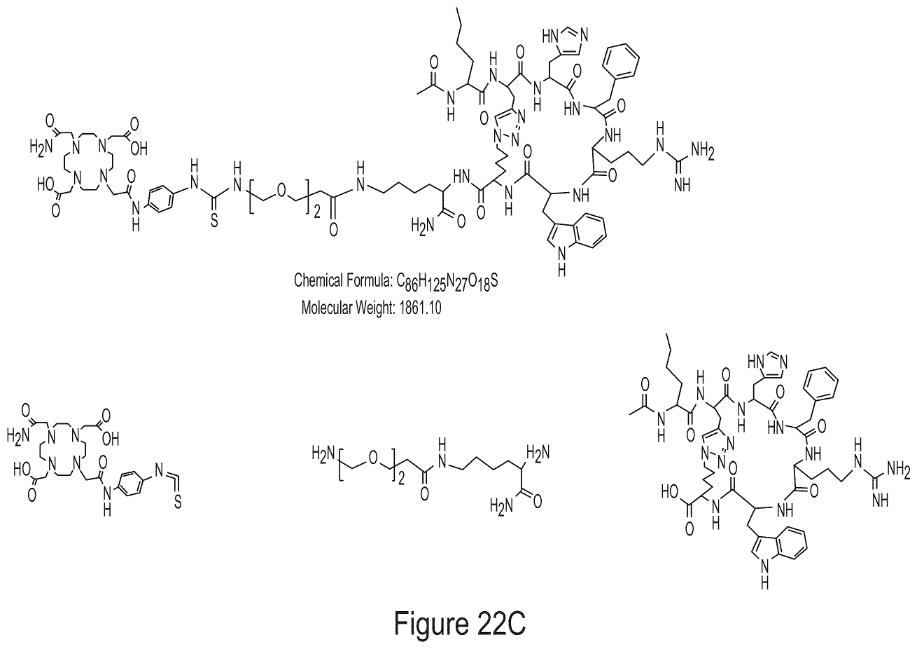

Normal Organ Toxicity Determination: Secondary endpoints renal, hepatic, and bone marrow toxicity; and other toxicities evidenced by abnormalities in a comprehensive metabolic panel (ALP, AST, ALT, creatine kinase, albumin, total bilirubin, total protein, globulin, bilirubin-conjugated, BUN, creatinine, cholesterol, glucose, calcium, phosphorus, bicarbonate TCO₂, chloride, potassium, ALB/GLOB, sodium, BUN/creatinine ratio, bilirubin-unconjugated, Na/K ratio, hemolysis index, lipemia index), and complete blood count (WBC, RBC, platelets) are also important in the collective evaluation of results. The complete panel is determined at the termination of each subject, defined by death, tumor growth to IACUC maximum (1500 mm³), or poor Body Conditioning Score (monitored daily according to the Ullman-Cullere and Foltz methodology). Renal function at 3 d, 7 d, and 30 d is evaluated by measurement of Cystatin C and BUN in serum (tail snip collection and serum analysis). At the termination of each subject, pathology is conducted using paraffin embedded kidney that has been divided into three regions (inner medulla, outmedulla, cortex). Kidney injury is measured using semi-quantitative morphological analysis in kidney sections stained with Trichrome and Periodic acid-Schiff (PAS), as well as kidney injury molecule-1 (KIM-1) expression using immunofluorescence staining and western blot protein analysis. Dihydroethidium (DHE) oxidation to its red fluorescent products by O₂^{•-} is used as a marker for steady-state levels of superoxide in cells and tissues and confirmed either using inhibition of the signal with over expression of SOD1/SOD2 or using a superoxide-specific SOD. ROS production is measured by applying DHE (see FIG. 12A) to fresh frozen kidney sections with and without GC4401 (SOD mimetic for negative control) and quantify via confocal microscopy. To confirm mitochondrial ROS production, fresh frozen kidney sections is labeled with MitSOX Red (see FIG. 12B) as well as MitoTracker Green FM (MTG). The glutathione/glutathione-disulfide (GSH/GSSG) redox couple is the most prevalent thiol redox buffer in cells, and a shift to increasing GSSG content has been shown to be an excellent marker of oxidative stress. In kidney tissue, oxidative stress parameters are measured including intracellular GSH/GSSG, 4-hydroxynonenal (4-HNE)-modified proteins (as a marker of lipid oxidation and protein damage), and NADP+/NADPH. Because oxidative-stress-induced ER stress has been implicated in tubular cell fibrosis kidney tissue is analyzed for GRP78 (FIGS. 12A-12D, FIG. 20). In addition, autophagosome formation is examined as in FIGS. 12A-12D to establish a potential role of autophagy as a progression to damage and fibrosis.

Tumor Pathology Analysis. Pathology analysis (as described above for kidney tissues) is conducted on paraffin embedded tumor at the terminus of each treatment study and includes staining for the presence of MCR1 in all tumors. Measures of oxidative stress in tumors (e.g. DHE oxidation; formation of protein carbonyls); ER stress markers (PERK, IRE1-alpha, GRP78); are combined with HNE pathology analysis and microscopy (fibrosis markers, tissue damage, autophagosome formation) to establish roles for oxidative stress, ER stress, autophagy in tumor response.

Combination Therapy Using [²¹²Pb]MCR1-Peptide with PBA and BRAFᵢ/MEKᵢ in Patient Derived Xenograft Models of Metastatic Melanoma in Mice (Single Dose Vs. Fractionated Dose): Final experiments of the optimized therapeutic combination are conducted using patient derived xenografts obtained. For these experiments, small 1-2 mm³ cubes of human tissue from a needle biopsy is transplanted via trochar into the periscapular subcutis of female immune compromised NSG mice (Jackson Labs) aged 5-8 weeks under anesthesia (2-4 mice per tumor sample, depending on amount of biopsy tissue). These are expanded until tumor size reaches ˜2,000 mm³ and then tumor is harvested after host animal has been euthanized. The excised tumor is sliced into 1-2 mm thick slices with a sterile blade under a dissecting microscope and viable tumor cubes 1-2 mm³ are isolated from necrotic tissue. These viable tumor cubes are implanted into new NSG hosts via periscapular subcutis under anesthesia and allowed to grow until a mean volume of 200 mm³ is reached at which time the PDX bearing mice are randomized into treatment groups (n=15 per group, one each for ²¹²Pb, ¹⁷⁷Lu, ⁹⁰Y). A final test of a selected dose and combination of radiolabeled MCR1-targeted peptide in the optimized radiation dose, combined with PBA, BRAFᵢ, MEKᵢ is conducted using 2 PDX models (BRAF^{V600E} and BRAF^{WT}) for each radionuclide. A single dose is compared to a fractioned dose of the same radioactivity injection at 21 d intervals (based on FIGS. 15A-15C, FIG. 19) for each radionuclide. Tumor and normal organ analysis is conducted at the termination of each subject as described above. MCR1 pathology is conducted before implantation and at the termination of each study. A select number of control animals is implanted and euthanized to examine tumor for MCR1 at time points of 7 d, 14 d, 28 d of each cell line (n=5 per group).

Image-Guided Therapy Evaluation and Dosimetry: One of the potential benefits of the [²⁰³Pb]MCR1-peptide (SPECT/CT) image-guided [²¹²Pb]MCR1-peptide therapy is the ability of the imaging scan to provide quantitative dosimetry information that can be used to select patients who can benefit; and to be able to use the imaging information to develop a dosimetry plan. Thus, a comparison of the dosimetric information obtained from [²⁰³Pb]PSC-C-MCR1 imaging scans performed in the Small Animal Imaging Core is conducted on a control group of 10 PDX therapy candidates described above in advance of the therapy. Following the therapy studies, these images and associated data are examined.

Migration of ²¹²Pb Daughter ²¹²Bi from Parent Radionuclide in the In Vivo Setting. Due to the recoil energy of the ²¹²Pb-alpha decay, daughter radionuclides ²¹²Bi, ²¹²Po, ²⁰⁸T1 are released from the chelator and are free to interact biochemically. In parallel experiments (for half-life considerations), human metastatic melanoma tumor bearing mice (as above) are injected with 200 μCi (7.4 MBq) of [²¹²Pb]PSC-C-MCR1 and a biodistribution study is conducted at 1 h and 4 h post injection in which the tissues are analyzed by high resolution gamma-ray spectroscopy using a High-Purity Germanium Gamma Spectrometer (HPGe) for the gamma-ray spectra of each radionuclide. The gamma spectra of ²¹²Pb and its daughters are distinguishable spectroscopically. Although there are limitations in the number of animals that can be analyzed in a given scenario, a single animal is included for these analyses with each therapy session. Biodistribution studies are conducted in which critical organs are analyzed to determine the concentration of unsupported vs. supported ²¹²Bi in each sample to develop a detailed understanding of this potential non-targeted dose to consider in dosimetric analysis. ²¹²Bi is used as a measure of other radionuclides because the half-lives of ²¹²Po and ²⁰⁸T1 are very short and controlled by the ²¹²Bi biodistribution.

### Example 4

FIG. 21. Survival of mice bearing human metastatic melanoma xenografts (A375) treated with a single dose (i.v.) of [²¹²Pb]DOTA-MCR1, shown as ²¹²Pb (˜100 μCi) with and without a combination of BRAFᵢ (vemurafenib 10 mg/kg b.i.d); PBA (120 mg/kg i.p.); and hydroxychloroquine. Treatments were standardized to begin when tumors reach 100 mm³. Mice were euthanized according to IACUC protocols (when tumors reached 1500 mm³ or ulceration appeared) or at about 100 d. These data support the hypothesis that [²¹²Pb]DOTA-MCR1 therapy has the potential to improve outcomes for metastatic melanoma patients relative to standard of care therapy.

### Example 5

### Materials and Methods

### Cell Lines, Reagents, Materials, and Animals

B16-F10, B16-F0, YUMM1.7 cells were obtained from ATCC and used within passage 10. All cells were culture in complete growth media including DMEM medium with 10% FBS, 100 units/mL Pen Strep, and 100 units/mL streptomycin. All cells were grown at 37° C. in a humidified atmosphere (5% CO₂). Radiometals ²⁰³Pb chloride was obtained from Lantheus Medical Imaging (North Billerica, MA, USA). The ²²⁴Rₐ/²¹²Pb generator was provided by Oak Ridge National Laboratory (Oak Ridge, TN, USA). Pb-specific resin was obtained from Eichrom Technologies (Lisle, IL, USA). Anti-mouse CTLA-4 (Clone 9H10), anti-mouse PD-1 (Clone 29F.1A12), and rat IgG2a isotype control were purchased from BioXCell (Lebanon, NH, USA). Fluorophore-conjugated antibodies used in FACS were purchased from Biolegend (San Diego, CA, USA). Matrigel was purchased from Corning (Corning, NY, USA). StrataX C-18 SPE cartridges were obtained from Phenomenex (Torrance, CA, USA). All other chemicals were purchased from Thermo Fisher Scientific (Waltham, MA, USA). C₅₇BL6 mice and Rag1 KO mice were obtained from The Jackson Laboratory (Bar Harbor, ME, USA). Athymic nude mice were purchased from Envigo (Indianapolis, IN, USA). All animal studies were performed in accordance with the Guide for the Care and Use of Laboratory Animals.

### Radiolabeling, In Vivo Biodistribution and Kidney Dosimetry

To determine the injected radioactivity of [²¹²Pb]VMT01, [²⁰³Pb]VMT01 was used as a surrogate in to determine the biodistribution of Pb-labeled VMT01. Radiolabeling of VMT01 was carried out according to published methods. Briefly, ²⁰³Pb²⁺ was purified on 50 mg Pb-resin (Eichrom Technologies, Lisle, IL, USA) and eluted into reaction vessel using 0.5 M sodium acetate (NaOAc) pH=6 buffer. The reaction vessel contained 20 nmole VMT01 peptide precursor and 0.29 mL of 0.5 M sodium acetate (NaOAc) pH=4 buffer to adjust final pH to 5.4. The reaction solution was heated under 85° C. for 30 min. After reactions, free ²⁰³Pb was removed by StrataX C-18 SPE cartridge (Phenomenex, Torrance, CA, USA) and final products were collected in 50% EtOH in saline. Biodistribution of [²⁰³Pb]VMT01 was determined in a B16-F10 murine melanoma xenograft model in athymic nude mice. B16-F10 xenograft was developed by the subcutaneous (SC) injection of 2×10⁵ B16-F10 cells at the left shoulder in 100 μL 50% Matrigel in complete growth media. Then, 74 KBq of [²⁰³Pb]VMT01 (5.4 pmole) were administered via tail vein injection (2 male and 2 female at each time point) in 100 μL of saline with less than 10% EtOH content. At 0.5, 1.5, 3, 6, and 24 h post-injection, animals were euthanized, and organs of interest were harvested and weighed. Radioactivity in tumor and organs was measured on a Cobra II automated gamma counter. To determine the injected radioactivity of [²¹²Pb]VMT01, time-integrated accumulation of radioactivity in kidney was calculated by the trapezoidal method up to 48 h accounting for approximately 5 half-lives of ²¹²Pb (t_{1/2}=10 h). The value for 48 h was extrapolated from the last three time points (3, 6, and 24 h) of the biodistribution by one phase exponential decay with least squares fitting method (GraphyPrism V7). The average kidney volume was assumed to be 0.33 cm³ for C57BL6 mice. The DigiMouse voxel phantom model (28 g; normal male mouse) was used to calculate s-value and absorbed dose in kidney from the decay of ²¹²Pb using the Particle and Heavy Ion Transport code System (PHITS) software version 2.76 (Japan Atomic Energy Agency, Tokai, Japan). The voxel size of the model was adjusted to have the same kidney volume as the averaged value (0.33 cm³). The elemental composition of the kidney and the mass density was assumed to be identical as the human adults' values obtained from the International Commission on Radiation Units and measurements (ICRU) report. The injected radioactivity of [²¹²Pb]VMT01 was determined using 11 Gy dose deposition in the kidney as threshold in this study as guided by a previous safety study of [²¹³Bi]DOTATATE.

### Combination Therapy of Immune Checkpoint Inhibitors and [²¹²Pb]VMT01

Cooperative anti-tumor efficacy between ICIs and [²¹²Pb]VMT01 was determined in C57BL6 mice bearing B16-F10 melanoma. Preparation of [²¹²Pb]VMT01 was described in our previous publication. In general, ²¹²Pb²⁺ was eluted from ²²⁴Rₐ/²¹²Pb generator (US Department of Energy, Oak Ridge, TN, USA) with 2 M HCl. The ²¹²PbCl₂ eluate was purified on Pb-resin and reacted with 20 nmole VMT01 as described above. After reactions, free ²¹²Pb²⁺ was removed by C-18 SPE cartridge and a final dose was collected in 50% EtOH in saline. In C57BL6 mice bearing a B16-F10 tumor, therapies were initiated when the tumor size reached 50 mm³ (4-5 days post-inoculation). For [²¹²Pb]VMT01 monotherapy, 4.1 MBq [²¹²Pb]VMT01 (0.3 nmole) was administered via the tail vein in 100 μL of saline containing 8 mg of DL-lysine to further reduce the accumulated radiation dose in kidney. ICIs including 200 μg of anti-mouse CLTA4 and 200 μg anti-mouse PD-1 were administered twice a week via IP injection. The combination of ICIs and [²¹²Pb]VMT01 was administered concurrently on day 0, followed by routine doses of ICIs given twice a week via IP injection. Control animals were treated with 200 μg rat IgG2a isotype control via IP injection. Upon conclusion of the study, tumor re-challenge was conducted in animals that demonstrated complete tumor regression as results from combination of [²¹²Pb]VMT01+ICIs. These animals were removed from study on 80 days and kept in animal housing facility for 7 days, followed by tumor re-challenge using SC injection of 50,000 naïve B16-F10 cells on Day 87. Animals were monitored for extra 60 days post-inoculation.

To determine the impact of dosing regimen of [²¹²Pb]VMT01 on the effectiveness of [²¹²Pb]VMT01 as monotherapy as well as in combination with ICIs, total 4 MBq[²¹²Pb b]VMT01 was delivered via tail vein injection over three fractions within 6 days (n=7), including 2 MBq on day 0, 1 MBq on day 3, and 1 MBq on day 6. Each fraction of [²¹²Pb]VMT01 was administered in 100 μL of saline containing 8 mg of DL-lysine. Combination of [²¹²Pb]VMT01 and ICIs started concurrently on day 0, by IP injection of 200 μg anti-mouse CLTA4 and 200 μg anti-mouse PD-1, along with the first 2 MBq fraction of [²¹²Pb]VMT01. Control and ICIs monotherapy cohorts were treated with IP injection of IgG isotype control and anti-mouse CLTA4/anti-mouse PD-1, respectively, as described above. Tumor growth was monitored by measuring tumor size twice a week by length (L) and width (W) using the following equation:

$$Volume = L \times {W}^{2} / 2$$

Animals were removed from the study when tumor size reached 1500 mm³; tumor ulcerations appeared; body weight loss was more than 20% compared with initial weight; or other significant toxicity was observed. To evaluate the effectiveness of treatments in each cohort, median overall survival (MOS) and tumor-doubling time were compared with initial tumor size on day 0.

### Combination of Immune Checkpoint Inhibitors and Single Dose of [²¹²Pb]VMT01 in Rag1 KO Mice

To investigate if the immunogenicity of [²¹²Pb]VMT01 was mediated by adaptive immune response, the combination of [²¹²Pb]VMT01 and ICIs was applied to B6.129S7-Rag1^{tmlMom}/J mice (i.e., Rag1 KO mice) mice. Due to the genetic modification, Rag1 KO mice do not produce mature B and T lymphocytes therefore are considered “non-leaky” immune deficiency. B16-F10 melanoma xenograft was developed in Rag1 KO mice by SC injection of 2×10⁵ of B16-F10 cells on left shoulder. Therapies were initiated when tumor size neared 50 mm³. Monotherapy of [²¹²Pb]VMT01 was delivered as single injection of 4.1 MBq [²¹²Pb]VMT01 via tail vein. ICIs (i.e., 200 μg anti-CTLA-4 and 200 μg anti-PD-1) were administered via IP injection twice a week. Combination of ICIs and [²¹²Pb]VMT01 was administered concurrently on day 0. Control cohorts were treated with IP injection of IgG isotype control antibody twice a week. Following the treatments, tumor size was measured twice a week by length (L) and width (W) as described above.

### Vaccination and Tumor Re-Challenge

To determine the activation of anti-tumor immune response by [²¹²Pb]VMT01, [²¹²Pb]VMT01 treated melanoma cells were injected in C57BL6 mice as cell-based vaccine to stimulate anti-tumor immunity. B16-F10 and B16-F0 cells were kept under 37° C. and 5% CO₂ to grow until 50-80% confluency in 60 mm petri dishes. The 0.6 MBq [²¹²Pb]VMT01 was added to 5 mL total growth media and incubated for 24 h before removal of radioactive media. After treatment, cells were cultured in fresh media for another 24 h before further inoculation in C57BL6 mice. Upon vaccination in C57BL6 mice, 2×10⁶ [²¹²Pb]VMT01 treated B16-F10 or B16-F0 cells were subject to SC inoculation in 100 μL 50% Matrigel in total culture media at the left shoulder (n=7-8). In control animals, in 100 μL 50% Matrigel in total culture media without any cells, SC was injected at the left shoulder. Then, 7 days post-vaccination, mice were re-challenged with SC inoculation of 50,000 naïve B16-F10 or B16-F0 at the contralateral right shoulder. Tumor progression was monitored by measuring length (L) and width (W) twice a week.

### Generation of Immunosensitized Syngeneic Melanoma Cells by [²¹²Pb]VMT01

The immunogenicity of [²¹²Pb]VMT01 was determined in immunotolerant syngeneic mouse melanoma cells lines B16-F10 and YUMM1.7. Immunosensitized melanoma cells were generated from B16-F10 and YUMM-1.7 cells using modified methods. YUMM-PR (post radiation) and B16-PR cells were generated by treating naïve YUMM-1.7 and B16-F10 cells with 0.22 MBq [²¹²Pb]VMT01 for 24 h in complete growth media (DMEM medium with 10% FBS, 100 units/mL Pen Strep, and 100 units/mL streptomycin) in 35 mm petri dishes. After [²¹²Pb]VMT01 treatment, YUMM-PR and B16-PR cells were cultured under 37° C. and 5% CO₂ in complete culture media for extra 2 weeks, allowing for full recovery of irradiated cells. Culture media were replaced every three days to remove floating cells. After two weeks, xenografts of YUMM-PR and B16-PR tumor were developed by SC inoculation of 1×10⁶ YUMM-PR and 1×10⁵ B16-PR cells in female C57BL6 mice (n=5) as described above. ICIs treatment (i.e., 200 μg anti-mouse CLTA-4 and 200 μg anti-mouse PD-1) was initiated when YUMM-PR and B16-PR tumors reached 100 mm³ and 50 mm³, respectively. ICIs and rat IgG isotype control were administered via IP injection twice a week.

### FACS Analysis of Tumor Infiltrating Lymphocytes

[²¹²Pb]VMT01-induced tumor infiltrating lymphocytes (TIL) in B16-F10 was analyzed by FACS. In C57BL6 mice bearing B16-F10 melanoma, 1.4 MBq of [²¹²Pb]VMT01 was injected via tail vein (n=4) when tumor size reached 100 mm³. Control animals were treated with isotonic saline (n=5). Then, 7 days after treatments, animals were euthanized, and tumors were exercised for FACS analysis. Briefly, tumor samples were placed in GentleMACS™ C-tubes (Miltenyibiotec) containing 3 mL of ice-cold RMPI media. Tumor samples were homogenized on gentleMACS™ Dissociator (Miltenyibiotec) and filtered through 70-micron cell strainer to get single cell suspension. Then, 15 mg of homogenized samples was transferred to 12 75 mm tubes and washed twice with ice-cold PBS. Cells were stained for live/dead using Zombie Aqua dye diluted at 1:100 in 100 μL PBS and incubated at room temperature for 15 min. To stain surface markers, cells were first washed in FACS buffer (PBS, 2% BSA, 1 mM EDTA, 0.1% sodium azide) twice and then staining in 100 μL of FACS buffer containing 0.5-1 μg of anti-mouse CD45-PerCP-Cy5 (103132, Biolegend, San Diego, CA, USA), anti-mouse CD3-APC (100235, Biolegend), anti-mouse CD19-PE-Cy7 (115519, Biolegend), anti-mouse CD4-APC-Cy7 (100413, Biolegend), and anti-mouse CD8-FITC (100706, Biolegend). Cells were incubated under room temperature for 15 min before washed with FACS buffer twice. Finally, stained cells were fixed in 200 μL 0.5% formaldehyde and analyzed on a BD Becton Dickinson LSR II (VA Satellite Lab) flow cytometer at the Flow Cytometry Facility at the University of Iowa.

### Results

### Radiolabeled Peptide VMT01 Delivers Ionizing Radiation to Melanoma Cells via Specific Binding to MC1R

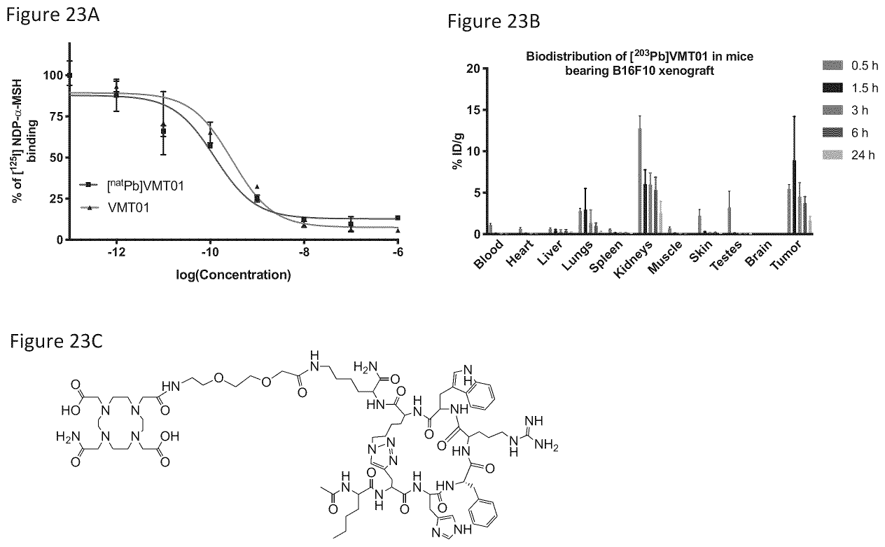

Radiolabeled synthetic α-MSH analog VMT01 (FIGS. 23A,23C) was employed to deliver Pb isotopes ²⁰³Pb and ²¹²Pb to melanoma cells via binding with MC1R. Competitive binding assays against [¹²⁵I]NDP-α-MSH were conducted in B16-F10 to determine the binding affinity of VMT01 and [^{nat}Pb]VMT01. Further, 0.29 and 0.15 nM IC₅₀ were identified for VMT01 and [^{nat}Pb]VMT01, respectively (FIG. 23A). In vivo biodistribution of Pb-labeled VMT01 was determined using [²⁰³Pb]VMT01 in female athymic nude mice bearing MC1R-positive B16-F10 melanoma. Rapid accumulation of [²⁰³Pb]VMT01 in B16-F10 melanoma was observed (FIG. 23B). Accumulation of [²⁰³Pb]VMT01 in B16-F10 tumors was 5.5, 8.9, 4.5, 3.8, and 1.7 percent injection dose per gram (% ID/g) at 0.5, 1.5, 3, 6, and 24 h post administration, respectively (Table 4). Excessive [²⁰³Pb]VMT01 was cleared from circulation rapidly, with 1.1% ID/g residual radioactivity in blood at 0.5 h post-injection (FIG. 23B, Table 4). Off-target accumulation was primarily localized in kidney, with 12.8, 6.1, 6.0, 5.3, and 2.6% ID/g at 1.5, 3, 6, and 24 h post injection (FIG. 23B, Table 4). Cumulative radioactive decays of [²¹²Pb]VMT01 in kidney was integrated using the biodistribution data of [²⁰³Pb]VMT01 and corrected with decay half-life of ²¹²Pb (t_{1/2}=10.64 h). The calculated s-value for [²¹²Pb]VMT01 in kidney was 2.84E-06 Gy/Bq-s in kidney. To maintain the dose deposition in kidney. To maintain the dose deposition in kidney below 11 Gy for therapeutic application, the upper limits of injected radioactivity for [²¹²Pb]VMT01 were estimated to be 4/1 mBq.

TABLE 4
Biodistribution of [²⁰³Pb]VMT01 in B16-F10 melanoma xenograft model.

| | 0.5 h | | 1.5 h | | 3 h | | 6 h | | 24 h | |
|---|---|---|---|---|---|---|---|---|---|---|
| | | Std. | | Std. | | Std. | | Std. | | Std. |
| | Average | Dev | Average | Dev | Average | Dev | Average | Dev | Average | Dev |
| Blood | 1.12 | 0.34 | 0.08 | 0.02 | 0.03 | 0.01 | 0.02 | 0.01 | 0.01 | 0.00 |
| Heart | 0.66 | 0.30 | 0.08 | 0.03 | 0.05 | 0.01 | 0.03 | 0.01 | 0.02 | 0.00 |
| Liver | 0.64 | 0.25 | 0.43 | 0.12 | 0.31 | 0.19 | 0.37 | 0.17 | 0.22 | 0.06 |
| Spleen | 0.58 | 0.14 | 0.17 | 0.01 | 0.12 | 0.03 | 0.11 | 0.04 | 0.09 | 0.01 |
| Lungs | 2.80 | 0.66 | 2.98 | 2.55 | 1.34 | 1.57 | 1.00 | 0.34 | 0.20 | 0.16 |
| Kidneys | 12.78 | 2.97 | 6.06 | 1.72 | 5.98 | 1.38 | 5.32 | 1.55 | 2.59 | 1.39 |
| Tumor | 5.47 | 1.05 | 8.90 | 6.30 | 4.50 | 1.71 | 3.76 | 0.77 | 1.68 | 0.46 |
| Muscle | 0.73 | 0.29 | 0.09 | 0.04 | 0.04 | 0.02 | 0.03 | 0.01 | 0.02 | 0.00 |
| Skin | 2.23 | 1.49 | 0.27 | 0.05 | 0.15 | 0.03 | 0.17 | 0.06 | 0.09 | 0.01 |
| Brain | 0.06 | 0.02 | 0.02 | 0.01 | 0.01 | 0.00 | 0.01 | 0.00 | 0.01 | 0.00 |
| Testes | 3.23 | 3.93 | 0.10 | 0.04 | 0.04 | 0.01 | 0.04 | 0.01 | 0.03 | 0.01 |

### Combination of ICIs and [²²¹Pb]VMT01 Induces Significant Tumor Inhibition and Lasting Anti-Tumor Immunity

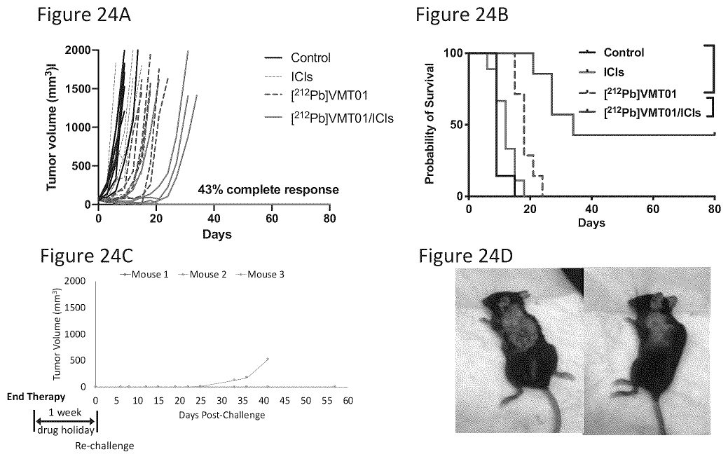

To determine the potential cooperative anti-tumor effects that could be induced by combining MC1R-targeted α-TRT and ICIs, [²¹²Pb]VMT01 was administered as a monotherapy or in combination with dual ICIs (i.e., anti-CLTA-4+anti-PD-1) in immunocompetent C57BL6 mice bearing B16-F10 syngeneic murine melanoma tumors (n=7 in each cohort). Tumors were induced by a subcutaneous inoculation of 2×10⁵ B16-F10 cells on the left shoulder. Therapies were initiated when tumors reached 60±13 mm³. In the control cohort, tumor-volume endpoint (1500 mm³) was reached shortly after the initiation of the experiment (<10 days). Further, 86% of animals that received IgG isotype control were removed from study within 10 days due to uncontrolled tumor growth (FIG. 24A). The median overall survival (MOS) of control animals was nine days (FIG. 24B). Dual ICIs injected twice a week did not provide significant control on tumor growth, consistent with these tumors being “immunologically cold.” The MOS (12 days) in the ICIs alone treatment group was not significantly different from the control group (FIG. 24B). Median tumor-doubling time was not identified in these two groups due to rapid uncontrolled tumor growth. On the other hand, a single injection of 4.1 MBq [²¹²Pb]VMT01 significantly suppressed the growth of B16-F10 tumor in all treated animals. In these animals, it took median 10 days to reach doubled tumor size compared with tumor size day 0 (FIG. 24A). The MOS was also extended to 18 days (FIG. 24B, p<0.0001 vs. control). More significant inhibition of tumor growth was observed in mice treated with a combination of [²¹²Pb]VMT01 and ICIs. In this cohort of animals, the treated tumors took 24 days to reach doubled size from day 0 (FIG. 24A). MOS was also prolonged to 34 days (FIG. 24B, p<0.001 vs. [²¹²Pb]VMT01 monotherapy). It is important to note that 100% (seven in seven) animals responded to this combination therapy, with 43% (three in seven) showing complete tumor regression and the surviving mice remained tumor-free until the conclusion of the experiment on day 80. No weight loss or other significant toxicity was observed in these animals.

After the conclusion of therapy, adaptive anti-tumor immunity was determined in animals that had achieved complete responses. These mice were re-challenged by SC inoculation of 50,000 naïve B16-F10 cells after conclusion of the therapy study (one week ICIs drug holiday). Remarkably, while control B16-F10 tumors are generally aggressive and grow rapidly, inoculations of the naïve B16-F10 cells in these animals were either significantly attenuated or did not grow within the study period. Of the three mice in this cohort, two animals completely rejected tumor inoculation and maintained tumor-free status for an additional 60 days. Further, tumor development was significantly attenuated in the third mouse in this cohort, with the tumor slowly developing and emerging approximately 30 days after implantation (FIGS. 24C-24D). These data suggest that [²¹²Pb]VMT01 and ICIs combine to induce a cooperative tumor-inhibition effect that can lead to complete tumor regression, where monotherapies of ICIs and [²¹²Pb]VMT01 fall short. In addition, the anti-tumor immunity acquired during the course of the combination therapy immunizes the mice to reject further tumor implantation or to significantly inhibit tumor growth.

Combination of ICIs with Fractionated [²¹²Pb]VMT01 Compromised the Cooperative Anti-Tumor Effects Observed for the Single-Dose α-TRT Plus ICIs Combination

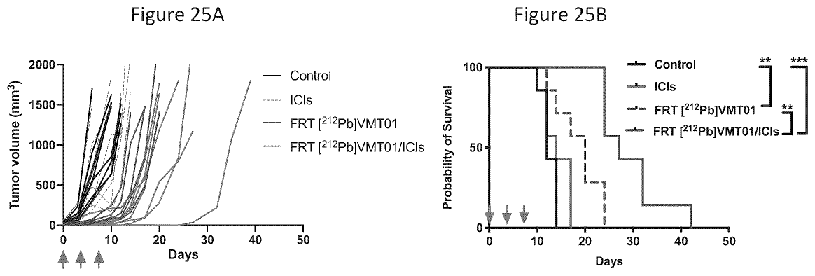

To refine the understanding of the combination of [²¹²Pb]VMT01 and ICIs, the impact of dosing regimen of [²¹²Pb b]VMT01 as monotherapy and in combination with ICIs was examined. For this assessment, [²¹²Pb]VMT01 was administered using a dosing regimen of a total 4.0 MBq over three fractions (2+1+1 MBq) injected in C57BL6 mice via tail vein (n=7), with an interval of three days between each administration. The [²¹²Pb]VMT01 was administered as monotherapy, as well as in combination with ICIs. Despite that [²¹²Pb]VMT01 was administered over three fractions, fractionated [²¹²Pb]VMT01 monotherapy resulted in robust inhibition of B16-F10 tumor growth. Compared with the rapid tumor growth in control and ICIs cohorts, it took 12 days to reach doubled tumor size in mice treated monotherapy of fractionated [²¹²Pb]VMT01 (FIG. 25A). The MOS in animals administered with fractionated [²¹²Pb]VMT01 was improved to 20 days (p<0.05 vs. control, FIG. 25B). When the fractionated [²¹²Pb]VMT01 regimen was applied in combination with ICIs, a clear cooperation between [²¹²Pb]VMT01 and ICIs was observed. In these animal cohorts, the median tumor-doubling time was extended to 17 days, and MOS was also extended to 27 days (FIGS. 25A-25B). Compared with monotherapies of fractionated [²¹²Pb]VMT01 or ICIs, the improvement from combination of fractionated [²¹²Pb]VMT01 and ICIs was significant (p<0.01 vs. [²¹²Pb]VMT01; p<0.001 vs. ICIs). However, all animals treated with combination therapy eventually developed progressive tumors and no complete tumor regression was observed (FIG. 25B). These results indicate that both single and fractionated injection of [²¹²Pb]VMT01 monotherapy efficiently attenuated MC1R-postive melanoma tumor, but only single injection of [²¹²Pb]VMT01 induced an immune response that led to complete tumor regression in combination with ICIs.

### [²¹²Pb]VMT01 Induces Anti-Tumor Immunity That Relies on the Involvement of Adaptive Immunity

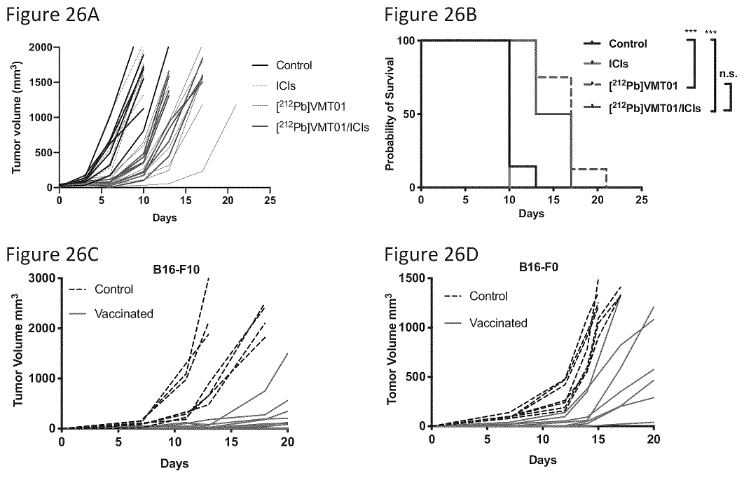

To begin testing whether T cell maturation was necessary for the cooperative anti-tumor effect of the combination of [²¹²Pb]VMT01 and ICIs, this treatment combination was administered to B6.129S7-Rag1^{tmlMom}/J mice (i.e., Rag1 KO mice) bearing B16-F10 tumors (n=7), where 4.1 MBq [²¹²Pb]VMT01 was administered as a single injection on day 0, as this regimen showed most significant anti-tumor effectiveness. Not surprisingly, animals in control and ICIs monotherapy cohorts rapidly reached endpoint (1500 mm³) due to aggressive tumor progression. Within 10 days, 100% animals in ICIs cohorts and 86% animals in control cohorts were removed (FIG. 26A). On the other hand, despite the depleted adaptive immunity in Rag1 KO mice, monotherapy of 4.1 MBq [²¹²Pb]VMT01 still led to significant inhibition of growth of B16-F10 tumors (FIG. 26A) and the MOS in these animals was improved to 17 days (FIG. 26B). However, with the deficient adaptive immunity in the Rag1 KO mice, the benefit from combination of [²¹²Pb]VMT01 and ICIs was completed abrogated. Compared with [²¹²Pb]VMT01 monotherapy, combination therapy did not provide a significant improvement in therapeutic outcome (MOS=15 days, p>0.05 vs. [²¹²Pb]VMT01, FIGS. 26A-26B). These data indicate that the immunogenicity of [²¹²Pb]VMT01 and anti-tumor cooperation with ICIs require intact adaptive T cell immunity.

To elucidate the activation of tumor-specific immune response by [²¹²Pb]VMT01, in vivo vaccination and tumor re-challenge assays were performed. Female C57BL6 mice (n=7) were vaccinated by SC inoculation of 2×10⁶ B16-F10 or B16-F0 cells that were pretreated with 0.6 MBq [²¹²Pb]VMT01 in vitro. These [²¹²Pb]VMT01-treated melanoma cells were employed as a cell-based vaccine in C57BL6 mice. Control animals were injected with 100 μL PBS subcutaneously. [²¹²Pb]VMT01 treatment efficiently killed melanoma cells, as implantation of 2×10⁶ [²¹²Pb]VMT01-treated B16-F10 and B16-F0 cells did not give rise to any tumor growth (FIGS. 26C-26D). One week post vaccination, both immunized mice and control mice were re-challenged by SC inoculation of 50,000 naïve B16-F10 or B16-F0 cells on the contralateral side of animals. Compared with the control mice, slower progression of both B16-F10 (FIG. 26C) and B16-F0 tumors (FIG. 26D) was observed in vaccinated mice, indicating that [²¹²Pb]VMT01 activates tumor-specific immunogenicity that produces immune protection against further tumor inoculation.

### [²¹²Pb]VMT01 Sensitizes Immunotolerant Melanoma Cells to ICIs and Induces Tumor-Infiltrating Lymphocytes

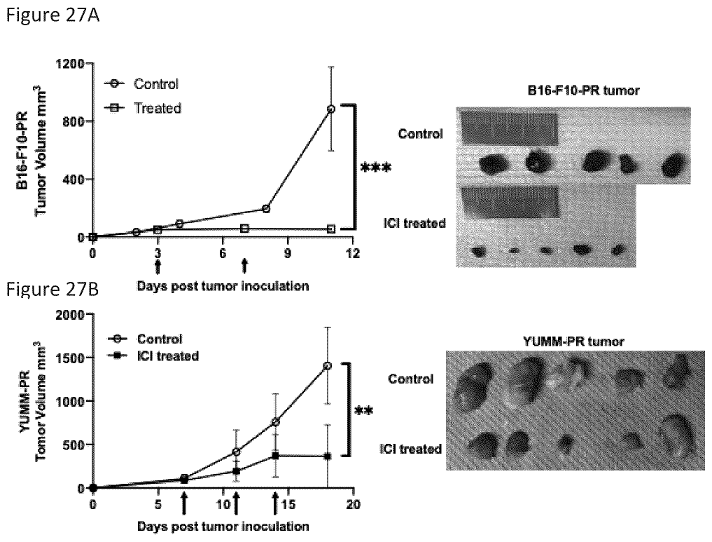

To determine if [²¹²Pb]VMT01 changes the immunophenotype of melanoma cells, sensitization to ICIs by [²¹²Pb]VMT01 was conducted in immunotolerant B16-F10 and YUMM1.7 syngeneic melanoma cells. Due to their immunotolerant nature, B16-F10 tumor did not respond to ICIs treatment as we demonstrated above. Similarly, the immunotolerance of YUMM1.7 tumor has been previously characterized, whereas UV radiation treatment induced accumulation of somatic mutations that sensitized YUMM1.7 tumor to ICIs treatment. In this study, B16-PR (post radiation) and YUMM-PR cells were generated by treating these cells with [²¹²Pb]VMT01 in vitro. After SC implantation, fast tumor growth was observed in both B16-PR and YUMM-PR tumors in female C57BL6 mice (n=5). For B16-PR tumor, in mice administered with rat IgG isotype control antibody, tumor size reached 884±324 mm³ within 11 days post inoculation (FIG. 27A). For comparison, this growth rate was almost identical to naïve B16-F10 tumors in C57BL6 mice (FIG. 26C), indicating the B16-PR cells had recovered from [²¹²Pb]VMT01 treatment upon SC inoculation and were capable to give rise to fast-growing tumors. To determine if the B16-PR tumors are responsive to ICI treatments, mice were administered an identical ICIs therapy regimen as described for previous experiments. In this case, IP injection of ICIs significantly compromised the tumor growth of B16-PR, with average tumor size 56±20 mm³ on day 11 (FIG. 27A, p<0.001 vs. control B16-PR). Similarly, with the injection of rat IgG control, YUMM-PR tumor grew to 1404±438 mm³ within 18 days post inoculation (FIG. 27B), whereas ICIs treatment significantly suppressed tumor growth of YUMM-PR tumor (FIG. 27B, 361±364 mm³, p<0.01 vs. YUMM-PR control). These data indicate that [²¹²Pb]VMT01 treatment sensitized these immunotolerant syngeneic melanoma cells to ICIs treatment.

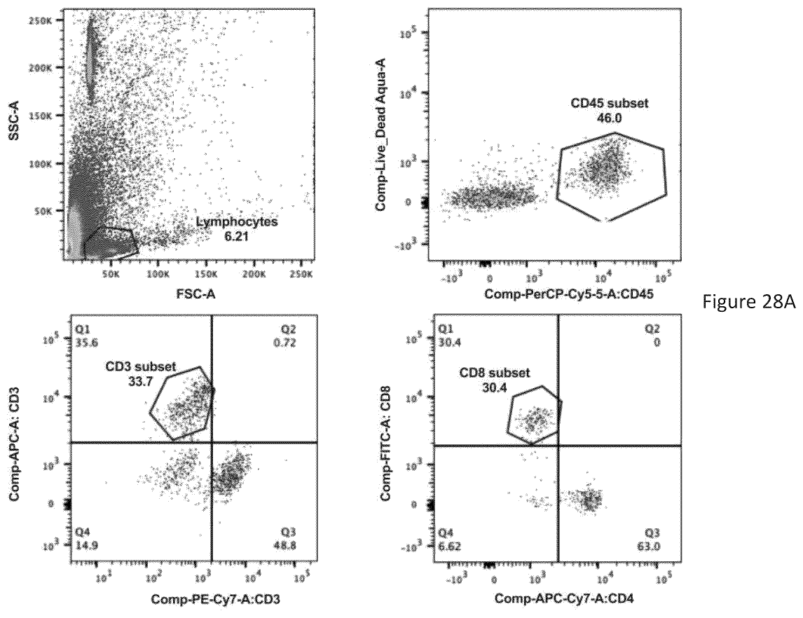

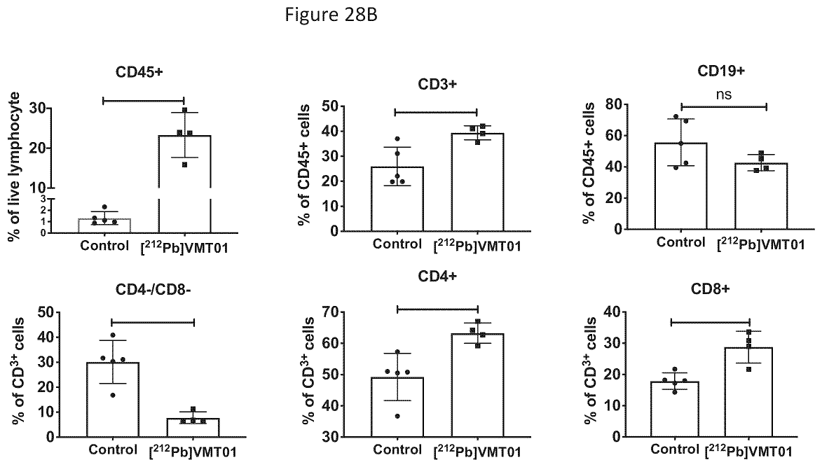

To develop a more detailed understanding of the tumor-specific immune response to [²¹²Pb]VMT01, changes in tumor-infiltrating lymphocytes (TILs) was measured in [²¹²Pb]VMT01 treated B16-F10 tumors. For these experiments, C57BL6 mice bearing B16-F10 tumors were treated with 1.4 MBq [²¹²Pb]VMT01. TILs were analyzed 7 days post treatment by flow cytometry using CD45 for leukocytes, CD3 for T cells, CD19 for B cells, CD4 for helper T cells, CD8 for cytotoxic T cells (FIG. 28A). Treatment with 1.4 MBq [²¹²Pb]VMT01 significantly enhanced the infiltration of CD45⁺ leukocytes and CD3⁺ T cells compared with control animals (FIG. 28B). Among CD45⁺ leukocytes, CD3⁺ T cells was increased to 39% by [²¹²Pb]VMT01 compared with 26% in control animals (FIG. 28B). Specifically, within the T cell population, [²¹²Pb]VMT01 induced greater tumor infiltrating CD4⁺ helper T cells (63%) and CD8⁺ cytotoxic T cells (29%) compared with control animals (FIG. 28B). These data demonstrate an immunomodulating effect of [²¹²Pb]VMT01 within the melanoma tumor microenvironment

### Discussion

In this study, a cooperative anti-tumor effect was demonstrated arising from the combination of ICIs and systemic targeted α-particle radiotherapy using ²¹²Pb-labeled MC1R-targeted peptide[²¹²Pb]VMT01. It was previously reported that several cyclic α-MSH analogs that were cyclized via Cu-catalyzed “click” chemistry. With the conjugation of bi-functional chelators, VMT01 were radiolabeled with bivalent radiometals (^{203/212}Pb²⁺), allowing for the employment of [²⁰³Pb]VMT01 as surrogate to determine the injected radioactivity of therapeutic [²¹²Pb]VMT01. The injected radioactivity was determined using 11 Gy in kidney dose as a maximum threshold, based on a previous study of α-TRT using a receptor targeted peptide ([²¹³Bi]DOTATATE) in which 11 Gy in kidney was identified as the LD5 in athymic nude mice. Of note, the injected radioactivity calculated from 11 Gy in kidney was not the maximal tolerated dose, considering that others reported injection of up to 7.4 MBq ²¹²Pb radiolabeled peptide in C57BL6 mice without observation of significant toxicities. In this study, acute toxicity was judged by the change in body weight. No significant toxicity was observed in any treatment cohort including the combination of [²¹²Pb]VMT01 and ICIs. [²¹²Pb]VMT01 treatment showed superior efficiency in both tumor-killing and immunogenicity (including 43% complete response rate in combination with ICIs) that relied on intact adaptive immunity. In the genetic modified Rag1 KO mice, the immunogenicity of [²¹²Pb]VMT01 was completed absent as a result of depleted adaptive immunity. Along with the in vivo evidence, FACS assays focusing on effector T cells demonstrated enhanced TILs, especially CD⁸⁺ T cells and CD⁴⁺ T cells in [²¹²Pb]VMT01-treated melanoma tumors, indicating strong immunogenic effect of [²¹²Pb]VMT01 α-TRT. Meanwhile, Morris et al. demonstrated that the presence of untreated distal tumors jeopardized the synergy between EBRT and immunotherapy in primary irradiated tumors via T_{reg} cell-mediated immunosuppression, emphasizing the importance of delivering radiation dose, even partial dose, to all sites and therefore create as many “hot tumor” sites as possible. Further studies are needed demonstrate the efficacy of combination of [²¹²Pb]VMT01 and ICIs in preclinical models with multiple tumor sites that displays heterogeneous expression of MC1R.

The immunogenicity of [²⁰³Pb]VMT01 could be attributable to its unique high linear energy transfer (LET, keV/micron) and resulted relative biological effectiveness (RBE). The interaction of α-particles in tissues leads to induction of condensed ionization along single relative short mean free path (maximum 100 micron). As a result, not only do α-particle interactions result in elevated levels of cellular damage (resulting in an increase in tumor associated neoantigens), but also result in a higher probability induced DNA double strand breaks (DSB) at low total absorbed doses. On the other hand, low-LET radiation (i.e., β-particles) require higher absorbed doses to achieve similar levels of DNA DSB. Meanwhile, studies have reported that α-particles induced more apoptotic cell death immediately after irradiation, compared with low LET radiation, which also contributes to enhanced neoantigen presentation.

Further suggesting the importance of dosing regimen, it was observed that suboptimal therapeutic outcomes were achieved when ICIs were combined with fractionated administration of [²¹²Pb]VMT01. Interestingly, the efficacy of [²¹²Pb]VMT01 monotherapy was not affected by fractionation, indicating that the tumor-killing effectiveness of α-TRT does not rely on fractionation as is observed for EBRT. However, only the single injection of [²¹²Pb]VMT01 induced potent anti-tumor cooperation with ICIs that led to significant complete tumor regression. Several factors might be considered in these observations. First, studies have demonstrated that ideal tumor response to ICIs is achieved when tumor burden is smallest. Thus, it may be that in an aggressive immunotolerant melanoma model, such as the B16-F10 melanoma tumor, the initial tumor dose imparted by [²¹²Pb]VMT01 must be sufficient to suppress the expansion of tumor size in order to allow for the activation of anti-tumor immunity. In this context, partial doses in each fraction might have led to inadequate control of the fast-growing tumor, which eventually overwhelmed the effectiveness of ICIs. Second, pre-existing TILs are important biomarkers for response to ICIs. In this study, significantly enhanced TILs were observed in the B16-F10 tumors seven days post [²¹²Pb]VMT01. Others observed significant influx of TILs in B16 tumors on day 14 post irradiation, whereas another group found infiltrated CD⁸⁺ T cells in B78 tumor on 12 days post irradiation. Thus, the compromised immunogenicity of fractionated [²¹²Pb]VMT01 might be attributable to the suboptimal influx of TILs upon the introduction of ICIs. Furthermore, it was observed that the TILs present in tumor microenvironment were also prone to be depleted by [²¹²Pb]VMT01 delivered in later fractions. Third, off-target expression of MC1R expression has been reported in monocytes, macrophages, lymphocytes, and neutrophils. Thus, it is also possible that later fractions [²¹²Pb]VMT01 delivered B16-F10 tumors imparted radiation dose to these intra-tumoral immune cells and thereby dampened the immunogenic effect.

Immunogenic cell death is defined as a specific type of apoptotic cell death that triggers adaptive immune immunity. Typically, immunogenic cell death is associated with expression of surface calreticulin, release of HMGB1, release of ATP, whereas vaccination and tumor re-challenge assays have been considered as a standard in vivo approach to validating immunogenic cell death inducers. In this study, the immunogenicity of [²¹²Pb]VMT01 was further determined by vaccination and tumor re-challenge assays, in which melanoma cells were killed by [²¹²Pb]VMT01 in cell culture flasks and then injected subcutaneously as cell-based vaccine in C57BL6 mice seven days before re-challenge with naïve melanoma cells. Slower growth of re-challenging tumors was observed in vaccinated animals compared with control cohorts. However, no complete tumor rejection was observed. This might be attributable to insufficient efficacy in one injection of cell-based vaccine, whereas stronger anti-tumor immune reaction might require multiple doses of vaccines (i.e., 2-3 doses) using more [²¹²Pb]VMT01 treated cells. Along with the vaccination assays, [²¹²Pb]VMT01 was capable of sensitizing immunotolerant melanoma cells to ICI treatments. The exposure to [²′2Pb]VMT01 in vitro led to generation of ICI-sensitive YUMM-PR and B16-PR cells. Generally, radiotherapy has been recognized as a potent inducer of immunogenic cell death that synergizes the efficacy of ICIs. A number of mechanistic pathways are known to be involved in the enhanced anti-tumor immune response that is induced by ionizing radiation. These include induction of the release of DNA and RNA into cytoplasm; induced Type I IFN responses; promotion of the release of danger signals such as damage-associated molecular patterns (DAMPs); activation of the STING signaling pathway; induction of increased expression of major histocompatibility complex class I (MHC I) proteins on the cancer cell surface; and enhanced presentation of tumor-associated antigens to immune systems via antigen presenting cells. More important, the delivery of radiation doses to multiple tumor sites has been considered beneficial to overcome tumor heterogeneity and immunotolerance by creating more “hot” tumor sites. Along these lines, targeted radionuclide therapy (TRT) such as [²¹²Pb]VMT01 is emerging as an effective approach to systemically deliver α-particle radiation that not only efficiently eliminates micrometastasis, but also induces anti-tumor immunity to enhance the efficacy of immunotherapies in a cooperative, potentially synergistic manner.

### CONCLUSIONS

In this study, ²¹²Pb radiolabeled peptide [²¹²Pb]VMT01 targeting MC1R was used to deliver α-particle radiation to melanoma cells. Robust anti-tumor cooperation between [²¹²Pb]VMT01 and systemic ICIs immunotherapy was observed in preclinical melanoma models. This cooperation relies on the intact adaptive immunity and immunogenicity of [²¹²Pb]VMT01. In addition, we have demonstrated that [²¹²Pb]VMT01 induces immunogenic cell death, tumor infiltrating lymphocytes, and sensitizes immunotolerant melanoma tumor to ICIs treatments.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A chelating agent that stably complexes a radionuclide, said radionuclide being suitable for therapeutic treatment and/or imaging of a cancerous malignancy, whereby the radiolabeled drug used for imaging and/or therapy of cancer;
wherein said chelating agent stably complexes a radionuclide, said radionuclide being suitable for therapeutic treatment and/or imaging of a cancerous malignancy.

2. The chelating agent of claim 1 that is selected from the group of compounds consisting essentially of:
S-2-(4-Nitrobenzyl) 1,4,7,10-tetraazacyclododecane;
1,4,7,10-Tetraazacyclododecane-1,4,7-tri(carbamoylmethyl)-10-acetic acid;
S-2-(4-Nitrobenzyl)-1,4,7,10-tetraazacyclododecane tetraacetic acid;
S-2-(4-Aminobenzyl)-1,4,7,10-tetraazacyclododecane tetraacetic acid;
S-2-(4-Aminobenzyl)-1,4,7,10-tetraazacyclododecane tetra-tert-butylacetate;
S-2-(4-Isothiocyanatobenzyl)-1,4,7,10-tetraazacyclododecane tetraacetic acid;
1,4,7,10-Tetraazacyclododecane-1,4,7-tris-tert-butyl acetate-10-acetic acid;
1,4,7,10-Tetraazacyclododecane-1,4,7-tris-tert-butyl acetate-10-succinimidyl acetate;
1,4,7,10-Tetraazacyclododecane-1,4,7-tris-tert-butyl acetate-10-maleimidoethylacetamide;
1,4,7,10-Tetraazacyclododecane-1,4,7-tris-acetic acid-10-maleimidoethylacetamide;
1,4,7,10-Tetraazacyclododecane-1,4,7-tris-tert-butyl acetate-10-(N-a-Fmoc-N-e-acetamido-L-lysine);
1,4,7,10-Tetraazacyclododecane-1,4,7-tris(t-butyl acetate)-10-(3-butynylacetamide);
1,4,7,10-Tetraazacyclododecane-1,4,7-tris(t-butyl-acetate)-10-(amino ethylacetamide);
1,4,7,10-Tetraazacyclododecane-1,4,7-tris-tert-butyl acetate-10-(azidopropyl ethylacetamide);
1,4,7,10-Tetraazacyclododecane-1,4,7-tris(t-butyl acetate)-10-(4-aminobutyl)acetamide;
1,4,7,10-Tetraazacyclododecane-1,4,7,10-tetraacetic acid mono-N-hydroxysuccinimide ester;
1,4,7,10-Tetraazacyclododecane-1,4,7-tris(acetic acid)-10-(2-thioethyl)acetamide or other variation of DOTA;
S-2-(4-Aminobenzyl)-diethylenetriamine pentaacetic acid or other variation of DTPA;
3,6,9,15-Tetraazabicyclo[9.3.1] pentadeca-1(15),11,13-triene-4-S-(4-aminobenzyl)-3,6,9-triacetic acid or other variation on pentadeca macrocycle;
1-Oxa-4,7,10-tetraazacyclododecane-5-S-(4-aminobenzyl)-4,7,10-triacetic acid or other variation on oxo-substituted macrocycle;
2-S-(4-Isothiocyanatobenzyl)-1,4,7-triazacyclononane-1,4,7-triacetic acid or other variation on cyclononane; and
1-(4-isothiocyanatophenyl)-3-[6,17-dihydroxy-7,10,18,21-tetraoxo-27-(N-acetylhydroxylamino)-6,11,17, 22-tetraazaheptaeicosine] thiourea or other variation on deferoxamine.

3. The chelating agent of claim 1 that is radiometallated or radiolabeled with a radionuclide, said radionuclide being suitable for therapeutic treatment and/or imaging of a cancerous malignancy.

4. The chelating agent of claim 3 that is radiometallated or radiolabeled with a radionuclide that is suitable for therapeutic treatment and/or imaging of MCR1 expression cancerous malignancy.

5. The chelating agent of claim 4 that is radiometallated or radiolabeled with a radionuclide that is selected from the group consisting of Ga-68, In-111, Pb-203, Pb-212, F-18, C-11, Zr-89, Sc-44, Tc-99m, Y-90, Pb-212, Bi-212, Bi-213, At-211, Lu-177, Ac-225, Cu-64, Cu-67, and Re-188.

6. The chelating agent of claim 1 that is DOTA.

7. The chelating agent of claim 1 that is a Pb-specific chelator (PSC).

8. The chelating agent of claim 1 that has the following structure:

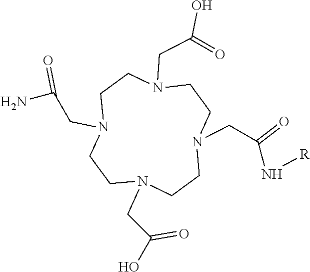

9. The chelating agent of claim 8 that is radiolabeled with a radionuclide selected from the group consisting of Pb-203, Pb-212, and Bi-212.

10. A chelating agent having the following structure:

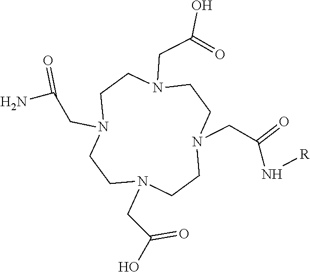

11. A method of treating cancer in a patient in need thereof, comprising administering to the patient a chelating agent that is used to bind the radionuclide for diagnostic imaging or therapy for cancer.

12. The method of claim 11 whereby the chelating agent is radiometallated or radiolabeled with a radionuclide, said radionuclides being suitable for therapeutic treatment and/or imaging of a cancerous malignancy.

13. The method of claim 11 whereby the chelating agent is based on a compound selected from the group consisting essentially of:
S-2-(4-Nitrobenzyl) 1,4,7,10-tetraazacyclododecane;
1,4,7,10-Tetraazacyclododecane-1,4,7-tri(carbamoylmethyl)-10-acetic acid;
S-2-(4-Nitrobenzyl)-1,4,7,10-tetraazacyclododecane tetraacetic acid;
S-2-(4-Aminobenzyl)-1,4,7,10-tetraazacyclododecane tetraacetic acid;
S-2-(4-Aminobenzyl)-1,4,7,10-tetraazacyclododecane tetra-tert-butylacetate;
S-2-(4-Isothiocyanatobenzyl)-1,4,7,10-tetraazacyclododecane tetraacetic acid;
1,4,7,10-Tetraazacyclododecane-1,4,7-tris-tert-butyl acetate-10-acetic acid;
1,4,7,10-Tetraazacyclododecane-1,4,7-tris-tert-butyl acetate-10-succinimidyl acetate;
1,4,7,10-Tetraazacyclododecane-1,4,7-tris-tert-butyl acetate-10-maleimidoethylacetamide;
1,4,7,10-Tetraazacyclododecane-1,4,7-tris-acetic acid-10-maleimidoethylacetamide;
1,4,7,10-Tetraazacyclododecane-1,4,7-tris-tert-butyl acetate-10-(N-a-Fmoc-N-e-acetamido-L-lysine);
1,4,7,10-Tetraazacyclododecane-1,4,7-tris(t-butyl acetate)-10-(3-butynylacetamide);
1,4,7,10-Tetraazacyclododecane-1,4,7-tris(t-butyl-acetate)-10-(amino ethylacetamide);
1,4,7,10-Tetraazacyclododecane-1,4,7-tris-tert-butyl acetate-10-(azidopropyl ethylacetamide);
1,4,7,10-Tetraazacyclododecane-1,4,7-tris(t-butyl acetate)-10-(4-aminobutyl)acetamide;
1,4,7,10-Tetraazacyclododecane-1,4,7,10-tetraacetic acid mono-N-hydroxysuccinimide ester;
1,4,7,10-Tetraazacyclododecane-1,4,7-tris(acetic acid)-10-(2-thioethyl)acetamide or other variation of DOTA;
S-2-(4-Aminobenzyl)-diethylenetriamine pentaacetic acid or other variation of DTPA;
3,6,9,15-Tetraazabicyclo[9.3.1] pentadeca-1(15),11,13-triene-4-S-(4-aminobenzyl)-3,6,9-triacetic acid or other variation on pentadeca macrocycle;
1-Oxa-4,7,10-tetraazacyclododecane-5-S-(4-aminobenzyl)-4,7,10-triacetic acid or other variation on oxo-substituted macrocycle;
2-S-(4-Isothiocyanatobenzyl)-1,4,7-triazacyclononane-1,4,7-triacetic acid or other variation on cyclononane; and
1-(4-isothiocyanatophenyl)-3-[6,17-dihydroxy-7,10,18,21-tetraoxo-27-(N-acetylhydroxylamino)-6,11,17, 22-tetraazaheptaeicosine] thiourea or other variation on deferoxamine.

14. The method of claim 11, wherein the cancer is melanoma.

15. The method of claim 11, wherein the chelating agent is administered orally or parenterally.

16. The method of claim 11, wherein the chelating agent is a Pb-specific chelator (PSC).

17. The method of claim 11, whereby the chelating agent has the following structure:

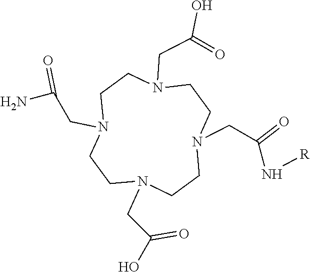

18. The method of claim 11 whereby the chelating agent is radiolabeled with a radionuclide that is selected from the group consisting of Pb-203, Pb-212, and Bi-212.

19. The method of claim 11, wherein the chelating agent is part of a conjugate having the structural formula:

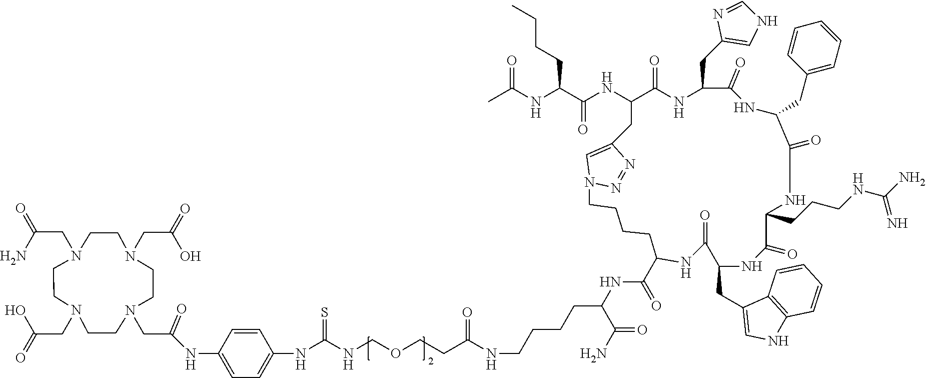

20. The method of claim 14, wherein the conjugate administered for a time period of at least 7 days.
